SEQUENCE LISTING (20 sequences)

SEQ ID NO: 1 - protein, 9 aa, Artificial Sequence
  Chemerin-9
YFPGQFAFS

SEQ ID NO: 2 - protein, 9 aa, Artificial Sequence
  peptide derived from chemerin-9
  MOD_RES (1)..(1): Tam (= 6-carboxytetramethylrhodamine) modification @ aa position 1
YFPGQFAFS

SEQ ID NO: 3 - protein, 9 aa, Artificial Sequence
  peptide derived from chemerin-9
  MOD_RES (1)..(1): Tam-EG(4) modification @ aa position 1
YFPGQFAFS

SEQ ID NO: 4 - protein, 9 aa, Artificial Sequence
  peptide derived from chemerin-9
  DISULFID (4)..(9)
  MOD_RES (4)..(4): D-cysteine @ aa position 4
YFPCQFAFC

SEQ ID NO: 5 - protein, 9 aa, Artificial Sequence
  peptide derived from chemerin-9
  MOD_RES (1)..(1): Tam (= 6-carboxytetramethylrhodamine) modification @ aa position 1
  DISULFID (4)..(9)
  MOD_RES (4)..(4): D-cysteine @ aa position 4
YFPCQFAFC

SEQ ID NO: 6 - protein, 9 aa, Artificial Sequence
  peptide derived from chemerin-9
  DISULFID (4)..(7)
  MOD_RES (4)..(4): D-cysteine @ aa position 4
YFPCQFCFS

SEQ ID NO: 7 - protein, 9 aa, Artificial Sequence
  peptide derived from chemerin-9
  SITE (4)..(9): Amino acid side chains @ aa positions 4 and 9 connected via CH2 group
  MOD_RES (4)..(4): D-cysteine @ aa position 4
YFPCQFAFC

SEQ ID NO: 8 - protein, 9 aa, Artificial Sequence
  peptide derived from chemerin-9
  MOD_RES (1)..(1): Tam (= 6-carboxytetramethylrhodamine) modification @ aa position 1
  SITE (4)..(9): Amino acid side chains @ aa positions 4 and 9 connected via CH2 group
  MOD_RES (4)..(4): D-cysteine @ aa position 4
YFPCQFAFC

SEQ ID NO: 9 - protein, 9 aa, Artificial Sequence
  peptide derived from chemerin-9
  MOD_RES (4)..(4): D-cysteine @ aa position 4
  SITE (4)..(9): Amino acids side chains at positions 4 and 9 connected via CH2 group
  MOD_RES (9)..(9): Xaa @ aa position 9 is D-homocysteine
YFPCQFAFX

SEQ ID NO: 10 - protein, 9 aa, Artificial Sequence
  peptide derived from chemerin-9
  MOD_RES (1)..(1): Tam (= 6-carboxytetramethylrhodamine) modification @ aa position 1
  MOD_RES (4)..(4): D-cysteine @ aa position 4
  SITE (4)..(9): Amino acid side chains at positions 4 and 9 connected via CH2 group
  MOD_RES (9)..(9): Xaa @ aa position 9 is D-homocysteine
YFPCQFAFX

SEQ ID NO: 11 - protein, 9 aa, Artificial Sequence
  peptide derived from chemerin-9
  MOD_RES (1)..(1): D-tyrosine @ aa position 1
  MOD_RES (4)..(4): D-cysteine @ aa position 4
  SITE (4)..(9): Amino acid side chains at positions 4 and 9 connected via CH2 group
  MOD_RES (9)..(9): Xaa @ aa position 9 is D-homocysteine
YFPCQFAFX

SEQ ID NO: 12 - protein, 9 aa, Artificial Sequence
  peptide derived from chemerin-9
  MOD_RES (1)..(1): D-tyrosine @ aa position 1
  MOD_RES (4)..(4): D-cysteine @ aa position 4
  DISULFID (4)..(9)
  MOD_RES (7)..(7): Lys side chain @ aa position 7 modified with Tam
YFPCQFKFC

SEQ ID NO: 13 - protein, 9 aa, Artificial Sequence
  peptide derived from chemerin-9
  MOD_RES (4)..(4): Xaa @ aa position 4 is D-homocysteine
  DISULFID (4)..(9)
YFPXQFAFC

SEQ ID NO: 14 - protein, 9 aa, Artificial Sequence
  peptide derived from chemerin-9
  MOD_RES (4)..(4): Xaa @ aa position 4 is D-homocysteine
  DISULFID (4)..(9)
YFPXQFAWC

SEQ ID NO: 15 - protein, 9 aa, Artificial Sequence
  peptide derived from chemerin-9
  MOD_RES (1)..(1): D-tyrosine @ aa position 1
  MOD_RES (4)..(4): Xaa @ aa position 4 is D-homocysteine
  DISULFID (4)..(9)
YFPXQFAWC

SEQ ID NO: 16 - protein, 9 aa, Artificial Sequence
  peptide derived from chemerin-9
  MOD_RES (1)..(1): EG(4) modification @ aa position 1
  MOD_RES (4)..(4): Xaa @ aa position 4 is D-homocysteine
  DISULFID (4)..(9)
YFPXQFAFC

SEQ ID NO: 17 - protein, 9 aa, Artificial Sequence
  peptide derived from chemerin-9
  MOD_RES (1)..(1): Tam-EG(4) modification @ aa position 1
  MOD_RES (4)..(4): Xaa @ aa position 4 is D-homocysteine
  DISULFID (4)..(9)
YFPXQFAFC

SEQ ID NO: 18 - protein, 9 aa, Artificial Sequence
  peptide derived from chemerin-9
  MOD_RES (1)..(1): EG(4) modification @ aa position 1
  MOD_RES (4)..(4): Xaa @ aa position 4 is D-homocysteine
  DISULFID (4)..(9)
YFPXQFAWC

SEQ ID NO: 19 - protein, 9 aa, Artificial Sequence
  peptide derived from chemerin-9
  MOD_RES (1)..(1): Tam-EG(4) modification @ aa position 1
  MOD_RES (4)..(4): Xaa @ aa position 4 is D-homocysteine
  DISULFID (4)..(9)
YFPXQFAWC

SEQ ID NO: 20 - protein, 13 aa, Artificial Sequence
  peptide derived from chemerin-9
  MOD_RES (1)..(1): ACETYLATION
  MOD_RES (8)..(8): Xaa @ aa position 8 is D-homocysteine
  DISULFID (8)..(13)
GFLGYFPXQFAFC

(19) United States
(12) Patent Application Publication
(10) Pub. No.: US 2023/0303647 A1
(43) Pub. Date: Sep. 28, 2023

(54) CYCLIC CHEMERIN-9 DERIVATIVES

(71) Applicant: Bayer Aktiengesellschaft, Leverkusen (DE)

(72) Inventors: Jan Robert KRÄHLING, Düsseldorf (DE); Bernd RIEDL, Wuppertal (DE); Annette BECK-SICKINGER, Leipzig (DE); Tobias FISCHER, Leipzig (DE); Anne CZERNIAK, Leipzig (DE); Sylvia ELS-HEINDL, Leipzig (DE)

(73) Assignee: Bayer Aktiengesellschaft, Leverkusen (DE)

(21) Appl. No.: 18/020,878

(22) PCT Filed: Aug. 10, 2021
(86) PCT No.: PCT/EP2021/072236

§ 371 Date: Feb. 10, 2023

(30) Foreign Application Priority Data
Aug. 12, 2020 (EP) 20190794.6

(51) Int. Cl. C07K 14/52

(52) U.S. Cl. CPC C07K 14/523; A61K 38/00

(57) ABSTRACT

The present invention relates to cyclic chemerin-9 derivatives of general formula (I) as described and defined herein, methods of preparing said peptides, and the use of said compounds for the treatment or prophylaxis of diseases, in particular cancer, diabetes, obesity and inflammatory disorders.

The present invention relates to cyclic chemerin-9 derivatives of general formula (I) as described and defined herein, methods of preparing said peptides, and the use of said compounds for the treatment or prophylaxis of diseases, in particular cancer, diabetes, obesity and inflammatory disorders.

### BACKGROUND

Chemerin is a small adipokine that was first identified in 2003 by Wittamer et al. (Wittamer, Franssen et al., Specific recruitment of antigen-presenting cells by chemerin, a novel processed ligand from human inflammatory fluids. J Exp Med, 2003, 198(7): 977-985). It is mainly expressed by skin, liver, and adipose tissue (Roh, Song et al., Chemerin—a new adipokine that modulates adipogenesis via its own receptor. Biochem Biophys Res Commun. 2007, 362(4): 1013-1018, Banas, Zabieglo et al., Chemerin is an antimicrobial agent in human epidermis. PLoS One, 2013, 8(3): e58709). Chemerin is expressed in its inactive form, the 163 amino acid preprochemerin, which is secreted after N-terminal truncation of a signaling peptide. The resulting, inactive prochemerin can be activated through C-terminal processing by various proteases, e.g. kallikrein-7 (Schultz, Saalbach et al., Proteolytic activation of prochemerin by kallikrein 7 breaks an ionic linkage and results in C-terminal rearrangement. Biochem J, 2013, 452(2): 271-280), cathepsin G (Zabel, Allen et al., Chemerin activation by serine proteases of the coagulation, fibrinolytic, and inflammatory cascades. J Biol Chem, 2005, 280(41): 34661-34666) or plasmin (Yamaguchi, Du et al., Proteolytic cleavage of chemerin protein is necessary for activation to the active form. Chem157S, which functions as a signaling molecule in glioblastoma. J Biol Chem, 2011, 286(45): 39510-39519) to give active chemerin. The most active isoform is formed by cleavage after serine 157 (numbering for the human protein) and is consequently referred to as ChemS157. The C-terminal part of this protein is essential for biological activity, and a peptide consisting of the ultimate nine amino acids shows an activity comparable to the full-length protein (Wittamer, Gregoire et al., The C-terminal nonapeptide of mature chemerin activates the chemerin receptor with low nanomolar potency. J Biol Chem, 2004, 279(11): 9956-9962). This peptide is widely referred to as chemerin-9.

Chemerin binds to the three receptors chemokine-like receptor 1 (CMKLR1), G protein-coupled receptor 1 (GPR1) and chemokine (CC-motif) receptor-like 2 (CCRL2). (Wittamer, Franssen et al., Specific recruitment of antigen-presenting cells by chemerin, a novel processed ligand from human inflammatory fluids. J Exp Med, 2003, 198(7): 977-985, Bamea, Strapps et al., The genetic design of signaling cascades to record receptor activation. Proc Natl Acad Sci USA, 2008, 105(1): 64-69. Zabel. Nakae et al., Mast cell-expressed orphan receptor CCRL2 binds chemerin and is required for optimal induction of IgE-mediated passive cutaneous anaphylaxis. The Journal of experimental medicine, 2008, 205(10): 2207-2220) GPR1 and CMKLR1 are closely related, but only the latter induces G protein signaling. (De Henau, Degroot et al., Signaling Properties of Chemerin Receptors CMKLR1, GPR1 and CCRL2. PLoS One, 2016, 11(10): e0164179) GPR1 is often described as a mere decoy receptor although it induces down-stream signaling through the RhoA/ROCK pathway. (Rourke. Dranse et al., CMKLR1 and GPR1 mediate chemerin signaling through the RhoA ROCK pathway. Mol Cell Endocrinol, 2015, 417(36-51) In contrast, the atypical chemokine receptor CCRL2 fails to trigger intracellular signaling events or internalization and is thought to act by increasing local chemerin concentrations. (Zabel, Nakae et al., Mast cell-expressed orphan receptor CCRL2 binds chemerin and is required for optimal induction of IgE-mediated passive cutaneous anaphylaxis. The Journal of experimental medicine, 2008, 205(10): 2207-2220) The CMKLR1 is expressed by adipocytes, but also by tissue specific macrophages and dendritic cells. (Wittamer, Franssen et al., Specific recruitment of antigen-presenting cells by chemerin, a novel processed ligand from human inflammatory fluids. J Exp Med, 2003, 198(7): 977-985, Luangsay, Wittamer et al., Mouse ChemR23 is expressed in dendritic cell subsets and macrophages, and mediates an anti-inflammatory activity of chemerin in a lung disease model. J Immunol, 2009, 183(10): 6489-6499) Activation of the CMKLR1 by chemerin results in the recruitment of these cells to sites of inflammation, and treatment of chondrocytes and synoviocytes with chemerin triggers the release of pro-inflammatory cytokines such as TNF-α, CCL2 and interleukins. (Berg, Sveinbjõrnsson et al., Human articular chondrocytes express ChemR23 and chemerin; ChemR23 promotes inflammatory signalling upon binding the ligand chemerin 21-157. Arthritis Res Ther, 2010, 12(6): R228, Kaneko, Miyabe et al., Chemerin activates fibroblast-like synoviocytes in patients with rheumatoid arthritis. Arthritis Res Ther, 2011, 13(5): R158) In contrast, a C-terminal derivate of chemerin, referred to as C15, is described to have potent anti-inflammatory effects in a murine model of peritonitis. (Cash, Hart et al., Synthetic chemerin-derived peptides suppress inflammation through ChemR23. J Exp Med, 2008, 205(4): 767-775) This peptide also seems to improve wound healing in vivo, as was shown by Cash et al in a mouse model. (Cash, Bass et al., Resolution mediator chemerin15 reprograms the wound microenvironment to promote repair and reduce scarring. Curr Biol. 2014, 24(12): 1406-1414)

Serum levels of chemerin are correlated with the body mass index (Bozaoglu, Bolton et al., Chemerin is a novel adipokine associated with obesity and metabolic syndrome. Endocrinology, 2007, 148(10): 4687-4694) and it is therefore not surprising that chemerin has gained increasing interest with respect to its role in obesity-related diseases. Treatment of 3T3-L1 cells with chemerin increased insulin signaling and insulin-induced glucose uptake. (Takahashi, Takahashi et al., Chemerin enhances insulin signaling and potentiates insulin-stimulated glucose uptake in 3T3-L1 adipocytes. FEBS Lett, 2008, 582(5): 573-578). This promising property for the treatment of diabetes seems to be retained in chemerin-9: In a mouse model of pancreatic diabetes mellitus, treatment with chemerin-9 showed a significant alleviation of glucose intolerance by elevating the expression levels of the glucose transporter glut2 and the insulin promoter factor 1. (Tu, Yang et al., Regulatory effect of chemerin and therapeutic efficacy of chemerin 9 in pancreatogenic diabetes mellitus. Mol Med Rep, 2020, 21(3): 981-988) Apart from the roles in inflammation and obesity, there is emerging evidence that chemerin is also a potential target for the treatment of cancer. Chemerin promotes the invasion of squamous oesophageal cancer cells (Kumar, Kandola et al., The role of chemerin and ChemR23 in stimulating the invasion of squamous oesophageal cancer cells. Brit J Cancer, 2016, 114(10): 1152-1159), and inhibition of the chemerin/CMKLR1 axis in neuroblastoma cells reduces tumor growth and cell viability in vivo. (Tummler, Snapkov et al., Inhibition of chemerin CMKLR1 axis in neuroblastoma cells reduces clonogenicity and cell viability in vitro and impairs tumor growth in vivo. Oncotarget, 2017, 8(56): 95135-95151) In colorectal cancer, the CMKLR1 is proposed to be important for tumor growth by promoting angiogenesis. (Kiczmer, Senkowska et al., Assessment of CMKLR1 level in colorectal cancer and its correlation with angiogenic markers. Exp Mol Pathol, 2020, 113(104377) On the other hand, chemerin suppressed metastases of hepatocellular carcinoma in mice. (Li, Yin et al., Chemerin suppresses hepatocellular carcinoma metastasis through CMKLR1-PTEN-Akt axis. British Journal of Cancer, 2018, 118(10): 1337-1348).

These results clearly demonstrate the potential of chemerin-derived peptides for the treatment of different diseases, but the native peptides suffer from extremely low plasma stability, calling for more stable and potent derivates. (Bandholtz. Wichard et al., Molecular evolution of a peptide GPCR ligand driven by artificial neural networks. PLoS One, 2012, 7(5): e36948) Previous studies aiming to develop more stable chemerin-9 derivates focused solely on introducing unnatural amino acids, which led to the development of derivates with a plasma half-life of four hours. (Shimamura, Matsuda et al., Identification of a stable chemerin analog with potent activity toward ChemR23. Peptides, 2009, 30(8): 1529-1538) This still is far from optimal for a potential therapeutic use. Cyclic chemerin-9 derivatives have been described in Journal of Medicinal Chemistry 2021 64 (6), 3048-3058.

The present invention generally relates to cyclic chemerin-9 derivatives with improved plasma stability and methods of making and using the same.

The present invention provides compounds of the general formula (I)

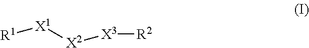

wherein
R¹ is absent
   or
   represents 6-Carboxytetramethylrhodamine (Tam), ##C(O)R³, C₈-C₂₀ fatty acid or the sequence R⁴GFLG##, R⁴—C═N—NH-##, R⁴—S—S-##, R⁴—N═N-##, R-Valin-Citrullin-##, R⁴—C(O)O-## or R⁴NH—C(O)O-##
      wherein
      ## marks the attachment to the terminal amino group of X¹,
      R³ represents C₁-C₆-alkylene, aryl, heteroaryl, C₃-C₈-cycloalkyl or C₃-C₇-heterocycloalkyl,
         wherein C₁-C₆-alkylene is up to trisubstituted identically or differently by a radical selected from the group consisting of hydroxyl, methoxy, ethoxy, carboxy, amino and halogen,
         wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
      R⁴ represents

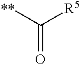

wherein
R⁵ represents C₁-C₆-alkylene, aryl, heteroaryl, C₃-C₈-cycloalkyl or C₃-C₇-heterocycloalkyl,
wherein C₁-C₆-alkylene is up to trisubstituted identically or differently by a radical selected from the group consisting of hydroxyl, methoxy, ethoxy, carboxy, amino and halogen,
wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
or
represents a group of the formula (IIIa)

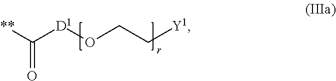

wherein
** marks the attachment to a nitrogen atom,
D¹ is C₁-C₄-alkylene,
Y¹ is selected from the group consisting of hydroxyl, methoxy, ethoxy, carboxy, carboxamide or amino
   wherein amino might be substituted with 6-carboxytetramethyirhodamine (Tam) via an amide bond,
and
r represents an integer of from 2 to 15,
R² represents a group of the formula (II)

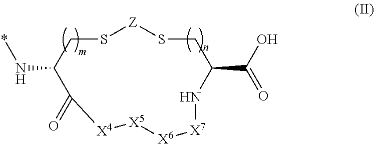

or
represents a group of the formula (III)

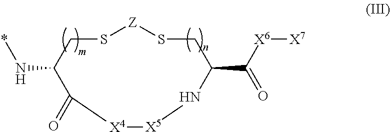

wherein
* represents the attachment to the carbonyl atom of the carboxy group of X³,
Z represents a bond or —CH₂—,
m represents 1 or 2,
n represents 1 or 2,
X¹ represents a natural amino acid selected from a list consisting of L, I, F, H, M, W, Y or y or an unnatural amino acid selected from a list consisting of L-Norleucine (Nle), 2,3,3a,4,5,6,7,7a-Octahydroindole-2-carboxylic acid (Oic), 4-Bromophenylalanine ((4-Bromo)F), 2,5-Difluorophenylalanine ((2,5-Difluoro)F), 2-Chlorophenylalanine ((2-Chlor)F), 3-Chlorophenylalanine ((3-Chlor)F), 4-Chlorophenylalanine ((4-Chloro)F), 2-Bromophenylalanine ((2-Bromo)F), 3-Bromophenylalanine ((3-Bromo)F), 4-Bromophenylalanine ((4-Bromo)F), 2-Fluorophenylalanine ((2-Fluoro)F), 3-Fluorophenylalanine ((3-Fluoro)F), 4-Fluorophenylalanine ((4-Fluoro)F), (2,5-difluoro-phenylalanine, 2-Methylphenylalanine ((2-Me)F), 3-Methyl-phenylalanine ((3-Me)F), 4-Methylphenylalanine ((4-Me)F), (2S)-3-(2,3-difluorophenyl)-2-aminopropanoic acid, Phenylglycine (Phg)N-Phenylglycine ((N-Ph)G), 3-Chlorophenylglycine ((3-Chloro-Ph)G), 3-(1,3-Benzothiazol-2-yl)-alanine 1-Benzyl-histidine (H(1-Bn)), 1-Methyl-histidine (H(1-Me)), 3-Methylhistidine (3-Me)H), 2-Pyridylalanine (2-Pal), 3-Pyridylalanine (3-Pal), 4-Pyridylalanine (4-Pal), 3-(Aminomethyl)benzoic acid, 1-Napthylalanine (1-Nal), 2-Napthylalanine (2-Nal), (2R)-Amino-(1-methyl-1H-indazol-5-yl)acetic acid and (2S)-3-(indol-4-yl)-2-(amino)propanoic acid, whereas any natural amino acid and/or unnatural amino acid from that list can be in D- or L-stereoconfiguration,
X² represents a natural amino acid selected from a list consisting of L, I, F, H, M, W or Y or an unnatural amino acid selected from a list consisting of L-Norleucine (Nle), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F). (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Napthylalanine (1-Nal), L-2-Napthylalanine (2-Nal), (2R)-Amino-(1-methyl-1H-indazol-5-yl)acetic acid and (2S)-3-(indol-4-yl)-2-(amino)propanoic acid,
X³ represents the natural amino acid P. or an unnatural amino acid selected from a list consisting of 2,3,3a,4,5,6,7,7a-Octahydroindole-2-carboxylic acid (Oic). L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline ((3S—OH)P, L-Pipecolic acid (Pip), (1R,3S,5R)-2-azabicyclo[3.1.0]hexane-3-carboxylic acid, (6S)-5-Azaspiro-[2.4]heptane-6-carboxylic acid, rel-(1R,3R,5R,6R)-6-(trifluoromethyl)-2-azabicyclo[3.1.0]hexane-3-carboxylic acid, (2S)-2-Amino-4,4,4-trifluorobutanoic acid, (2S,3aS,6aS)-octahydrocyclopenta[b]^{¬}pyrrole-2-carboxylic acid, trans-4-fluoroproline ((trans-4-Fluoro)P), (2S,4S)-4-fluoroproline ((cis-4-Fluoro)P), L-4,4-difluoroproline ((Difluoro)P), rel-(3R,6R)-1,1-difluoro-5-azaspiro[2.4]heptane-6-carboxylic acid (enantiomer 1) and rel-(3R,6R)-1,1-difluoro-5-azaspiro[2.4]heptane-6-carboxylic acid (enantiomer 2),
X⁴ represents any natural amino acid or an unnatural amino acid, whereas any natural amino acid and/or unnatural amino acid can be in D- or L-stereoconfiguration,
X⁵ represents a natural amino acid selected from a list consisting of F, H, W or Y or an unnatural amino acid selected from a list consisting of Cyclohexylalanine (Cha), 2,3,3a,4,5,6,7,7a-Octahydrindole-2-carboxylic acid (Oic), L-4-Bromophenylalanine ((4-Bromo)F), 2,5-Difluoro-L-phenylalanine ((2,5-Difluoro)F), 2-Chlor-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bomophenylalanine ((4-Bromo)F), 2-Fluoro-L-phenylalanine ((2-Fluor)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Napthylalanine (1-Nal), L-2-Napthylalanine (2-Nal), (2R)-Amino-(1-methyl-1H-indazol-5-yl)acetic acid and (2S)-3-(indol-4-yl)-2-(amino)propanoic acid,
X⁶ represents any natural amino acid or an unnatural amino acid, whereas any natural amino acid and/or unnatural amino acid can be in D- or L-stereoconfiguration,
   wherein any natural amino acid or an unnatural amino acid bearing an amino group might be substituted with 6-Carboxytetramethylrhodamine (Tam) or ##C(O)R³,
      wherein
      ## marks the attachment to the terminal amino group of X¹,
      R³ represents C₁-C₆-alkylene, aryl, heteroaryl, C₃-C₈-cycloalkyl or C₃-C₇-heterocycloalkyl,
      wherein C₁-C₆-alkylene is up to trisubstituted identically or differently by a radical selected from the group consisting of hydroxyl, methoxy, ethoxy, carboxy, amino and halogen,
      wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
X⁷ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Naphthylalanine (1-Nal). L-2-Naphthylalanine (2-Nal), (2R)-Amino-(1-methyl-1H-indazol-5-yl)acetic acid and (2S)-3-(indol-4-yl)-2-(amino)propanoic acid,
or a pharmaceutically acceptable salt, hydrate, solvate or solvate of the salt thereof,
   with the proviso, that compound YFP[cQFAFC] is excluded.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are cited below as working examples and the salts, solvates and solvates of the salts thereof if the compounds that are encompassed by formula (I) and are mentioned below are not already salts, solvates and solvates of the salts.

Compounds of the invention are likewise N-oxides and S-oxides of the compounds of the formula (I) and the salts, solvates and solvates of the salts thereof.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

Throughout this specification, the word “comprise” or variations thereof such as “comprises” or “comprising” will be understood to imply the inclusion of a stated integer (or components) or group of integers (or components), but not the exclusion of any other integer (or components) or group of integers (or components). The singular forms “a”, “an” and “the” include the plurals unless the context clearly dictates otherwise. The term “including” and “containing” is used to mean “including but not limited to”, which expressions can be used interchangeably. In particular, the expression “compound containing a peptide” means a compound which contains a defined peptide sequence and which can optionally contain further chemical groups or substituents covalently bound to the peptide, e.g. amino acids, fatty acids, chemical groups to enhance pharmacodynamic or pharmacokinetic properties of the peptide or any other chemical groups. It is also to be understood that the expression “compound containing a peptide” explicitly includes the defined peptide sequence without any further chemical groups or substituents covalently bound to that peptide.

As used herein, the following terms have the meanings ascribed to them unless specified otherwise. “Essentially consisting of” is understood as a peptide being at least 80%, at least 85%, at least 90%, at least 95%, at least 96%, at least 97%, at least 98%, or at least 99% identical to the peptide it is compared to.

The terms “protein”. “polypeptide” and “peptide” are used interchangeably to refer broadly to a sequence of two or more amino acids linked together, preferable by peptide (amide) bonds. Peptide (amide) bonds are formed when the carboxyl group of one amino acid reacts with the amino group of another. It should be further understood that the terms “protein”, “polypeptide” and “peptide” do not indicate a specific length of a polymer of amino acids, nor is it intended to imply or distinguish whether the polypeptide is produced using recombinant techniques, chemical or enzymatic synthesis, or is naturally occurring. It should be further understood, that a peptide can contain one or more parts which are no amino acids under the definition of the present application. These parts are preferably present at the N- and C-terminal ends of the peptide.

The term “amino acid” or “any amino acid” as used herein refers to organic compounds containing amine (—NH₂) and carboxyl (—COOH) functional groups, along with a side chain and refers to any and all amino acids, including naturally occurring amino acids (e.g., α-L-amino acids), unnatural amino acids, modified amino acids, and non-natural amino acids. “Natural amino acids” include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The 20 proteinogenic, natural amino acids in the standard genetic code are listed in Table 2. The “non-standard” natural amino acids are pyrrolysine (found in methanogenic organisms and other eukaryotes), selenocysteine (present in many non-eukaryotes as well as most eukaryotes), and N-formylmethionine (encoded by the start codon AUG in bacteria, mitochondria and chloroplasts).

“Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, and N-methyl amino acids. Unnatural or non-natural amino acids also include modified amino acids. “Modified” amino acids include amino acids (e.g., natural amino acids) that have been chemically modified to include a group, groups, or chemical moiety not naturally present in the amino acid. According to the present invention preferred unnatural amino acids are listed in Table 1. Table 1 displays unnatural amino acids as D- and/or L-stereoisomers, however preferred unnatural amino acids according to the invention are both D- and L-stereoisomers of unnatural amino acids listed in Table 1.

Table 1: Preferred unnatural amino acids
(1R,2R)-2-Amino-1-cyclopentanecarboxylic acid (R,R-ACPC)
(1R,3S)-3-(Amino)cyclopentanecarboxylic acid
(1R,3S,5R)-2-azabicyclo[3.1.0]hexane-3-carboxylic acid
(1S,2S)-2-Amino-1-cyclopentanecarboxylic acid (S,S-ACPC)
(1S,2S,5R)-3-azabicyclo[3.1.0]hexane-2-carboxylic acid
(1R,2S,5S)-3-Azabicyclo[3.1.0]hexane-2-carboxylic acid
(1S,3R)-3-(Amino)cyclopentanecarboxylic acid
(1S,3R)-3-(Amino)cyclopentanecarboxylic acid
(1S,3R,4R)-2-Azabicyclo[2.2.1]heptane-3-carboxylic acid
(2S)-2-(Amino)-2-[(1S,3R)-3-hydroxycyclohexyl]acetic acid
(2S)-2-(Amino)-2-[(1S,3S)-3-hydroxycyclohexyl]acetic acid
(2R)-Amino-(1-methyl-1H-indazol-5-yl)acetic acid
(2S)-2-Amino-5-methyl-hexanoic acid
(2S)-2-[(3R)-3-Amino-2-oxopyrrolidin-1-yl]-4-methylpentanoic acid
(2S)-2[(amino)-2-(tetrahydro-2H-pyran-4-yl)]acetic acid
(2S)-2-amino-3-(1-methylcyclopropyl)propanoic acid
(2S)-2-amino-3-(2,3,4,5,6-pentafluorophenyl)propanoic acid
(2S)-2-amino-3-(4-tert-butylphenyl)propanoic acid
(2S)-2-Amino-4-(benzylamino)-4-oxobutanecarboxylic acid
(2S)-2-amino-4,4,4-trifluorobutanoic acid
(2S)-2-Amino-5-methyl-hexanoic acid
(2S)-3-(2,3-difluorophenyl)-2-aminopropanoic acid
(2S)-3-(3-Cyanophenyl)-2-aminopropanoic acid
(2S)-3-(4-carboxyphenyl)-2-aminopropanoic acid
(2S)-3-(indol-4-yl)-2-(amino)propanoic acid
(2S)-3-(Triazol-1-yl)-2-(amino)propanoic acid
(2S)-Amino-(1-methyl-1H-indazol-5-yl)acetic acid
(2S)-Amino-2-[3-(Trifluoromethyl)bicyclo[1.1.1]pent-1-yl]acetic acid
(2S)-Pyrrolidin-2-ylacetic acid (beta-homo-P)
(2S,3aS,6aS)-octahydrocyclopenta[b]pyrrole-2-carboxylic acid
(2S,3S)-2-((Amino)methyl)-3-methylpentanoic acid
(2S,3S)-2-[(3R)-3-Amino-2-oxopyrrolidin-1-yl]-3-methylpentanoic acid
(2S,3S)-2-[(3S)-2-oxopiperazin-1-yl]-3-methylpentanoic acid
(2S,4S)-4-fluoroproline ((cis-4-Fluoro)P)
(2S,4S)-4-Trifluoromethyl-pyrrolidine-2-carboxylic acid ((4-CF3)P)
(3R,6R)-1,1-Difluoro-5-azaspiro[2.4]heptane-6-carboxylic acid (enantiomer 1)
(3R,6R)-1,1-Difluoro-5-azaspiro[2.4]heptane-6-carboxylic acid (enantiomer 2)
(4aR,6aR,9S,11aS)-11-oxo-2,3,4,4a,6a,7,8,9,11,11a-decahydro-1H-pyrido[3,2-e]pyrrolo[1,2-a]azepine-9-carboxylic acid
(6S)-5-Azaspiro[2.4]heptane-6-carboxylic acid
(R)-3-Aminoadipic acid
(R)-4-Amino-6-methylheptanoic acid
(R)-Piperidine-3-Carboxylic Acid
(R)-Pyrrolidine-3-Carboxylic Acid
(S)-(1-Piperidin-3-yl)-acetic acid
(S)-(trifluoromethyl)-L-cysteine
(S)-2-(Amino)-1,6-hexanedioic acid (AAD)
(S)-2-Amino-2-cyclobutylacetic acid (Cbg)
(S)-2-Amino-3-ethyl-pentanoic acid
(S)-3-(1-Pyrrolidine-2-yl)-propionic acid
(S)-4-Piperazine-2-carboxylic acid
(S)-Piperidine-3-carboxylic acid
(S)-Pyrrolidine-2-carboxylic acid (beta-P)
[(2R)-4,4-Difluoropyrrolidin-2-yl]acetic acid
1-(Aminomethyl)-cyclopropyl-1-carboxylic acid
1,13-Diamino-4,7,10-trioxatridecan-succinamic acid
12-Amino-4,7,10-trioxadodecanoic acid
14-Amino-3,6,9,12-tetraoxatetradecanoic acid
15-Amino-4,7,10,13-tetraoxa(Pen)tadecanoic acid
17-Amino-3,6,9,12,15-(Pen)taoxaheptadecanoic acid
18-Amino-4,7,10,13,16-(Pen)taoxaoctadecanoic acid
1-Amino-3,6,9,12,15,18,21,24,27-nonaoxatriacontan-30-oic acid
I-Amino-3,6,9,12,15,18,21,24-octaoxaheptacosan-27-oic acid
1-Amino-3,6,9,12,15,18,21-heptaoxatetracosan-24-oic acid
1-Amino-3,6,9,12,15,18-hexaoxahenicosan-21-oic acid
1-Aminocyclobutane-1-carboxylic acid (ACBA)
1-Benzyl-L-histidine (H(1-Bn))
1-Methyl-L-histidine (H(1-Me))
2-(Cyclohexylamino)acetic acid
2,3,3a,4,5,6,7,7a-Octahydroindole-2-carboxylic acid (Oic)
2,5-difluoro-L-phenylalanine
2-[(1S,2S)-1-(amino)-2-methylbutyl]-1,3-oxazole-4-carboxylic acid
2-Amino-1,7-heptanedioic acid
2-Amino-5,5,5-trifluoro-4-methyl-pentanoic acid
2-Amino-7-(tert-butoxy)-7-oxoheptanoic acid
2-Aminoisobutyric acid (Aib)
2-Chloro-L-phenylalanine ((2-Chloro)F)
2-Fluoro-L-phenylalanine ((2-Fluoro)F)
2-Methyl-D-alloisoleucine
2-Methyl-L-phenylalanine ((2-Me)F)
2-Methyl-L-Proline (2-Me)P,
3-(1,3-Benzothiazol-2-yl)-L-alanine ((Bth)A)
3-(Aminomethyl)benzoic acid
3-(Trimethylsilyl)-L-alanine
3-Amino-2,2-dimethylpropionic acid
3-Aminomethylphenylacetic acid
3-Azido-L-Alanine
3-Carboxyphenylalanine
3-Chloro-L-Phenylalanine
3-Chlorophenylglycine ((3-Chloro-Ph)G)
3-Cyano-L-phenylalanine
3-Ethyl-L-Norvaline
3-Fluoro-L-phenylalanine
3-Methyl-L-phenylalanine
4-(3,5-Dimethyl-1,2-oxazol-4-vi)-L-phenylalanine
4-(Aminomethyl)benzoic acid
4-Aminomethylphenylacetic acid
4-Ethyl-L-norleucine
4-Fluoro-Leucine ((4-Fluoro)L)
4-Fluoro-L-phenylalanine ((4-Fluoro)F)
5,5,5-Trifluoro-L-leucine ((Trifluoro)L)
5-azaspiro[2.4]heptane-6-carboxylic acid
6-Aminohexanoic acid (Ahx)
8-Aminocubane-1-carboxylic acid
9-Amino-4,7-dioxanonanoic acid
allo-L-Isoleucine (allo-I)
allo-L-Threonine (allo-T)
Aminocyclobutanecarboxylic acid (ACBC)
Aminoisobutyric acid (Aib)
beta-Alanine (beta-A)
Cyclohexylalanine (Cha)
D-2-Chlorophenylalanine
D-beta-Proline
D-cyclohexylalanine
D-Hydroxyproline
D-N-Methylalanine
Gamma-Aminobutyric acid (Gamma-Abu)
Hydroxyproline (Hyp)
Iminodiacetic acid
L-Homoserine (hSer)
L-1-Napthylalanine (1-Nal)
L-2,3-Diaminopropionic acid (Dap)
L-2,4-Diaminobutyric acid (Dab)
L-2,6-Difluorophenylalanine
L-2-Amino-4-cyanobutyric acid
L-2-Aminobutyric acid (Abu)
L-2-Bromophenylalanine ((2-Bromo)F)
L-2-Napthylalanine (2-Nal)
L-2-Pyridylalanine (2-Pal)
L-2-Thienylalanine
L-3-Bromophenylalanine ((3-Bromo)F)
L-3-Methylhistidine (H(3-Me))
L-3-Pyridylalanine (3-Pal)
L-4,4-difluoroproline ((Difluoro)P)
L-4-Aminophenylalanine ((4-Amino)F)
L-4-Bromophenylalanine
L-4-Pyridylalanine
L-Citrulline (Cit)
L-Cyclobutylalanine (Cba)
L-Cyclobutylglycind
L-Cyclohexylalanine
L-Cyclohexylglycine
L-Cyclohexylglycine (Chg)
L-Cyclopentylalanine
L-cyclopentylalanine (Cpa)
L-Cyclopentylglycine (Cpg)
L-Cyclopropylmethylalanine
L-Difluoromethylalanine
L-Dihydroorotic acid (Hoo)
L-Homocysteine
L-Hydroxyproline (Hyp)
L-Methionine-L-sulfoxide
L-Methionine-sulfone
L-N,N-Dimethylalanine ((N,N-diMe)A)
L-N-Methylalanine
L-N-Methylcysteine ((N-Me)C)
L-N-Methylisoleucine ((N-Me)I)
L-N-Methylphenylalanine ((N-Me)F)
L-Norleucine (Nle)
L-Norvaline (Nva)
L-Ornithine (Orn)
L-Penicillamine (Pen)
L-Phenylglycine (Phg)
L-Pipecolic acid (Pip)
L-Propargylglycine
L-Pyroglutamic acid (Pyr)
L-tert-Butylalanine ((tBu)A)
L-tert-Butylglycine ((tBu)G)
L-trans-3-Hydroxyproline ((3S—OH)P)
L-Trifluoromethylalanine
Morpholine-3-carboxylic
N(5)-methyl-L-arginine ((Me)R)
N-e-Isopropyl-L-lysine
N-Methyl-Alanine (N-Me)A
N-Methyl-Glycine ((N-Me)G)
N-Phenylglycine ((N-Ph)G)
Palmitic acid (Palm)
rel-(1R,2S)-2-Amino-1-cyclopentanecarboxylic acid (ACPC)
rel-(1R,3R,5R,6R)-6-(Trifluoromethyl)-2-azabicyclo[3.1.0]hexane-3-carboxylic acid
rel-(1R,3S)-3-[(Amino)methyl]cyclohexanecarboxylic acid
rel-(3R,6R)-1,1-difluoro-5-azaspiro[2.4]heptane-6-carboxylic acid (enantiomer 1)
rel-(3R,6R)-1,1-difluoro-5-azaspiro[2.4]heptane-6-carboxylic acid (enantiomer 2)
S-2-amino-3-ethyl-pentanoic acid
S-3-1-Pyrrolidin-2-yl-propionic acid
Tranexamic acid (Tranexamic)
trans-2-(3-(Amino)cyclohexyl)acetic acid
trans-4-Fluoroproline ((trans-4-Fluoro)P)
3-Amino-3-methylbutyric acid

More preferred unnatural amino acid are selected from a list consisting of N-Methyl-Alanine (N-Me)A, N-Methyl-Glycine ((N-Me)G), (1R,3S,4S)-2-azabicyclo[2.2.1]heptane-3-carboxylic acid, L-3-Bromophenylalanine ((3-Bromo)F), L-N,N-Dimethylalanine ((N,N-diMe)A), N,N-Dimethylglycine ((N,NdiMe)G),N-Phenylglycine((N-Ph)G), (R)-Piperidine-3-carboxylicacid(S)-Piperidine-3-carboxylicacid, L-tert-Butylalanine ((tBu)A), L-2-Pyridylalanine (2-Pal), L-3-Pyridylalanine (3-Pal), L-4-Pyridylalanine (4-Pal), 3-(Aminomethyl)benzoic acid, 3-Amino-2,2-dimethylpropionic acid, 3-Amino-3-methylbutric acid, 4-(Aminomethyl)benzoic acid, L-2-Aminobutyric acid (Abu), 1-Aminocyclobutane-1-carboxylic acid (ACBA), 6-Aminohexanoic acid (Ahx), 2-Aminoisobutyric acid (Aib), L-2-Thienylalanine (beta-2-thienylalanine), beta-Alanine (beta-A), beta-Proline (beta-P), L-Citrulline (Cit), L-2,4-Diaminobutyric acid (Dab), L-2,3-Diaminopropionic acid (Dap), Gamma-Aminobutyric acid (Gamma-Abu), L-3-Methylhistidine (3-Me)H), L-Dihydroorotic acid (Hoo), L-Norleucine (Nle), N-Methyl-L-proline ((NMe)P), L-Norvaline (Nva), L-Omithine (Orn), L-Pipecolic acid (Pip), (2S)-2[(Amino)-2-(tetrahydro-2H-pyran-4-yl)]acetic acid, 2,3,3a,4,5,6,7,7a-Octahydroindole-2-carboxylic acid (Oic), L-N-Methylcysteine ((N-Me)C), N(5)-methyl-L-arginine ((Me)R), L-Penicillamine (Pen) and Tranexamic acid (Tranexamic). Most preferred unnatural amino acid arm selected from a list consisting of N-Methyl-L-Alanine (N-Me)A, N-Methyl-Glycine ((N-Me)G), L-Norleucine (Nle), L-Norvaline (Nva), L-Omithine (Orn), N(5)-methyl-L-arginine ((Me)R), L-tert-Butylalanine ((tBu)A), 2,3,3a,4,5,6,7,7a-Octahydroindole-2-carboxylic acid (Oic), L-N-Methylcysteine ((N-Me)C) and L-Penicillamine (Pen).

It should be further understood, that a peptide according to the invention can contain one or more chemical groups which ae no amino acid under the definition of the present invention. These chemical groups can be present at the N- and/or C-terminal ends of a peptide and are represented by formula X⁰ and X¹⁵. It should be understood that all amino acids and chemical groups of the peptides of the present invention are connected via peptide (amide) bonds. Generally peptides are formed by linking α-amino and carboxy groups of α-amino acids, which are then linked by α-peptide bonds. According to the present invention a peptide bond can be formed by any carboxyl- and amino group being present in a respective natural or unnatural amino acid. For example, α-amino acids which contain a second amino group in addition to the α-amino group (e.g. L-lysine) or α-amino acids which, in addition to the α-carboxy group, contain a second carboxy group, (eg. L-aspartic acid and L-glutamic acid) can be connected via the additional amino- or carboxy group.

In accordance with the understanding of a person skilled in the art, the peptide sequences disclosed herein represent sequences of amino acids, which are connected via α-peptide bonds.

In accordance with the understanding of a person skilled in the art, the peptide sequences disclosed herein are shown proceeding from left to right, with the left end of the sequence being the “N-terminus” (“amino terminus”, “N-terminal end”) of the peptide and the right end of the sequence being the “C-terminus” (“carboxy terminus”, “C-terminal end”) of the peptide. This terminology N-terminus (amino terminus, N-terminal end)” applies irrespective of whether the peptide actually contains an amino group at the N-terminus. This terminology C-terminus (carboxy terminus, C-terminal end) applies irrespective of whether the peptide actually contains a carboxy group at the C-terminus. The term “terminal amino group” refers to any amino group present at the N-terminus. The term “terminal carboxyl group” refers to any carboxyl group present at the C-terminus.

According to the present invention the N-terminus can be formed by X, in case R¹ is absent. Alternatively the N-terminus can be formed by R¹.

In the present invention the names of naturally occurring and non-naturally occurring aminoacyl residues used herein are preferably following the naming conventions suggested by the IUPAC Commission on the Nomenclature of Organic Chemistry and the IUPAC-IUB Commission on Biochemical Nomenclature as set out in Nomenclature of α-Amino Acids (Recommendations. 1974). Biochemistry. 14(2). (1975).

In the present specification naturally occurring proteinogenic amino acids are usually designated by their conventional single-letter abbreviations. Alternatively, they can also be referred to by their three-letter abbreviations (e.g. in particular in the sequence listings) or by their full name as shown in Table 2 below: Table 2: Standard Abbreviations for Natural Amino Acids

TABLE 2
Standard Abbreviations for Natural Amino Acids

| 3-Letter | 1-Letter | Amino Acid |
|---|---|---|
| Ala | A | Alanine |
| Arg | R | Arginine |
| Asn | N | Asparagine |
| Asp | D | Aspartic acid |
| Cys | C | Cysteine |
| Glu | E | Glutamic acid |
| Gln | Q | Glutamine |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Ser | S | Serine |
| Thr | T | Threonine |
| Trp | W | Tryptophan |
| Tyr | Y | Tyrosine |
| Val | V | Valine |

In the case of non-proteinogenic or non-naturally occurring amino acids, unless they are referred to by their full name (e.g. omithine, etc.), frequently employed three- to six-character codes are employed for residues thereof, including those abbreviations as indicated in the abbreviation list below (Table 3).

The term “L-amino acid” as used herein refers to the “L” isomeric form of an amino acid, and conversely the term “D-amino acid” refers to the “D” isomeric form of an amino acid. It is further a conventional manner to indicate the L-amino acid with capital letters such as Ala/A, Arg/R, etc. and the D-amino acid with small letters such as ala/a, arg/r, etc.

The three-letter code in the form as indicated in Table 2 above, i.e. Ala, Arg, Asn etc. and as generally used in the present specification, shall generally comprise the D- and L-form as well as homo- and nor-forms, unless explicitly indicated otherwise. The prefix “nor” refers to a structural analog that can be derived from a parent compound by the removal of one carbon atom along with the accompanying hydrogen atoms. The prefix “homo” indicates the next higher member in a homologous series. A reference to a specific isomeric form will be indicated by the capital prefix L- or D- as described above (e.g. D-Arg, L-Arg etc.). A specific reference to homo- or nor-forms will accordingly be explicitly indicated by a respective prefix (e.g. homo-Arg, homo-R, nor-Arg, nor-R, homo-Cys, homo-C etc.).

The one-letter code in the form as indicated in Table 2 above, i.e. A, R, N etc. and as generally used in the present specification, shall generally comprise the D- and L-form as well as homo- and nor-forms.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group. Particularly preferred is methyl, ethyl, n-propyl. Most preferred is methyl.

The term “C₁-C₂₀-alkyl” means a linear or branched, saturated, monovalent hydrocarbon group having 1, to 20 carbon atoms, e.g. a methyl, ethyl, propyl, isopropyl, butyl, sec-butyl isobutyl, tert-butyl or pentyl, isopentyl, hexyl, isohexyl, heptyl, isoheptyl, octyl and isooctyl, nonyl, decyl, dodecyl or eicosyl.

The term “C₁-C₄-alkylene” means a straight-chain or branched hydrocarbon bridge having 1 to 4 carbon atoms, e.g. methylene, ethylene, propylene, (α-methylethylene, β-methylethylene, α-ethylethylene, β-ethylethylene, butylene, α-methylpropylene, β-methylpropylene and γ-methylpropylene.

The term “C₁-C₆-alkylene” means a straight-chain or branched hydrocarbon bridge having 1 to 6 carbon atoms. e.g. methylene, ethylene, propylene, (α-methylethylene, β-methylethylene, α-ethylethylene, β-ethylethylene, butylene, α-methylpropylene, β-methylpropylene, γ-methylpropylene, α-ethylpropylene, β-ethylpropylene, γ-ethylpropylene, pentylene and hexylene.

The term “C₃-C₈-cycloalkyl” means a saturated hydrocarbon ring which contains 3, 4, 5, 6, 7 or 8 carbon atoms. Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, a bicyclic hydrocarbon ring, e.g. a bicyclo[4.2.0]octyl or octahydropentalenyl, or a bridged or caged saturated ring groups such as nor-borane or adamantane, and cubane.

The term “C₃-C₇-heterocycloalkyl” means a saturated heterocycle with 4, 5, 6 or 7 which contains one or two identical or different ring heteroatoms from the series N, O and S, it being possible for said heterocycloalkyl group to be attached to the rest of the molecule via any one of the carbon atoms or, if present, a nitrogen atom. Said C₃-C₇-heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanvl, 1,2-oxazolidinyl, 1,3-oxazolidinyl or 1,3-thiazolidinyl, for example; or a 6 membered ring, such as tetrahydropyranyl, tetrahydrothiopyranyl, piperidinyl, morpholinyl, dithianyl, thiomorpholinyl, piperazinyl, hexahydropyrimidinyl, 1,3-dioxanyl, 1,4-dioxanyl or 1,2-oxazinanyl, for example, or a 7 membered ring, such as azepanyl, 1,4-diazepanyl or 1,4-oxazepanyl, for example.

The term “aryl” means an unsaturated or partially unsaturated cycle having 6 to 10 carbon atoms. Preferred aryl radicals are phenyl and naphthyl.

The term “heteroaryl” means a monovalent, monocyclic, bicyclic or tricyclic aromatic ring having 5, 6, 8, 9, 10, 11, 12, 13 or 14 ring atoms (a “5 to 14 membered heteroaryl” group), particularly 5, 6, 9 or 10 ring atoms, which contains at least one ring heteroatom and optionally one, two or three further ring heteroatoms from the series: N, O and/or S. and which is bound via a ring carbon atom or optionally via a ring nitrogen atom (if allowed by valency). Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrimidinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridine-2-yl, pyridine-3-yl and pyridine-4-yl; or the term thienyl includes thien-2 yl- and thien-3-yl.

Among sequences disclosed herein are sequences incorporating either an “—OH” moiety or an “—NH₂” moiety at the carboxy terminus (C-terminus) of the sequence. An “—OH” or an “—NH₂” moiety at the C-terminus of the sequence indicates a hydroxy group or an amino group, corresponding to the presence of a carboxy group or an amido (—(C═O)—NH₂) group at the C-terminus, respectively. In each sequence of the invention, a C-terminal “—OH” moiety may be substituted for a C-terminal “—NH₂” moiety, which is also referred to as “amidated C-terminus” in the present invention, and vice-versa. However, among said alternatives a C-terminal “—OH” moiety is preferred.

The term “acetylated” (also abbreviated “Ac”) refers to an acetyl protection of the N-terminal moiety through acetylation of the N-terminus of a peptide (N-terminus of the peptide is acetylated).

Preferred salts in the context of the present invention are physiologically acceptable salts of the compounds according to the invention. Also encompassed are salts which are not themselves suitable for pharmaceutical applications but can be used, for example, for the isolation, purification or storage of the compounds of the invention.

A suitable pharmaceutically acceptable salt of the compounds of the present invention may be, for example, an acid-addition salt of a compound of the present invention bearing a sufficiently basic nitrogen atom in a chain or in a ring, such as an acid-addition salt with an inorganic acid, or “mineral acid”, such as hydrochloric acid, hydrobromic acid, hydroiodic acid, sulfuric acid, sulfamic acid, bisulfuric acid, phosphoric acid or nitric acid, for example, or with an organic acid such as formic acid, acetic acid, acetoacetic acid, pyruvic acid, trifluoroacetic acid, propionic acid, butyric acid, hexanoic acid, heptanoic acid, undecanoic acid, lauric acid, benzoic acid, salicylic acid, 2-(4-hydroxybenzoyl)benzoic acid, camphoric acid, cinnamic acid, cyclopentanepropionic acid, digluconic acid, 3-hydroxy-2-naphthoic acid, nicotinic acid, pamoic acid, pectinic acid, 3-phenylpropionic acid, pivalic acid, 2-hydroxyethanesulfonic acid, itaconic acid, trifluoromethanesulfonic acid, dodecylsulfuric acid, ethanesulfonic acid, benzenesulfonic acid, para-toluenesulfonic acid, methanesulfonic acid, 2-naphthalenesulfonic acid, naphthalenedisulfonic acid, camphorsulfonic acid, citric acid, tartaric acid, stearic acid, lactic acid, oxalic acid, malonic acid, succinic acid, malic acid, adipic acid, alginic acid, maleic acid, fumaric acid, D-gluconic acid, mandelic acid, ascorbic acid, glucoheptanoic acid, glycerophosphoric acid, aspartic acid, sulfosalicylic acid or thiocyanic acid, for example.

Further, another suitable pharmaceutically acceptable salt of a sufficiently acidic compound of the present invention is an alkali metal salt, for example a sodium or potassium salt, an alkaline earth metal salt, for example a calcium, magnesium or strontium salt, or an aluminum or zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methylglucamine, N,N-dimethylglucamine, N-ethylglucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognize that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae relating to salts, such as “hydrochloride”, “trifluoroacetate”, “sodium salt”, or “x HCl”, “x CF₃COOH”, “x Na”, for example, mean a salt form, the stoichiometry of this salt not being specified. This applies analogously to cases in which synthesis intermediates or example compounds or salts thereof have been obtained as solvates, for example hydrates, by the preparation and/or purification processes described.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. It is possible to isolate the stereoisomerically homogeneous constituents from such mixtures of enantiomers and/or diastereomers in a known manner. Preference is given to employing chromatographic methods for this purpose, especially HPLC chromatography on achiral or chiral separation phases. In the case of carboxylic acids as intermediates or end products, separation is alternatively also possible via diastereomeric salts using chiral amine bases.

In the context of the present invention, the term “enantiomerically pure” is understood to the effect that the compound in question with respect to the absolute configuration of the chiral centers is present in an enantiomeric excess of more than 95%, preferably more than 98%. The enantiomeric excess, ee, is calculated here by evaluating an HPLC analysis chromatogram on a chiral phase using the formula below:

If the compounds of the invention can occur in tautomeric forms, the present invention encompasses all the tautomeric forms.

The present invention also encompasses all suitable isotopic variants of the compounds of the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature (“unnatural fraction”). The expression “unnatural fraction” is understood to mean a fraction of such an isotope higher than its natural frequency. The natural frequencies of isotopes to be employed in this connection can be found in “Isotopic Compositions of the Elements 1997”, Pure Appl. Chem., 70(1), 217-235, 1998. Examples of isotopes which can be incorporated into a compound according to the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to the comparatively easy preparability and detectability, especially compounds labeled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds of the invention may therefore possibly also constitute a preferred embodiment of the present invention. With regard to the treatment and/or prophylaxis of the disorders specified here, the isotopic variant(s) of the compounds of the general formula (I) preferably contain deuterium (“deuterium-containing compounds of the general formula (I)”). Isotopic variants of the compounds of the general formula (I) into which one or more radioactive isotopes such as ³H or ¹⁴C have been incorporated are beneficial, for example, in medicament and/or substrate tissue distribution studies. Because of their easy incorporability and detectability, these isotopes are particularly preferred. It is possible to incorporate positron-emitting isotopes such as ¹⁸F or ¹¹C into a compound of the general formula (I). These isotopic variants of the compounds of the general formula (I) are suitable for use in in vivo imaging applications. Deuterium-containing and ¹³C-containing compounds of the general formula (I) can be used within the scope of preclinical or clinical studies in mass spectrometry analyses (H. J. Leis et al., Curr. Org. Chem., 1998, 2, 131). Isotopic variants of the compounds of the invention can be prepared by commonly used processes known to those skilled in the art, for example by the methods described further down and the procedures described in the working examples, by using corresponding isotopic modifications of the respective reagents and/or starting compounds.

Isotopic variants of the compounds of the general formula (I) can generally be prepared by processes known to those skilled in the art as described in the schemes and/or examples described here, by replacing a reagent with an isotopic variant of the reagent, preferably a deuterium-containing reagent. According to the deuteration sites desired, it is possible in some cases to incorporate deuterium from D₂O either directly into the compounds or into reagents which can be used for the synthesis of such compounds (Esaki et al., Tetrahedron, 2006, 62, 10954; Esaki et al., Chem. Eur. J., 2007, 13, 4052). A photochemical deuteration and tritiation method has also been described (Y. Y. Loh et al., Science 10.1126/science.aap9674 (2017). Another useful reagent for incorporation of deuterium into molecules is deuterium gas. A rapid route for incorporation of deuterium is the catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron, 2011, 52, 3865). For direct exchange of hydrogen for deuterium in hydrocarbons containing functional groups, it is also possible to use metal catalysts (i.e. Pd, Pt and Rh) in the presence of deuterium gas (J. G. Atkinson et al., U.S. Pat. No. 3,966,781). Various deuterated reagents and synthesis units are commercially available from companies like, for example, C/D/N Isotopes, Quebec, Canada; Cambridge Isotope Laboratories Inc., Andover, MA. USA; and CombiPhos Catalysts, Inc., Princeton, NJ, USA. Further information relating to the prior art with regard to deuterium-hydrogen exchange can be found, for example, in Hanzlik et al., J. Org. Chem., 1990, 55, 3992-3997; R. P. Hanzlik et al., Biochem. Bio-phys. Res. Commun., 1989, 160, 844; P. J. Reider et al., J. Org. Chem., 1987, 52, 3326-3334: M. Jarman et al., Carcinogenesis, 1993, 16(4), 683-688; J. Atzrodt et al., Angew. Chem., Int. Ed. 2007, 46, 7744; K. Matoishi et al., 2000, J. Chem. Soc. Chem. Commun., 1519-1520; K. Kassahun et al., WO 2012/112363.

The term “deuterium-containing compound of the general formula (I)” is defined as a compound of the general formula (I) in which one or more hydrogen atoms have been replaced by one or more deuterium atoms and in which the frequency of deuterium in every deuterated position in the compound of the general formula (I) is higher than the natural frequency of deuterium, which is about 0.015%. More particularly, in a deuterium-containing compound of the general formula (I), the frequency of deuterium in every deuterated position in the compound of the general formula (I) is higher than 10%, 20%, 30%, 40%, 50%, 60%, 70% or 80%, preferably higher than 90%, 95%, 96% or 97%, even further preferably higher than 98% or 99%, in this position or these positions. It will be apparent that the frequency of deuterium in every deuterated position is independent of the frequency of deuterium in other deuterated positions.

The selective incorporation of one or more deuterium atoms into a compound of the general formula (I) can alter the physicochemical properties (for example acidity [A. Streitwieser et al., J. Am. Chem. Soc., 1963, 85, 2759; C. L. Perrin et al., J. Am. Chem. Soc., 2007, 129, 4490], basicity [C. L. Perrin, et al., J. Am. Chem. Soc., 2003, 125, 15008; C. L. Perrin in Advances in Physical Organic Chemistry, 44, 144; C. L. Perrin et al., J. Am. Chem. Soc., 2005, 127, 9641], lipophilicity [B. Testa et al., Int. J. Pharm., 1984, 19(3), 271]) and/or the metabolic profile of the molecule, and cause changes in the ratio of parent compound to metabolites or the amounts of metabolites formed. Such changes may lead to particular therapeutic benefits and therefore be preferable under particular circumstances. Reduced rates of metabolism and metabolic switching, where the ratio of metabolites is changed, have been reported (D. J. Kushner et al., Can. J. Physiol. Pharmacol., 1999, 77, 79; A. E. Mutlib et al., Toxicol. Appl. Pharmacol., 2000, 169, 102). These changes in the exposure to parent compound and metabolites can have important consequences with respect to the pharmacodynamics, tolerability and efficacy of a deuterium-containing compound of the general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26.410; Uetrecht et al., Chemical Research in Toxicology, 2008, 21, 9, 1862; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. Indiplon (A. J. Morales et al., Abstract 285, The 15^{th} North American Meeting of the International Society of Xenobiotics, San Diego, CA, Oct. 12-16, 2008). ML-337 (C. J. Wenthur et al., J. Med. Chem., 2013, 56, 5208), and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch. Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

The present invention additionally also encompasses prodrugs of the compounds of the invention. The term “prodrugs” refers here to compounds which may themselves be biologically active or inactive, but are converted while present in the body, for example by a metabolic or hydrolytic route, to compounds of the invention.

When radicals in the compounds of the invention are substituted, the radicals may be mono- or polysubstituted, unless specified otherwise. In the context of the present invention, all radicals which occur more than once are defined independently of one another. When radicals in the compounds of the invention are substituted, the radicals may be mono- or polysubstituted, unless specified otherwise. Substitution by one substituent or by two identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

Preference is given in the context of the present invention to compounds of the formula (I) in which
R¹ is absent
   or
   represents 6-Carboxytetramethylrhodamine (Tam), ##C(O)R³, C₈-C₂₀ fatty acid or the sequence R⁴GFLG ##, R⁴—C═N—NH-##, R⁴—S—S-##, R⁴—N═N-##, R⁴-Valin-Citrullin-##, R⁴—C(O)O-## or R⁴NH—C(O)O-##,
      wherein
      ## marks the attachment to the terminal amino group of X¹,
      R³ represents C₁-C₆-alkylene, aryl, heteroaryl, C₃-C₈-cycloalkyl or C₃-C₇-heterocycloalkyl,
         wherein C₁-C₆-alkylene is up to trisubstituted identically or differently by a radical selected from the group consisting of hydroxyl, methoxy, ethoxy, carboxy, amino and halogen,
         wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
      R⁴ represents

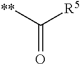

wherein
R⁵ represents C₁-C₆-alkylene, aryl, heteroaryl, C₃-C₈-cycloalkyl or C₃-C₇-heterocycloalkyl,
wherein C₁-C₆-alkylene is up to trisubstituted identically or differently by a radical selected from the group consisting of hydroxyl, methoxy, ethoxy, carboxy, amino and halogen,
wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen, or
represents a group of the formula (IIIa)

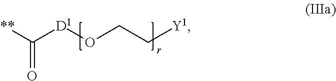

wherein
** marks the attachment to a nitrogen atom,
D¹ is C₁-C₄-alkylene,
Y¹ is selected from the group consisting of hydroxyl, methoxy, ethoxy, carboxy, carboxamide or amino,
   wherein amino might be substituted with 6-carboxytetramethyirhodamine (Tam) via an amide bond,
and
r represents an integer of from 2 to 15,
R² represents a group of the formula (II)

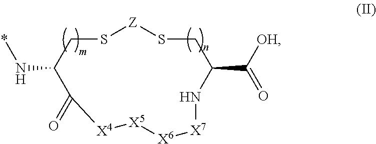

wherein
* represents the attachment to the carbonyl atom of the carboxy group of X³,
Z represents a bond or —CH₂—,
m represents 1 or 2,
n represents 1 or 2,
X¹ represents a natural amino acid selected from a list consisting of L, I, F, H, M, W, Y or y or an unnatural amino acid selected from a list consisting of L-Norleucine (Nle), 2,3,3a,4,5,6,7,7a-Octahydroindole-2-carboxylic acid (Oic), 4-Bromophenylalanine ((4-Bromo)F), 2,5-Difluorophenylalanine ((2,5-Difluoro)F), 2-Chlorophenylalanine ((2-Chloro)F), 3-Chlorophenylalanine ((3-Chloro)F), 4-Chlorophenylalanine ((4-Chloro)F), 2-Bromophenylalanine ((2-Bromo)F), 3-Bromophenylalanine ((3-Bromo)F), 4-Bromophenylalanine ((4-Bromo)F), 2-Fluorophenylalanine ((2-Fluoro)F), 3-Fluorophenylalanine ((3-Fluoro)F), 4-Fluorophenylalanine ((4-Fluoro)F), (2,5-difluoro-phenylalanine, 2-Methyl-phenylalanine ((2-Me)F), 3-Methyl-phenylalanine ((3-Me)F), 4-Methylphenylalanine ((4-Me)F), (2S)-3-(2,3-difluorophenyl)-2-aminopropanoic acid, Phenylglycine (Phg)N-Phenylglycine ((N-Ph)G), 3-Chlorophenylglycine ((3-Chloro-Ph)G), 3-(1,3-Benzothiazol-2-yl)-alanine 1-Benzyl-histidine (H(1-Bn)), 1-Methyl-histidine (H(1-Me)), 3-Methylhistidine (3-Me)H), 2-Pyridylalanine (2-Pal), 3-Pyridylalanine (3-Pal), 4-Pyridylalanine (4-Pal), 3-(Aminomethyl)benzoic acid, 1-Napthylalanine (1-Nal), 2-Napthylalanine (2-Nal), (2R)-Amino-(1-methyl-1H-indazol-5-yl)acetic acid and (2S)-3-(indol-4-yl)-2-(amino)propanoic acid, whereas any natural amino acid and/or unnatural amino acid from that list can be in D- or L-stereoconfiguration,
X² represents a natural amino acid selected from a list consisting of L, I, F, H, M, W or Y or an unnatural amino acid selected from a list consisting of L-Norleucine (Nle), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Napthylalanine (1-Nal), L-2-Napthylalanine (2-Nal), (2R)-Amino-(1-methyl-1H-indazol-5-yl)acetic acid and (2S)-3-(indol-4-yl)-2-(amino)propanoic acid,
X³ represents the natural amino acid P, or an unnatural amino acid selected from a list consisting of 2,3,3a,4,5,6,7,7a-Octahydroindole-2-carboxylic acid (Oic), L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline ((3S—OH)P, L-Pipecolic acid (Pip), (1R,3S,5R)-2-azabicyclo[3.1.0]hexane-3-carboxylic acid, (6S)-5-Azaspiro-[2.4]heptane-6-carboxylic acid, rel-(1R,3R,5R,6R)-6-(trifluoromethyl)-2-azabicyclo[3.1.0]hexane-3-carboxylic acid, (2S)-2-Amino-4,4,4-trifluorobutanoic acid, (2S,3aS,6aS)-octahydrocyclopenta[b]^{¬}pyrrole-2-carboxylic acid, trans-4-fluoroproline ((trans-4-Fluoro)P), (2S,4S)-4-fluoroproline ((cis-4-Fluoro)P), L-4,4-difluoroproline ((Difluoro)P), rel-(3R,6R)-1,1-difluoro-5-azaspiro[2.4]heptane-6-carboxylic acid (enantiomer 1) and rel-(3R,6R)-1,1-difluoro-5-azaspiro[2.4]heptane-6-carboxylic acid (enantiomer 2),
X⁴ represents any natural amino acid or an unnatural amino acid, whereas any natural amino acid and/or unnatural amino acid can be in D- or L-stereoconfiguration,
X⁵ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chloro-L-phenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Naphthylalanine (1-Nal), L-2-Naphthylalanine (2-Nal), (2R)-Amino-(1-methyl-1H-indazol-5-yl)acetic acid and (2S)-3-(indol-4-yl)-2-(amino)propanoic acid,
X⁶ represents any natural amino acid or an unnatural amino acid, whereas any natural amino acid and/or unnatural amino acid can be in D- or L-stereoconfiguration,
   wherein any natural amino acid or an unnatural amino acid bearing an amino group might be substituted with 6-Carboxytetramethylrhodamine (Tam) or ##C(O)R³,
      wherein
      ## marks the attachment to the terminal amino group of X¹,
      R³ represents C₁-C₆-alkylene, aryl, heteroaryl. C₃-C₈-cycloalkyl or C₃-C₇-heterocycloalkyl,
      wherein C₁-C₆-alkylene is up to trisubstituted identically or differently by a radical selected from the group consisting of hydroxyl, methoxy, ethoxy, carboxy, amino and halogen,
      wherein aryl, heteroaryl, C₃-C₈-cycloalkyl and C3-C₁-heterocycloalkyl can be up to trisubstituted identically or differently by a radical selected from the group of C₁-C₄-alkyl, hydroxyl, methoxy, ethoxy, carbonyl, carboxy, amino and halogen,
X⁷ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chloro-L-phenylglycine ((3-Chloro-Ph)G), 3-(1,3-Benzothiazol-2-yl)-L-alanine 1-Benzyl-L-histidine (H(1-Bn)), I-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Naphthylalanine (1-Nal), L-2-Naphthylalanine (2-Nal), (2R)-Amino-(1-methyl-1H-indazol-5-yl)acetic acid and (2S)-3-(indol-4-yl)-2-(amino)propanoic acid,
or a pharmaceutically acceptable salt, hydrate, solvate or solvate of the salt thereof,
   with the proviso, that compound YFP[cQFAFC] is excluded.

Further preference is given in the context of the present invention to compounds of the formula (I) in which
R¹ is absent
   or
   represents 6-Carboxytetramethylrhodamine (Tam), ##C(O)R³ or the sequence R⁴GFLG##,
      wherein
      ## marks the attachment to the terminal amino group of X¹,
      R³ represents C₁-C₄-alkylene,
         wherein C₁-C₄-alkylene is up to trisubstituted identically or differently by a radical selected from the group consisting of hydroxyl, methoxy, ethoxy, carboxy, amino, fluoro and chloro,
      R⁴ represents

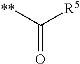

wherein
R⁵ represents C₁-C₄-alkylene,
wherein C₁-C₄-alkylene is up to trisubstituted identically or differently by a radical selected from the group consisting of hydroxyl, methoxy, ethoxy, carboxy, amino, chloro and fluoro,
or
represents a group of the formula (IIIa)

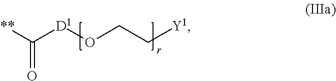

wherein
** marks the attachment to a nitrogen atom,
D¹ is C₁-C₄-alkylene,
Y¹ is selected from the group consisting of hydroxyl, methoxy, ethoxy, carboxy, carboxamide or amino,
   wherein amino might be substituted with 6-carboxytetramethylrhodamine (Tam) via an amide bond,
and
r represents an integer of from 2 to 6,
R² represents a group of the formula (II)

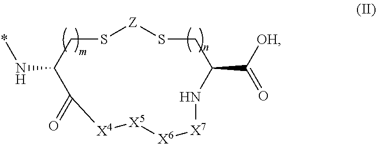

wherein
* represents the attachment to the carbonyl atom of the carboxy group of X³,
Z represents a bond or —CH₂—,
m represents 1 or 2,
n represents 1 or 2,
X¹ represents a natural amino acid selected from a list consisting of L, I, F, H, M, W, Y or y or an unnatural amino acid selected from a list consisting of L-Norleucine (Nle), 2,3,3a,4,5,6,7,7a-Octahydroindole-2-carboxylic acid (Oic), 4-Bromophenylalanine ((4-Bromo)F), 2,5-Difluorophenylalanine ((2,5-Difluoro)F), 2-Chlorophenylalanine ((2-Chloro)F), 3-Chlorophenylalanine ((3-Chloro)F), 4-Chlorophenylalanine ((4-Chloro)F), 2-Bromophenylalanine ((2-Bromo)F), 3-Bromophenylalanine ((3-Bromo)F), 4-Bromophenylalanine ((4-Bromo)F), 2-Fluorophenylalanine ((2-Fluoro)F), 3-Fluorophenylalanine ((3-Fluoro)F), 4-Fluorophenylalanine ((4-Fluoro)F), (2,5-difluoro-phenylalanine, 2-Methyl-phenylalanine ((2-Me)F), 3-Methyl-phenylalanine ((3-Me)F), 4-Methylphenylalanine ((4-Me)F), 1-Benzyl-histidine (H(1-Bn)), 1-Methyl-histidine (H(1-Me)), 3-Methylhistidine (3-Me)H), 2-Pyridylalanine (2-Pal), 3-Pyridylalanine (3-Pal), 4-Pyridylalanine (4-Pal), whereas any natural amino acid and/or unnatural amino acid from that list can be in D- or L-stereoconfiguration.
X² represents a natural amino acid selected from a list consisting of L, I, F, H, M, W or Y or an unnatural amino acid selected from a list consisting of L-Norleucine (Nle), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), I-Benzyl-L-histidine (H(1-Bn)), I-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal),
X³ represents the natural amino acid P, or an unnatural amino acid selected from a list consisting of L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline ((3S—OH)P, (1R3S,5R)-2-azabicyclo[3.1.0]hexane-3-carboxylic acid, (6S)-5-Aza^{¬}spiro^{¬}[2.4]heptane-6-carboxylic acid, rel-(1R,3R,5R,6R)-6-(trifluoromethyl)-2-azabicyclo[3.1.0]hexane-3-carboxylic acid, (2S)-2-Amino-4,4,4-trifluorobutanoic acid, (2S,3aS,6aS)-octahydrocyclopenta[b]^{¬}pyrrole-2-carboxylic acid, (2S,4S)-4-fluoroproline ((cis-4-Fluoro)P), L-4,4-difluoroproline ((Difluoro)P),
X⁴ represents any natural amino acid, whereas any natural amino acid can be in D- or L-stereoconfiguration,
X⁵ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic). L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal),
X⁶ represents any natural amino acid, whereas any natural amino acid can be in D- or L-stereoconfiguration,
   wherein the amino group of Lysin might be substituted with 6-Carboxytetramethylrhodamine (Tam) or ##C(O)R³,
      wherein
      ## marks the attachment to the terminal amino group of X¹,
      R³ represents C₁-C₆-alkylene, aryl, heteroaryl, C₃-C₈-cycloalkyl or C₃-C₇-heterocycloalkyl,
      wherein C₁-C₆-alkylene is up to trisubstituted identically or differently by a radical selected from the group consisting of hydroxyl, methoxy, ethoxy, carboxy, amino and halogen,
      wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
X⁷ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal),
or a pharmaceutically acceptable salt, hydrate, solvate or solvate of the salt thereof,
   with the proviso, that compound YFP[cQFAFC] is excluded.

Further preference is given in the context of the present invention to compounds of the formula (I) in which
R¹ is absent
   or
   represents 6-Carboxytetramethylrhodamine (Tam) or the sequence R⁴GFLG ##,
      wherein
      ## marks the attachment to the terminal amino group of X¹,
      R⁴ represents

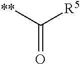

wherein
R⁵ represents methyl or ethyl,
or
represents a group of the formula (IIIa)

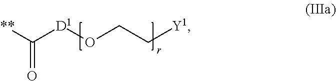

wherein
** marks the attachment to a nitrogen atom,
D¹ is C₁-C₄-alkylene,
Y¹ is selected from the group consisting of hydroxyl, methoxy, ethoxy, carboxy, carboxamide or amino,
   wherein amino might be substituted with 6-carboxytetramethylrhodamine (Tam) via an amide bond.
and
r represents an integer of from 2 to 4.
R² represents a group of the formula (II)

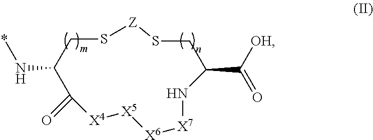

wherein
represents the attachment to the carbonyl atom of the carboxy group of X³,
Z represents a bond or —CH₂—,
m represents 1 or 2,
n represents 1 or 2,
X¹ represents a natural amino acid selected from a list consisting of F, H, Y or y, whereas any amino acid from that list can be in D- or L-stereoconfiguration.
X² represents a natural amino acid selected from a list consisting of F, H. Y or y, whereas any amino acid from that list can be in D- or L-stereoconfiguration,
X³ represents the natural amino acid P, or an unnatural amino acid selected from a list consisting of L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid. L-trans-3-hydroxyproline, (2S,4S)-4-fluoroproline ((cis-4-Fluoro)P), L-4,4-difluoroproline ((Difluoro)P), X represents a natural amino acid selected from a list consisting of Q, A and K, whereas any natural amino acid can be in D- or L-stereoconfiguration,
X⁵ represents a natural amino acid selected from a list consisting of F, H. W or Y,
X⁶ represents a natural amino acid selected from a list consisting of Q, A and K, whereas any natural amino acid can be in D- or L-stereoconfiguration,
   wherein the amino group of K might be substituted with 6-Carboxytetramethylrhodamine (Tam),
X⁷ represents a natural amino acid selected from a list consisting of F, H, W or Y,
or a pharmaceutically acceptable salt, hydrate, solvate or solvate of the salt,
   with the proviso, that compound YFP[cQFAFC] is excluded.

Particular preference is given in the context of the present invention to compounds of the formula (I) in which
R¹ is absent
   or
   represents 6-Carboxytetramethylrhodamine (Tam), or the sequence R⁴GFLG ##,
      wherein
      ## marks the attachment to the terminal amino group of X¹.
      R⁴ represents

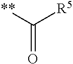

wherein
R⁵ represents methyl,
or
represents a group of the formula (IIIa)

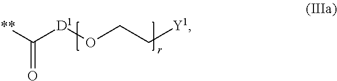

wherein
* marks the attachment to the terminal amino group of X¹,
D¹ is ethylene,
Y¹ is amino.
   wherein amino might be substituted with 6-carboxytetramethylrhodamine (Tam) via an amide bond,
and
r represents 4,
R² represents a group of the formula (II)

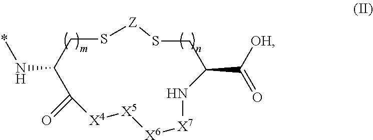

wherein
* represents the attachment to the carbonyl atom of the carboxy group of X³,
Z represents a bond or —CH₂—,
m represents 1 or 2,
n represents 1 or 2,
X¹ represents Y or y.
X² represents F,
X³ represents P,
X⁴ represents Q,
X⁵ represents F,
X⁶ represents A or K,
X⁷ represents F or W,
or a pharmaceutically acceptable salt, hydrate, solvate or solvate of the salt thereof,
   with the proviso, that compound YFP[cQFAFC] is excluded.

According to a further embodiment, the invention provides compounds according to formula (I), wherein
R¹ is absent
   or
   represents 6-Carboxytetramethylrhodamine (Tam) or the sequence R⁴GFLG ##,
      wherein
      ## marks the attachment to the terminal amino group of X.
      R₄ represents

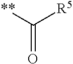

wherein
R⁵ represents methyl,
or
represents a group of the formula (IIIa)

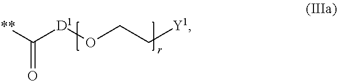

wherein
** marks the attachment to the terminal amino group of X¹,
   D¹ is ethylene,
   Y¹ is amino,
      wherein amino might be substituted with 6-carboxytetramethylrhodamine (Tam) via an amide bond,
   and
   r represents 4.

According to a further embodiment, the invention provides compounds according to formula (I), wherein
R² represents a group of the formula (II)

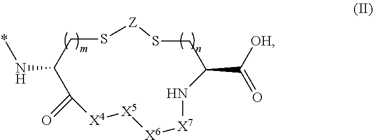

wherein
* represents the attachment to the carbonyl atom of the carboxy group of X³,
Z represents a bond or —CH₂—,
m represents 1 or 2,
n represents 1 or 2.
X¹ represents a natural amino acid selected from a list consisting of F, H, Y or y, whereas any amino acid from that list can be in D- or L-stereoconfiguration,
X² represents a natural amino acid selected from a list consisting of F, H, Y or y, whereas any amino acid from that list can be in D- or L-stereoconfiguration,
X³ represents the natural amino acid P, or an unnatural amino acid selected from a list consisting of L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline, (2S,4S)-4-fluoroproline ((cis-4-Fluoro)P), L-4,4-difluoroproline ((Difluoro)P).
X⁴ represents a natural amino acid selected from a list consisting of Q, A and K, whereas any natural amino acid can be in D- or L-stereoconfiguration,
X⁵ represents a natural amino acid selected from a list consisting of F, H, W or Y,
X⁶ represents a natural amino acid selected from a list consisting of Q. A and K, whereas any natural amino acid can be in D- or L-stereoconfiguration.
   wherein the amino group of K might be substituted with 6-Carboxytetramethylrhodamine (Tam),
X⁷ represents a natural amino acid selected from a list consisting of F, H, W or Y,

According to a further embodiment, the invention provides compounds according to formula (I), wherein
R² represents a group of the formula (II)

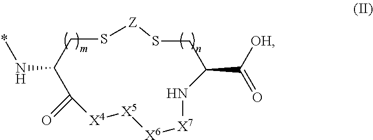

wherein
* represents the attachment to the carbonyl atom of the carboxy group of X³,
Z represents a bond or —CH₂—,
m represents 1 or 2,
n represents 1 or 2,
X¹ represents Y or v.
X² represents F.
X³ represents P,
X⁴ represents Q,
X⁵ represents F,
X⁶ represents A or K,
X⁷ represents F or W.

According to a further embodiment, the invention provides compounds according to formula (I), wherein
X¹ represents a natural amino acid selected from a list consisting of F, H, Y or y, whereas any amino acid from that list can be in D- or L-stereoconfiguration.

According to a further embodiment, the invention provides compounds according to formula (I), wherein
X² represents a natural amino acid selected from a list consisting of F, H, Y or y, whereas any amino acid from that list can be in D- or L-stereoconfiguration.

According to a further embodiment, the invention provides compounds according to formula (I), wherein
X³ represents the natural amino acid P, or an unnatural amino acid selected from a list consisting of L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline, (2S,4S)-4-fluoroproline ((cis-4-Fluoro)P), L-4,4-difluoroproline ((Difluoro)P).

According to a further embodiment, the invention provides compounds according to formula (I), wherein
X⁴ represents a natural amino acid selected from a list consisting of Q, A and K, whereas any natural amino acid can be in D- or L-stereoconfiguration.

According to a further embodiment, the invention provides compounds according to formula (I), wherein
X⁵ represents a natural amino acid selected from a list consisting of F, H, W or Y.

According to a further embodiment, the invention provides compounds according to formula (I), wherein
X⁶ represents a natural amino acid selected from a list consisting of Q, A and K, whereas any natural amino acid can be in D- or L-stereoconfiguration.

According to a further embodiment, the invention provides compounds according to formula (I), wherein
X⁷ represents a natural amino acid selected from a list consisting of F, H, W or Y.

According to a further embodiment, the invention provides compounds according to formula (I), wherein
X¹ represents Y or y.

According to a further embodiment, the invention provides compounds according to formula (I), wherein
X² represents F.

According to a further embodiment, the invention provides compounds according to formula (I), wherein
X³ represents P.

According to a further embodiment, the invention provides compounds according to formula (I), wherein
X⁴ represents Q.

According to a further embodiment, the invention provides compounds according to formula (I), wherein
X⁵ represents Q.

According to a further embodiment, the invention provides compounds according to formula (I), wherein
X⁶ represents A or K.

According to a further embodiment, the invention provides compounds according to formula (I), wherein
X⁷ represents a natural amino acid selected from a list consisting of F or Y.

According to a further embodiment, the invention provides compounds according to formula (I), wherein

compounds YFP[cQFAFC] and yFP[xQFAWC] are excluded.

The peptide of the present invention can comprise a C₈-C₂₀ fatty acid. Generally, such fatty acid may be branched or cyclic. The C₈-C₂₀ fatty acid is preferably bound to the N-terminal. The C₈-C₂₀ fatty acid can be bound to any suitable functional group of a chemical group and/or amino acid of the peptide, e.g. hydroxyl group, carboxyl group, amino group, thiol group, preferably an amino or carboxy group. Preferably the C₈-C₂₀ fatty acid is bound to the N-terminal end via an amide bond. Preferably the fatty acid side chain formed by R¹ is a fatty acid >C₁₀, more preferably a C₁₄-, C₁₆- or C₁₈-fatty acid.

The term “mimetic”, used in context with some amino acids in the definition of several moieties of the peptide according to formula (I) or formula (II) of the present invention, represents a respective amino acid mimetic, such as e.g. an arginine mimetic, an isoleucine mimetic or a proline mimetic. Generally, a “protein mimetic” indicates a molecule such as a peptide, a modified peptide or any other molecule that biologically mimics the action or activity of some other protein. In context with the use of the term “mimetic” in connection with a certain amino acid said term “mimetic” analogously indicates any other amino acid, amino acid analogue, amino acid derivative, amino acid conjugate or the like, which biologically mimics the action or activity of the respective amino acid.

Proline mimetics according to the present invention comprise in particular (1S,2S,5R)-3-Azabicyclo[3.1.0]hexane-2-carboxylic acid, Hyp, Morpholine-3-carboxylic. Pip, (4aR,6aR,9S,11aS)-11-Oxo-2,3,4,4a,6a,7,8,9,11,11a-decahydro-1H-pyrido[3,2-e]pyrrolo[1,2-a]azepine-9-carboxylic acid or (trans-4-Fluoro)P, (1R,2S,5S)-3-azabicyclo[3.1.0]hexane-2-carboxylic acid, Oic, Hyp, (4-CF3)P, (cis-4-Fluoro)P, 3,3-dimethyl-1,3-azasilolidine-5-carboxylic acid, (3S—OH)P, (1R,3S,5R)-2-Azabicyclo[3.1.0]hexane-3-carboxylic acid, (6S)-5-Azaspiro[2.4]heptane-6-carboxylic acid, rel-(1R,3R,5R,6R)-6-(Trifluoromethyl)-2-azabicyclo[3.1.0]hexane-3-carboxylic acid, (2S,3aS,6aS)-Octahydrocyclopenta[b]pyrrole-2-carboxylic acid or difluoroproline, (3R,6R)-1,1-Difluoro-5-azaspiro[2.4]heptane-6-carboxylic acid (enantiomer 1), (3R,6R)-1,1-Difluoro-5-azaspiro[2,4]heptane-6-carboxylic acid (enantiomer 2) and substituted prolines.

Isoleucine mimetics according to the present invention comprise in particular (N-Methyl)-I, allo-Ile, Cba, Nva, Abu, Leu, Cpg, cyclohexyl-Gly, (S)-2-Amino-3-ethyl-pentanoic acid, 3-Chloro-Phg, allo-Ile, Chg, Cyclobutylglycine, allo-Ile, Cbg, (2S,3S)-2-((Amino)methyl)-3-methylpentanoic acid, Phg, 2-[(1S,2S)-1-(Amino)-2-methylbutyl]-1,3-oxazole-4-carboxylic acid, 2-Methyl-D-alloisoleucine, Nva, Abu or Ala.

Leucine mimetics according to the present invention comprise in particular (tBu)A, (2-Chloro)F, (2-Bromo)F, AAD, (2S)-2-Amino-4,4,4-trifluorobutanoic acid, Cnba, (4-Fluoro)L, (S)-(trifluoromethyl)L-cysteine, (2S)-2-amino-3-(1-methylcyclopropyl)propanoic acid, Gly(tBu), 3-(Trimethylsilyl)-L-alanine, 2,5-difluoro-L-phenylalanine, 2-Amino-7-(tert-butoxy)-7-oxoheptanoic acid, 5,5,5-Trifluoro-L-leucine ((Trifluoro)L), (2-Me)F, Cba, Cpa, cyclopropylmethylalanine, trifluoromethylalanine or difluoromethylalanine, (2-Fluoro)F, (2S)-3-(2,3-difluorophenyl)-2-aminopropanoic acid, (2S)-3-(3-cyanophenyl)-2-aminopropanoic acid, 2-Amino-5,5,5-trifluoro-4-methyl-pentanoic acid, (2S)-2-Amino-5-methyl-hexanoic acid or (2S)-3-(indol-4-yl)-2-(amino)propanoic acid.

The invention further comprises analogues and derivatives of the described peptides. The term “analogue” or “derivative” of a peptide or an amino acid sequence according to the present invention comprises in particular any amino acid sequence having a sequence identity of at least 80% or at least 85%, preferably at least 90%, more preferably at least 95%, and even more preferably of at least 99% identity to said sequence, and same or comparable properties or activity. Sequence identity can be determined by common techniques, such as visual comparison or by means of any computer tool generally used in the field. Examples comprise BLAST programs used with default parameters.

An analogue or derivative of a peptide or an amino acid sequence of the invention may result from changes derived from mutation or variation in the sequences of peptides of the invention, including the deletion or insertion of one or more amino acids or the substitution of one or more amino acids, or even to alternative splicing. Several of these modifications may be combined. Preferably, an analogue of an amino acid sequence of the invention comprises conservative substitutions relative to the sequence of amino acids.

The term “conservative substitution” as used herein denotes that one or more amino acids are replaced by another, biologically similar residue. Examples include substitution of amino acid residues with similar characteristics, e.g., small amino acids, acidic amino acids, polar amino acids, basic amino acids, hydrophobic amino acids and aromatic amino acids. See, for example, the scheme in Table 4 below, wherein conservative substitutions of amino acids are grouped by physicochemical properties. I: neutral, hydrophilic; II: acids and amides; III: basic; IV: hydrophobic; V: aromatic, bulky amino acids, VI: neutral or hydrophobic; VII: acidic; VIII: polar.

TABLE 4
Amino Acids grouped according to
their physicochemical properties

| I | II | III | IV | V | VI | VII | VIII |
|---|---|---|---|---|---|---|---|
| Ala | Asn | His | Met | Phe | Ala | Glu | Met |
| Ser | Asp | Arg | Leu | Tyr | Leu | Asp | Ser |
| Thr | Glu | Lys | Ile | Trp | Ile | | Thr |
| Pro | Gln | | Val | | Pro | | Cys |
| Gly | | | Cys | | Gly | | Asn |
| | | | | | Val | | Gln |

All peptides of this invention unless otherwise noted are TFA salts. The invention comprises further pharmaceutically acceptable salts of the peptides as defined herein and salt free forms. Therein, pharmaceutically acceptable salts represent salts or zwitterionic forms of the peptides or compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, carbonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, sulfate, tartrate, trichloroacetate, trifluoroacetate, phosphate, glutamate, bicarbonate, para-toluenesulfonate, and undecanoate. Preferred acid addition salts include trifluoroacetate, formate, hydrochloride, and acetate.

The peptide of the present invention can be substituted with a suitable water soluble polymer characterized by repeating units. Suitable polymers may be selected from the group consisting of polyalkyloxy polymers, hyaluronic acid and derivatives thereof, polyvinyl alcohols, polyoxazolines, polyanhydrides, poly(ortho esters), polycarbonates, polyurethanes, polyacrylic acids, polyacrylamides, polyacrylates, polymethacrylates, polyorganophosphazenes, polysiloxanes, polyvinylpyrrolidone, polycyanoacrylates, and polyesters.

The peptides of the present invention can be substituted with at least one polyethylene group (PEG group). The PEG group is preferably bound to the N-terminal end. The PEG group can be bound to any suitable functional group of a chemical group and/or amino acid of the peptide, e.g. hydroxyl group, carboxyl group, amino group, thiol group, preferably an amino or carboxy group. Preferably the peptide according to the invention contains one PEG group bound to the N-terminal end. More preferably the one PEG group is bound to the N-terminal via an amide bond.

A PEG group according to the invention is any group containing at least two ethylene oxide units to form an oligomer or polymer ethylene oxide.

Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen. e.g., among acid addition salts and basic salts. Examples of acid addition salts include chloride salts, citrate salts and acetate salts.

Examples of basic salts include salts where the cation is selected from alkali metal cations, such as sodium or potassium ions, alkaline earth metal cations, such as calcium or magnesium ions, as well as substituted ammonium ions, such as ions of the type N(R₁)(R²)(R³)(R⁴), where R¹, R², R³ and R¹ independently from each other will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Therein, salts where the cation is selected among sodium, potassium and calcium are preferred.

Other examples of pharmaceutically acceptable salts are described in “Remington's Pharmaceutical Sciences”, 17th edition. Alfonso R Gennaro (Ed.), Mark Publishing Company, Easton. PA, USA. 1985 (and more recent editions thereof), in the “Encyclopedia of Pharmaceutical Technology” 3rd edition, James Swarbrick (Ed). Informa Healthcare USA (Inc.). NY, USA. 2007. Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts, preferably choline. Hemi-salts of acids and bases may also be formed, e.g., hemisulphate and hemicalcium salts.

The invention further comprises solvates of the peptides as defined herein. Therein the term “solvate” refers to a complex of defined stoichiometry formed between a solute (e.g., a peptide according to the invention or pharmaceutically acceptable salt thereof) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The compounds according to the invention show an unforeseeable useful spectrum of pharmacological activity.

Accordingly they are suitable for use as medicaments for treatment and/or prevention of diseases in humans and animals.

On the basis of their pharmacological properties, the compounds according to the invention can be employed for treatment and/or prevention of cardiovascular diseases, metabolic disorders, in particular diabetes mellitus and its consecutive symptoms, such as e.g. diabetic macro- and microangiopathy, diabetic nephropathy and neuropathy.

The compounds are moreover suitable for treatment and/or prevention of obesity.

The compounds are moreover suitable for treatment and/or prevention of asthmatic diseases.

The compounds according to the invention are furthermore suitable for treatment and/or prevention of inflammatory disorders of the gastrointestinal tract such as inflammatory bowel disease. Crohn's disease, ulcerative colitis, and toxic and vascular disorders of the intestine and for the treatment and/or prevention of sepsis (SIRS), multiple organ failure (MODS, MOF), inflammatory disorders of the kidney, chronic intestinal inflammations (IBD, Crohn's disease, ulcerative colitis), pancreatitis, peritonitis, cystitis, urethritis, prostatitis, epidimytitis, oophoritis, salpingitis, vulvovaginitis, rheumatoid disorders, osteoarthritis, inflammatory disorders of the central nervous system, multiple sclerosis, inflammatory skin disorders and inflammatory eye disorders.

Moreover, the compounds of the invention are suitable for treatment of cancers, for example skin cancer, brain tumours, breast cancer, bone marrow tumours, leukaemias, liposarcomas, carcinomas of the gastrointestinal tract, of the liver, the pancreas, the lung, the kidney, the ureter, the prostate and the genital tract and also of malignant tumours of the 10 lymphoproliferative system, for example Hodgkin's and non-Hodgkin's lymphoma.

Further disclosed is a method for the treatment and/or prophylaxis of metabolic disorders, diabetes mellitus, obesity, asthmatic diseases, inflammatory disorders and cancer in humans or animals using an effective amount of at least one a compound of formula (I), a physiologically acceptable salt, a solvate or a solvate of a salt according to the invention or to one of the embodiments disclosed herein or a medicament comprising a compound of formula (I), a physiologically acceptable salt, a solvate or a solvate of a salt according to the invention or to one of the embodiments disclosed herein.

The invention further provides a process for preparing the compounds of the formula (I), or salts thereof, solvates thereof or the solvates of salts thereof.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The compounds of formula (I), a physiologically acceptable salt, a solvate or a solvate of a salt according to the invention can be used in a method for the treatment and/or prevention of metabolic disorders, cancer and/or inflammatory disorders.

In accordance with a further aspect, the present invention thus further provides for the use of the compounds according to the invention for treatment and/or prevention of disorders, especially of the aforementioned disorders.

In accordance with a further aspect, the present invention further provides for the use of the compounds according to the invention for production of a medicament for treatment and/or prevention of disorders, especially of the aforementioned disorders.

In accordance with a further aspect, the present invention further provides a medicament comprising at least one of the compounds according to the invention for treatment and/or prevention of disorders, especially of the aforementioned disorders.

In accordance with a further aspect, the present invention further provides for the use of the compounds according to the invention in a method for treatment and/or prevention of disorders, especially of the aforementioned disorders.

In accordance with a further aspect, the present invention further provides a method of treatment and/or prevention of disorders, especially of the aforementioned disorders, using an effective amount of at least one of the compounds according to the invention.

In accordance with a further aspect, the compounds of general formula (I), as described supra, or stereoisomers, tautomers, hydrates, solvates, and salts thereof, particularly pharmaceutically acceptable salts thereof, or mixtures of same, are suitable for the treatment and/or prophylaxis of diabetes mellitus, obesity, asthmatic diseases, inflammatory disorders and cancer.

In accordance with a further aspect, the present invention thus further provides for the use of the compounds according to the invention for treatment and/or prevention of diabetes mellitus, obesity, asthmatic diseases, inflammatory disorders and cancer.

In accordance with a further aspect, the present invention further provides for the use of the compounds according to the invention for production of a medicament for treatment and/or prevention of diabetes mellitus, obesity, asthmatic diseases, inflammatory disorders and cancer.

In accordance with a further aspect, the present invention further provides a medicament comprising at least one of the compounds according to the invention for treatment and/or prevention of diabetes mellitus, obesity, asthmatic diseases, inflammatory disorders and cancer.

In accordance with a further aspect, the present invention further provides for the use of the compounds according to the invention in a method for treatment and/or prevention of diabetes mellitus, obesity, asthmatic diseases, inflammatory disorders and cancer.

In accordance with a further aspect, the present invention further provides a method of treatment and/or prevention of disorders, especially of diabetes mellitus, obesity, asthmatic diseases, inflammatory disorders and cancer, using an effective amount of at least one of the compounds according to the invention.

It is possible for the cyclic chemerin-9 peptide of the present invention to act systemically and/or locally. For this purpose, they can be administered in a suitable way, for example by the parenteral, pulmonary, nasal, sublingual, lingual, buccal, dermal, transdermal, conjunctival, optic route or as implant or stent.

The compounds according to the invention can be administered in administration forms suitable for these administration routes.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration include preparations for injection and infusion in the form of solutions, suspensions, emulsions, lyophilizates or sterile powders.

Suitable for the other administration routes are, for example, pharmaceutical forms for inhalation (including powder inhalers, nebulizers), nasal drops, eye drops, solutions or sprays; films/wafers or aqueous suspensions (lotions, shaking mixtures), lipophilic suspensions, ointments, creams, transdermal therapeutic systems (e.g. patches), milk, pastes, foams, dusting powders, implants or stents.

Parenteral administration is preferred, especially intravenous administration. Inhalative administration is also preferred, e.g. by using powder inhalers or nebulizers.

The compounds according to the invention can be converted into the stated administration forms. This can take place in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colors (e.g. inorganic pigments, for example iron oxides) and masking flavors and/or odors.

It has generally been found to be advantageous, in the case of parenteral administration, to administer amounts of about 0.001 to 5 mg/kg, preferably about 0.01 to 1 mg/kg, of body weight to achieve effective results.

It may nevertheless be necessary in some cases to deviate from the stated amounts; in particular as a function of the body weight, route of administration, individual response to the active ingredient, nature of the preparation and time or interval over which administration takes place. For instance, less than the aforementioned minimum amount may be sufficient in some cases, whereas in other cases the stated upper limit must be exceeded. In the case of administration of larger amounts, it may be advisable to divide these into a plurality of individual doses over the day.

According to a further embodiment, the invention provides a pharmaceutical composition comprising at least one compound containing a peptide which may be isolated and/or purified, comprising, essentially consisting of, or consisting of, formula (I) or formula (II) or a derivative, prodrug, analogue, pharmaceutically acceptable salt, solvate or solvate of the salt, in combination with one or more inert, non-toxic, pharmaceutically suitable excipients.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscannellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition comprising at least one peptide, derivative or analogue as defined herein or a pharmaceutically acceptable salt or solvate thereof or a complex as defined above.

In particular, the present invention relates to a pharmaceutical composition comprising at least one peptide, derivative or analogue as defined herein or a pharmaceutically acceptable salt or solvate thereof or a complex as defined above, conventionally together with one or more pharmaceutically suitable excipient(s), and to their use according to the present invention.

A pharmaceutical composition according to the present invention may comprise at least one additional active ingredient, such as preferably an additional active ingredient which is active in the prophylaxis and/or treatment of the disorders or diseases as defined herein.

The at least one peptide, derivative or analogue as defined herein or the pharmaceutically acceptable salt or solvate thereof or the complex or the pharmaceutical compositions as defined above may be administered enterally or parenterally, including intravenous, intramuscular, intraperitoneal, intrasternal, subcutaneous, intradermal and intraarticular injection and infusion, orally, intravaginally, intraperitoneally, intrarectally, topically or buccally. Suitable formulations for the respective administration routes are well known to a skilled person and include, without being limited thereto: pills, tablets, enteric-coated tablets, film tablets, layer tablets, sustained-release or extended-release formulations for oral administration, plasters, topical extended-release formulations, dragees, pessaries, gels, ointments, syrup, granules, suppositories, emulsions, dispersions, microcapsules, microformulations, nanoformulations, liposomal formulations, capsules, enteric-coated capsules, powders, inhalation powders, microcrystalline formulations, inhalation sprays, powders, drops, nose drops, nasal sprays, aerosols, ampoules, solutions, juices, suspensions, infusion solutions or injection solutions, etc.

The suitable dosage of the cyclic chemerin-9 peptide of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient: d) the time of administration, route of administration, and rate of excretion of the specific hepcidin analogue employed; e) the duration of the treatment: f) drugs used in combination or coincidental with cyclic chemerin-9 derivative according to the invention employed, and like factors well known in the medical arts.

In particular embodiments, the total daily dose of the cyclic chemerin-9 derivative of the invention to be administered to a subject or patient in single or divided doses may be in amounts, for example, from 0.0001 to 300 mg/kg body weight daily or 1 to 300 mg/kg body weight daily, or from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, administered in one or more doses, such as from one to three doses. Generally, the cyclic chemerin-9 derivative of the invention may be administered continuously (e.g. by intravenous administration or another continuous drug administration method), or may be administered to a subject at intervals, typically at regular time intervals, depending on the desired dosage and the pharmaceutical composition selected by the skilled practitioner for the particular subject. Regular administration dosing intervals include, e.g., once daily, twice daily, once every two, three, four, five or six days, once or twice weekly, once or twice monthly, and the like.

The invention further comprises the use of the cyclic chemerin-9 derivative as described herein for the manufacture of a medicament, in particular for the manufacture of a medicament for the prophylaxis and/or treatment of a disorder or disease as defined herein.

The invention further comprises a process for manufacturing the peptides of the present invention, derivative or analogue or the pharmaceutically acceptable salt or solvate thereof or a complex, each as described herein. The process for manufacturing comprises the steps as shown in the examples of the present invention.

Generally, the cyclic chemerin-9 derivative of the present invention may be manufactured synthetically, or semi-recombinantly.

According to a further embodiment, the invention provides a process for preparing a compound containing a peptide which may be isolated and/or purified, comprising, essentially consisting of, or consisting of, formula (I) or formula (II) or a derivative, prodrug, analogue or pharmaceutically acceptable salts or solvates thereof by using solid phase peptide synthesis.

According to a further embodiment, the invention provides a process for preparing a compound containing a peptide which may be isolated and/or purified, comprising, essentially consisting of, or consisting of, formula (I) or formula (II) or a derivative, prodrug, analogue, pharmaceutically acceptable salt, solvate or solvate of the salt, containing the steps
1. Use of a 2-chlorotrityl-type resin with a loading of 0.2-1.0 mmol/g, or a Wang-type resin with a loading of 0.2-1.0 mmol/gram,
2. Loading the c-terminal amino acid of the sequence onto the resin,
3. Removal of fmoc protection with a 15-25% piperidine solution in DMF or NMP,
4. Coupling of the next amino acid in the sequence with coupling reagents such as HBTU, HATU or DIC/Oxyma using stoichiometries between 3-8 equivalents,
5. Repeating steps 3 and 4 until the sequence is completed.
6. Cleavage of the peptide from the solid support using a cleavage cocktail that involves TFA and a thiol scavenger,
7. Cyclization of two cysteines in the sequence under oxidative conditions (air or I2),
8. Purification of the cleaved peptide using reversed-phase HPLC.
   or
1. Use of a 2-chlorotrityl-type resin with a loading of 0.2-1.0 mmol/g, or a Wang-type resin with a loading of 0.2-1.0 mmol/gram,
2. Loading the c-terminal amino acid of the sequence onto the resin,
3. Removal of fmoc protection with a 15-25% piperidine solution in DMF or NMP,
4. Coupling of the next amino acid in the sequence with coupling reagents such as HBTU, HATU or DIC/Oxyma using stoichiometries between 3-8 equivalents,
5. Repeating steps 3 and 4 until the sequence is completed,
6. Cleavage of the peptide from the solid support using a cleavage cocktail that involves TFA and a thiol scavenger,
7. Either a) Cyclization of two cysteines in the sequence under oxidative conditions (air or I₂), or b) Purification of the cleaved peptide using reversed-phase HPLC, followed by cyclization by reaction with CH₂I₂.
8. Purification of the cyclic peptide using reversed-phase HPLC.

The invention is further illustrated by the following examples, which relate to certain specific embodiments of the present invention. The examples were carried out using well known standard techniques within the routine to those of skill in the art, unless indicated otherwise. The following examples are for illustrative purposes only and do not purport to be wholly definitive as to conditions or scope of the invention. As such, they should not be construed in any way as limiting the scope of the present invention.

The percentages in the following tests and examples are, unless stated otherwise, percentages by weight; parts are parts by weight. Solvent ratios, dilution ratios and concentration data for the liquid/liquid solutions are each based on volume.

### Experimental Section—Synthesis

### Abbreviations

ACN: acetonitrile, BRET: Bioluminescence resonance energy transfer, CCRL2: Chemokine (C-C)-motif receptor-like 2, CMKLR1: chemokine-like receptor 1, DCM: dichloromethane, Dde: N-[1-(4,4-dimethyl-2,6-dioxocyclohex-1-ylidene)ethyl], DIC: N′,N′-diisopropyl carbodiimide, DIPEA: N,N-diisopropylethylamine, DMEM: Dulbecco's Modified Eagle's Medium, DMF: dimethylformamide, EDTA: ethylenediaminetetraacetic acid, EG(4): polyethylene glycol consisting of 4 ethylenoxide groups, ESI-MS: electrospray ionization mass spectrometry, Et₂O: diethyl ether, equiv: equivalents, FBS: fetal bovine serum, Fmoc: 9-fluorenylmethoxycarbonyl, GPCR: G protein-coupled receptor, GPR1: G protein-coupled receptor 1. HATU: O-(7-Azabenzotriazol-1-yl)-N,N,N,N-tetramethyluronium hexafluorophosphate, HBSS: Hank's buffered saline solution, HEK: human embryonic kidney, HOBt: hydroxy benzotriazole, oxyma: 2-cyano-2-(hydroxyimino) acetic acid ethyl ester. MALDI-ToF: matrix assisted laser desorption/ionization—time of flight (MS), PBS: phosphate-buffered-saline, PEG: polyethylene glycol; RP-HPLC: reversed phase high pressure liquid chromatography, rt: room temperature, TA: thioacetal, Tam: 6-carboxytetramethylrhodamine, tBu: tert-butyl, TCEP: tris(2-carboxyethyl)phosphine hydrochloride, TFA: trifluoracetic acid. THF: tetrahydrofuran. X: homocysteine, YFP: yellow fluorescent protein.

### Nomenclature of Amino Acids and Peptide Sequences is According to:

International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624

| Trivial Name | Symbol | One-letter symbol |
|---|---|---|
| Alanine | Ala | A |
| Glutamine | Gln | Q |
| Glycine | Gly | G |
| Phenylalanine | Phe | F |
| Proline | Pro | P |
| Serine | Ser | S |
| Homocysteine | Hcys | X |
| Tyrosine | Tyr | Y |

### Nomenclature of Non-Proteinogenic Amino Acids:

| Trivial Name | Symbol | One-letter symbol |
|---|---|---|
| D-Cysteine | D-Cys | c |
| D-Homocysteine | D-Hcys | x |
| D-Tyrosine | D-Tyr | y |

### Materials

### Peptide Synthesis:

Fmoc-protected amino acids were purchased from ORPEGEN (Heidelberg, Germany). Peptide resins, 1-hydroxybenztotriazole (HOBt), diiodomethane, ethanedithiol (EDT), diethyl ether and trifluoracetic acid (TFA), were obtained from Merck (Darmstadt, Germany). N,N′-diisopropylcarbodiimide (DIC) and 2-cyano-2-(hydroxyimino) acetic acid ethyl ester (oxyma) were purchased from Iris Biotech (Marktredwitz, Germany). Dimethylformamide (DMF) and dichloromethane (DCM) were purchased from Biosolve (Valkenswaard, Netherlands), acetonitrile (ACN) was obtained from VWR (Darmstadt, Germany), and tetrahydrofuran (THF) was purchased from Grassing (Filsum, Germany). O-(7-Azabenzotriazol-1-yl)-N,N,N,N-tetramethyluronium hexafluorophosphate (HATU), tris(2-carboxyethyl)phosphine hydrochloride (TCEP), N,N-diisopropylethylamine (DIPEA), piperidine and thioanisole were obtained from Sigma-Aldrich (St. Louis, USA). 6-Carboxytetramethylrhodamine (Tam) was purchased from emp biotech (Berlin, Germany). Triethylamine (Et₃N) was purchased from Thermo Fisher Scientific (Waltham, USA).

### Cell Culture:

Cell culture media (Dulbecco's Modified Eagle's Medium (DMEM), Ham's F12), as well as trypsin-EDTA. Dulbecco's Phosphate-Buffered Saline (DPBS), and Hank's Balanced Salt Solution (HBSS) were obtained from Lonza (Basel, Switzerland). Fetal bovine serum (FBS) was from Biochrom GmbH (Berlin, Germany). Hygromycin B was purchased from Invivogen (Toulouse, France) and Opti-MEM was obtained from Life Technologies (Basel, Switzerland). Lipofectamine™ 2000 was obtained from Invitrogen (Carlsbad, CA, USA). MetafectenePro™ was received from Biontex Laboratories GmbH (Manchen, Germany). Coelenterazine H was purchased DiscoverX (Fremont, CA, USA), Hoechst33342 nuclear stain was obtained from Sigma-Aldrich (St. Louis, MO. USA). Bovine arrestin-3 was fused to mCherry for fluorescence microscopy or to Rluc8 and cloned into pcDNA3 vector for BRET studies. Pluronic and Fluo-2 AM were obtained from Abcam (Cambridge, UK), Probenicid was purchased from Sigma-Aldrich (St. Louis, USA). The sequence for the chimeric G protein GαΔ6qi4myr was kindly provided by E. Kostenis, Rheinische Friedrich-Wilhelms-Universitat, Bonn, Germany.

### General Methods for Peptide Synthesis

All peptides were synthesized using an orthogonal 9-fluorenylmethoxycarbonyl/tert-butyl (Fmoc/tBu) solid-phase peptide synthesis strategy. Standard synthesis of all peptides was performed on a Syro II peptide synthesizer (MultiSynTech, Bochum. Germany) on a scale of 15 μmol. Peptides were synthesized on a Wang resin preloaded with the first amino acid unless stated otherwise. Coupling reactions during automated peptide synthesis were performed twice with 8 equiv of the respective, Fmoc-protected amino acid activated in situ with equimolar amounts of oxyma and DIC in DMF for 30 min. Fmoc-deprotection was achieved by incubation with 40% piperidine in DMF (v/v) for 3 min and 20% piperidine in DMF (v/v) for 10 min. All reactions were performed at room temperature unless stated otherwise. All peptides were purified by preparative RP-HPLC on a Kinetex 5 μm XB-C₁₈ 100 Å or a Jupiter 4 μm Proteo 90 Å C12 column (Phenomenex, Torrence, USA). Purity was confirmed by RP-HPLC on a Jupiter 4 μm Proteo 90 Å C₁₂ (Phenomenex), a Kinetex 5 μm biphenyl 100 Å (Phenomenex) or an Aeris 3.6 μm 100 Å XB-C18 (Phenomenex) column. RP-HPLC was performed employing linear gradients of eluent B (0.08% TFA in ACN) in eluent A (0.1% TFA in H₂O). MALDI-ToF MS on an Ultraflex II and ESI MS on an HCT ESI (Bruker Daltonics, Billerica, USA) were utilized to verify product identity.

### SPECIFIC EXAMPLES

### Compound List:

| Nr | Compound name | Sequence |
|---|---|---|
| ($$ 1) | Chemerin-9 | YFPGQFAFS |
| ($$ 2) | Tam-chemerin-9 | Tam-YFPGQFAFS |
| ($$ 3) | Tam-EG4-chemerin-9 | Tam-EG(4)-YFPGQFAFS |
| ($$ 4) | [c4,C9]-chemerin-9 | YFP[cQFAFC] |
| ($$ 5) | Tam-[c4,C9]-chemerin-9 | Tam-YFP[cQFAFC] |
| ($$ 6) | [c4,C7]-chemerin-9 | YFP[cQFC]FS |
| ($$ 7) | [c4,C9-TA]-chemerin-9 | YFP[c(CH₂)QFAFC] |
| ($$ 8) | Tam-[c4,C9-TA]-chemerin-9 | Tam-YFP[c(CH₂)QFAFC] |
| ($$ 9) | [c4,X9-TA]-chemerin-9 | YFP[c(CH₂)QFAFX] |
| ($$ 10) | Tam-[c4,X9-TA]-chemerin-9 | Tam-YFP[c(CH₂)QFAFX] |
| ($$ 11) | [y1,c4,X9-TA]-chemerin-9 | yFP[c(CH₂)QFAFX] |
| ($$ 12) | [y1,c4,K7(Tam),C9]-chemerin-9 | yFP[cQFK(Tam)FC] |
| ($$ 13) | [x4,C9]-chemerin-9 | YFP[xQFAFC] |
| ($$ 14) | [x4,W8,C9]-chemerin-9 | YFP[xQFAWC] |
| ($$ 15) | [y1,x4,W8,C9]-chemerin-9 | yFP[xQFAWC] |
| ($$ 16) | EG4-[x4,C9]-chemerin-9 | EG(4)-YFP[xQFAFC] |
| ($$ 17) | Tam-EG4-[x4,C9]-chemerin-9 | Tam-EG(4)-YFP[xQFAFC] |
| ($$ 18) | EG4-[x4,W8,C9]-chemerin-9 | EG(4)-YFP[xQFAWC] |
| ($$ 19) | Tam-EG4-[x4,W8,C9]-chemerin-9 | Tam-EG(4)-YFP[xQFAWC] |
| ($$ 20) | GFLG-[x4,C9]-chemerin-9 | GFLGYFP[xQFAFC] |

X = Homocysteine, x = D-Homocysteine, TA = thioacetal.

### Synthesis of Comparison Examples:

### Comparison Example 1: Chemerin-9 ($$ 1)

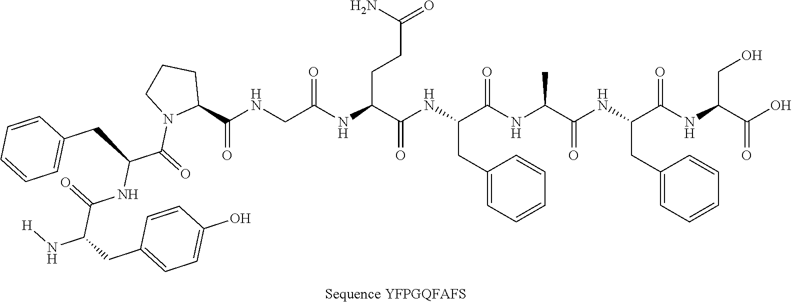 [SMILES: C[C@H](NC(=O)[C@H](Cc1ccccc1)NC(=O)[C@H](CCC(N)=O)NC(=O)CNC(=O)[C@@H]1CCCN1C(=O)[C@H](Cc1ccccc1)NC(=O)[C@@H](N)Cc1ccc(O)cc1)C(=O)N[C@@H](Cc1ccccc1)C(=O)N[C@@H](CO)C(=O)O]

After automated synthesis of YFPGQFAFS the peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Jupiter 4 μm Proteo 90 Å C12 column employing a gradient of 30-60% B in A over 30 min with a flow rate of 15 mL/min, detecting the peptide by measuring the absorption at λ=220 nm. The pure yield was 5.8 mg (36% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with a flow rate of 1.0 and 1.55 mL/min, respectively. The peptide showed over 95% purity as determined by the absorption at 220 nm on both columns, with retention times of 17.4 min and 8.2 min, respectively. The chemical formula of the peptide is C₅₄H₆₆N₁₀O₃ (monoisotopic mass: 1062.5 Da, average mass: 1063.18 Da). The observed masses were in correspondence to the calculated masses, confirming product identity: In MALDI-ToF, signals at m/z=1063.6 [M+H]⁺, m/z=1085.6 [M+Na]⁺ and m/z=1101.5 [M+K]⁺ were observed. In ESI ion trap, signals at m/z=1063.3 [M+H]⁺ and m/z=532.3 [M+2H]²⁺ were observed.

### Comparison Example 2: Tam-chemerin-9 ($$ 2)

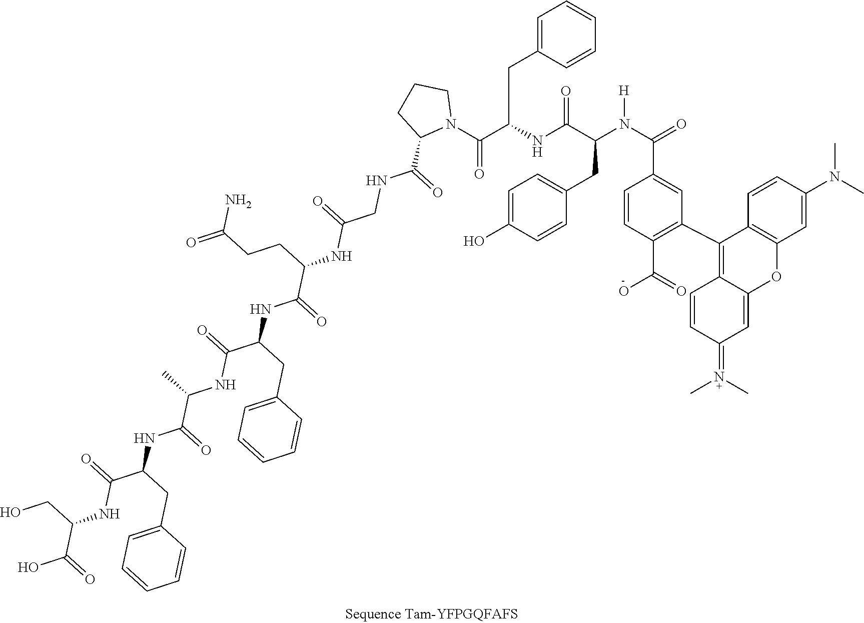

After automated synthesis of YFPGQFAFS, the N-terminus of the peptide was modified with 6-carboxytetramethylrhodamine (Tam) by reaction with 2 equiv of Tam. HATU and DIPEA in DMF overnight, the peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Jupiter 4 μm Proteo 90 Å C12 column employing a gradient of 30-80% B in A over 40 min with a flow rate of 15 mL/min, detecting the peptide by measuring the absorption at λ=220 nm. The pure yield was 5.3 mg (24% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 24.4 min and 19.1 min, respectively. The chemical formula of the peptide is C₇₉H₅₆N₁₂O₁₇ (monoisotopic mass: 1474.6 Da, average mass: 1475.6 Da). The observed masses were in correspondence to the calculated masses, confirming product identity: In MALDI-ToF, signals at m/z=1475.6 [M+H]⁺, m/z=1497.6 [M+Na]⁺ and m/z=1513.5 [M+K]⁺ were observed. In ESI ion trap, signals at m/z=1475.4 [M+H]⁺ and m/z=738.3 [M+2H]²⁺ were observed.

### Comparison Example 3: Tam-EG4-chemerin-9 ($$3)

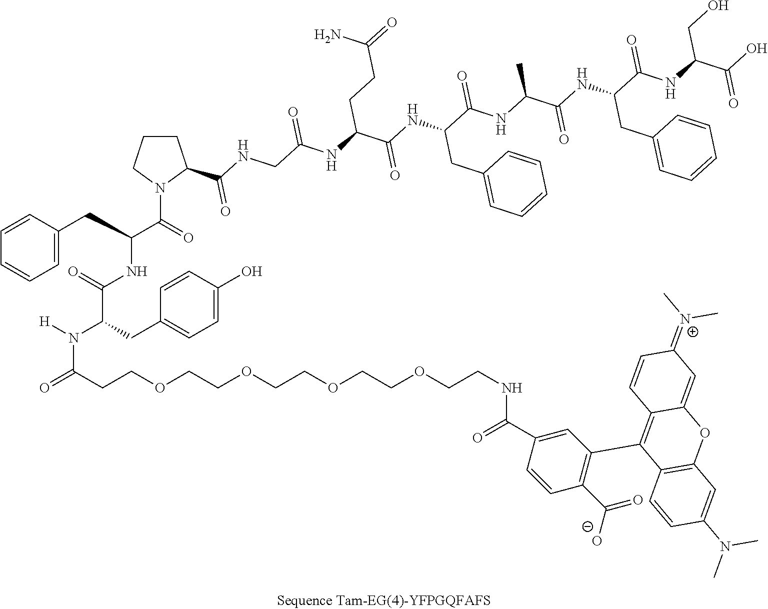 [SMILES: C[C@H](NC(=O)[C@H](Cc1ccccc1)NC(=O)[C@H](CCC(N)=O)NC(=O)CNC(=O)[C@@H]1CCCN1C(=O)[C@H](Cc1ccccc1)NC(=O)[C@H](Cc1ccc(O)cc1)NC(=O)CCOCCOCCOCCOCCNC(=O)c1ccc(C(=O)[O-])c(-c2c3ccc(=[N+](C)C)cc-3oc3cc(N(C)C)ccc23)c1)C(=O)N[C@@H](Cc1ccccc1)C(=O)N[C@@H](CO)C(=O)O]

After automated synthesis of YFPGQFAFS, EG(4) was coupled to the N-terminus of the peptide by reaction of 5 equiv of Fmoc-15-amino-4,7,10,13-tetraoxapentadecanoic acid, HOBt and DIC in DMF overnight. The Fmoc-group was cleaved by reaction with 20% piperidine in DMF for 10 min, the reaction was repeated twice. The peptide was N-terminally modified with 6-carboxytetramethylrhodamine (Tam) by reaction with 2 equiv of Tam. HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Jupiter 4 μm Proteo 90 Å C12 column employing a gradient of 30-80% B in A over 40 min with a flow rate of 15 mL/min, detecting the peptide by measuring the absorption at λ=220 nm. The pure yield was 4.8 mg (18.6% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 24.8 min and 16.4 min, respectively. The chemical formula of the peptide is C₉₀H₁₀₇N₃O₂₂ (monoisotopic mass: 1721.7 Da, average mass: 1722.9 Da). The observed masses were in correspondence to the calculated masses, confirming product identity: In MALDI-ToF, signals at m/z=1722.7 [M+H]⁺, m/z=1745.7 [M+Na]⁺ and m/z=1760.7 [M+K]⁺ were observed. In ESI ion trap, signals at m/z=1722.6 [M+H]⁺ and m/z=862.1 [M+2H]²⁺ were observed.

### Comparison Example 4: [c4,C9]-chemerin-9 ($$ 4)

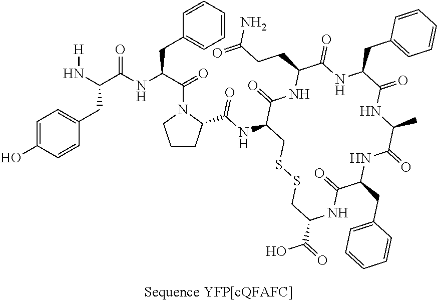 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CSSC[C@@H](C(=O)O)NC(=O)[C@H](Cc2ccccc2)NC1=O]

YFPcQFAFC, were synthesized automatically. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Kinetex 5 μm XB-C18 100 Å column employing a gradient of 25 to 55% B in A over 30 min with a flow rate of 15 mL/min, detecting the peptide by measuring the absorption at λ=220 nm. The pure yield was 1.9 mg (11.1% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with a flow rate of 1.0 and 1.55 mL/min, respectively. The peptide showed over 95% purity as determined by the absorption at 220 nm on both columns, with retention times of 21.2 min and 11.4 min, respectively. The chemical formula of the peptide is C₅₅H₆₆N₁₀O₁₂S₂ (monoisotopic mass: 1122.4 Da, average mass: 1123.3 Da). The observed masses were in correspondence to the calculated masses, confirming product identity. In ESI ion trap, signals at m/z=1123.2 [M+H]⁺ and m/z=562.3 [M+2H]² were observed.

### Comparison Example 5: [c4,C7]-chemerin-9 ($$6)

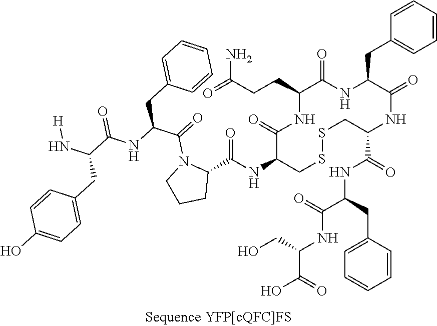 [SMILES: NC(=O)CC[C@@H]1NC(=O)[C@H](NC(=O)[C@@H]2CCCN2C(=O)[C@H](Cc2ccccc2)NC(=O)[C@@H](N)Cc2ccc(O)cc2)CSSC[C@@H](C(=O)N[C@@H](Cc2ccccc2)C(=O)N[C@@H](CO)C(=O)O)NC(=O)[C@H](Cc2ccccc2)NC1=O]

YFPcQFCFS was synthesized by automated peptide synthesis. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Kinetex 5 μm XB-C18 100 Å column employing a gradient of 25 to 55% B in A over 30 min with a flow rate of 15 mL/min, detecting the peptide by measuring the absorption at λ=220 nm. The pure yield was 1.9 mg (11.1% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with a flow rate of 1.0 and 1.55 mL/min, respectively. The peptide showed over 95% purity as determined by the absorption at 220 nm on both columns, with retention times of 21.0 min and 11.7 min, respectively. The chemical formula of the peptide is C₅₅H₆₈N₁₀O₁₂S₂ (monoisotopic mass: 1138.4 Da, average mass: 1139.3 Da). The observed masses were in correspondence to the calculated masses, confirming product identity. In ESI ion trap, signals at m/z=1139.2 [M+H]⁺ and m/z=570.3 [M+2H]²⁺ were observed.

### Synthesis of Examples

### Example 1: Tam-[c4,C9]-chemerin-9 ($$ 5)

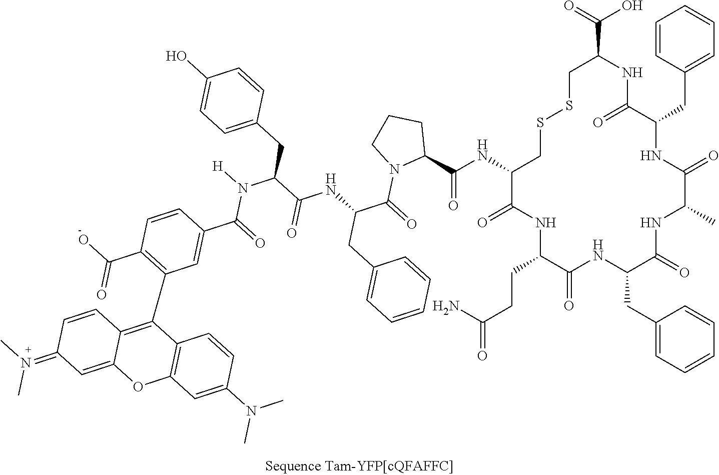

After automated synthesis of YFPcQFAFC, the N-terminus of the peptide was modified with 6-carboxytetramethylrhodamine (Tam) by reaction with 2 equiv of Tam, HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was incubated in TBS, 20% ACN, pH 7.8 for 48 h to promote the formation of the intramolecular disulfide bond. The peptide was purified by RP-HPLC on a Kinetex 5 μm XB-C18 100 Å column employing a gradient of 30 to 65% B in a over 40 min with a flow rate of 15 mL/min. detecting the peptide by measuring the absorption at λ=220 nm. The pure yield was 0.7 mg (3% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 27.5 min and 22.3 min, respectively. The chemical formula of the peptide is C₈₀H₈₆N₁₂O₁₆S₂ (monoisotopic mass: 1534.6 Da, average mass: 1535.8 Da). The observed masses were in correspondence to the calculated masses, confirming product identity: In MALDI-ToF, a signal at m/z=1535.6 [M+H]⁺ was observed. In ESI ion trap, signals at m/z=1535.3 [M+H]⁺ and m/z=768.7 [M+2H]²⁺ were observed.

### Example 2: [c4,C9-TA]-chemerin-9 ($$ 7)

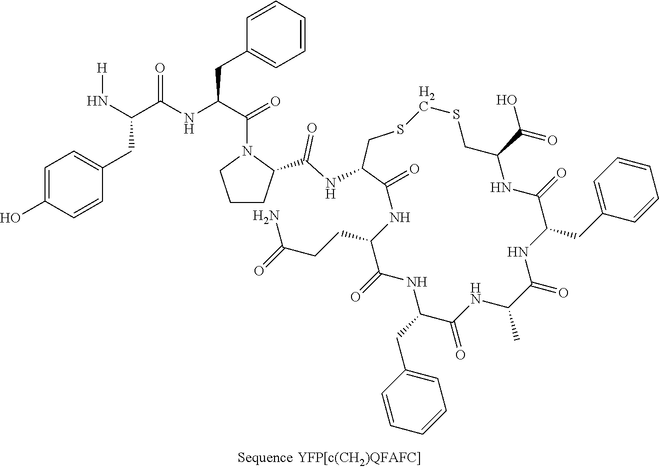 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CSCSC[C@@H](C(=O)O)NC(=O)[C@H](Cc2ccccc2)NC1=O]

After automated synthesis of YFPcQFAFC the peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Jupiter 4 μm Proteo 90 Å C12 column employing a gradient of 20-70% B in A over 40 min with a flow rate of 15 mL/min, detecting the peptide by measuring the absorption at λ=220 nm. The pure yield of the linear peptide was 5.3 mg (31% of theory). 0.6 mg of the peptide was dissolved in THF/H₂O (1:2) in the presence of 3 equiv K₂CO₃, 3 equiv TCEP, and 20 equiv Et₃N. This solution was added stepwise to a solution of 20 equiv CH₂I₂ in THF. The reaction was completed after shaking at rt for 12 h. The peptide was purified on a semipreparative a Kinetex 5 μm XB-C18 100 Å column employing a linear gradient of 30-60% B in A over 30 min with a flow rate of 5 mL/min. The pure yield of the cyclic peptide was 0.5 mg (83% of theory). Purity was determined by RP-HPLC on a Jupiter 4 μm Proteo 90 Å C12 employing a linear gradient of 20-70% B in A over 40 min with a flow rate of 1.0 mL/min. The peptide showed over 95% purity as determined by the absorption at 220 nm, with a retention time of 20.4 min. The chemical formula of the peptide is C₅₆H₆₈N₁₀O₁₂S₂ (monoisotopic mass: 1136.5 Da, average mass: 1137.3 Da). The observed masses were in correspondence to the calculated masses, confirming product identity: In MALDI-ToF, signals at m/z=1137.5 [M+H]⁺, 1159.5 [M+Na]⁺, and 1175.4 [M+K]⁺ were observed. In ESI ion trap, signals at m/z=1137.3 [M+H]⁺ and m/z=569.3 [M+2H]²⁺ were observed.

### Example 3: Tam-[c4,C9-TA]-chemerin-9 ($$ 8)

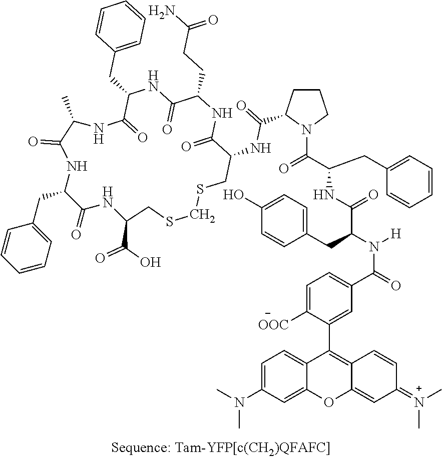

After automated synthesis of YFcGQFAFC, the N-terminus of the peptide was modified with 6-carboxytetramethylrhodamine (Tam) by reaction with 2 equiv of Tam, HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Kinetex 5 μm XB-C18 100 Å column employing a linear gradient of 30-80% B in A over 40 min, detecting the peptide by measuring the absorption at λ=220 nm. The pure yield of the linear peptide was 2.3 mg (10% of theory). The peptide was dissolved in THF/H₂O (1:2) in the presence of 3 equiv K₂CO₃, 3 equiv TCEP, and 20 equiv Et₃N. This solution was added stepwise to a solution of 20 equiv Ch₂I₂ in THF. The reaction was completed after shaking at rt for 12 h. The peptide was purified on a semi-preparative a Kinetex 5 μm XB-C18 100 Å column employing a linear gradient of 30-60% B in A over 30 min with a flow rate of 5 mL/min. The pure yield of the cyclic peptide was 0.73 mg (31% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 26.9 min and 22.6 min, respectively. The chemical formula of the peptide is C₈₁H₈₈N₁₂O₁₆S₂ (monoisotopic mass: 1548.6 Da, average mass: 1549.8 Da). The observed masses were in correspondence to the calculated masses, confirming product identity: In MALDI-ToF, a signal at m/z=1549.6 [M+H]⁺ was observed. In ESI ion trap, signals at m/z=1549.4 [M+H]⁺ and m/z=775.3 [M+2H]²⁺ were observed.

### Example 4: [c4,X9-TA]-chemerin-9 ($$ 9)

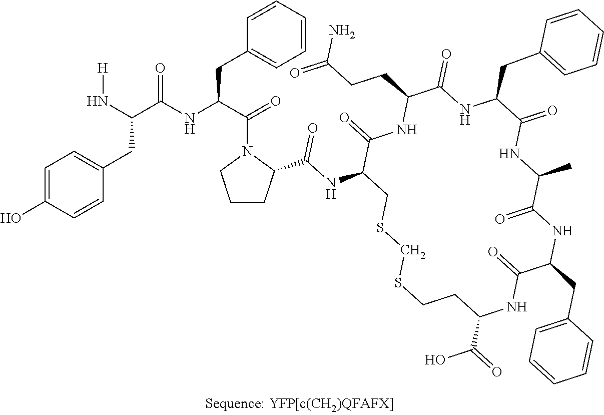 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CSCSCC[C@@H](C(=O)O)NC(=O)[C@H](Cc2ccccc2)NC1=O]

A 2-chlortrityl chloride resin was loaded with Fmoc-Homocysteine by reaction of the resin with 1.5 equiv of the amino acid and 5 equiv of DIPEA overnight. Loading was determined by cleaving the Fmoc-group off a defined amount of resin using piperidine and measuring the absorption of the piperidin-Fmoc adduct at λ=301 nm. After subsequent automated synthesis of YFPcQFAF, the peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Jupiter 4 μm Proteo 90 Å C12 column employing a gradient of 30-65% B in A over 35 min with a flow rate of 15 mL/min, detecting the peptide by measuring the absorption at λ=220 nm. The pure yield of the linear peptide was 1.3 mg (7.5% of theory). The peptide was dissolved in THF/H₂O (1:2) in the presence of 3 equiv K₂CO₃, 3 equiv TCEP, and 20 equiv Et₃N. This solution was added stepwise to a solution of 20 equiv CH₂I₂ in THF. The reaction was completed after shaking at rt for 12 h. The peptide was purified on a semi-preparative a Kinetex 5 μm XB-C18 100 Å column employing a linear gradient of 20-70% B in A over 40 min with a flow rate of 5 mL/min. The pure yield of the cyclic peptide was 0.7 mg (53% of theory). Purity was determined by RP-HPLC on a Jupiter 4 μm Proteo 90 Å C12 and on a Kinetex 5 μm XB-C18 100 Å column employing linear gradients of 20-70% B in A over 40 min with flow rates of 1.0 and 1.55 mL/min, respectively. The peptide showed over 95% purity as determined by the absorption at 220 nm, with a retention time of 20.9 min and 13.9 min, respectively. The chemical formula of the peptide is C₅₇H₇₀N₁₀O₁₂S₂ (monoisotopic mass: 1150.5 Da, average mass: 1151.4 Da). The observed masses were in correspondence to the calculated masses, confirming product identity: In MALDI-ToF, signals at m/z=1151.5 [M+H]⁺, 1173.5 [M+Na]⁺, and 1189.5 [M+K]⁺ were observed. In ESI ion trap, signals at m/z=1151.3 [M+H]⁺ and m/z=576.2 [M+2H]²⁺ were observed.

### Example 5: Tam-[c4,X9-TA]-chemerin-9 ($$ 10)

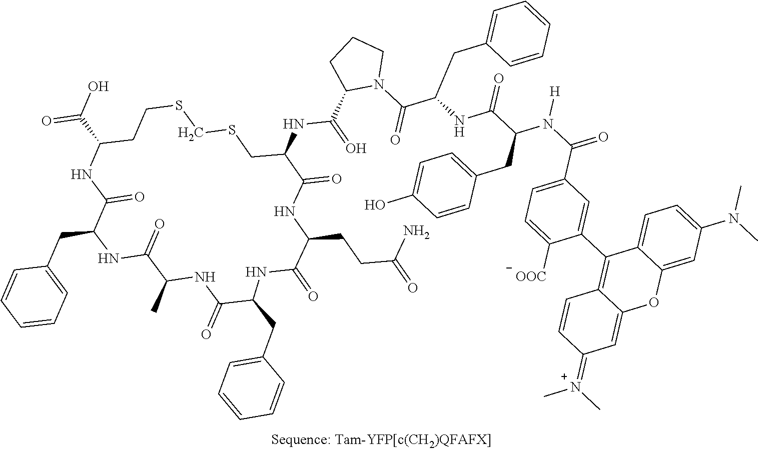

A 2-chlortrityl chloride resin was loaded with Fmoc-Homocysteine by reaction of the resin with 1.5 equiv of the amino acid and 5 equiv of DIPEA overnight. Loading was determined by cleaving the Fmoc-group off a defined amount of resin using piperidine and measuring the absorption of the pieridine-Fmoc adduct at λ=301 nm. After subsequent automated synthesis of YFPcQFAF, the N-terminus of the peptide was modified with 6-carboxytetramethylrhodamine (Tam) by reaction with 2 equiv of Tam. HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Kinetex 5 μm XB-C18 100 Å column employing a linear gradient of 30-80% B in A over 40 min, detecting the peptide by measuring the absorption at λ=220 nm. The pure yield of the linear peptide was 2.3 mg (10% of theory). The peptide was dissolved in THF/H₂O (1:2) in the presence of 3 equiv K₂CO₃, 3 equiv TCEP, and 20 equiv Et₃N. This solution was added stepwise to a solution of 20 equiv Ch₂I₂ in THF. The reaction was completed after shaking at rt for 12 h. The peptide was purified on a semi-preparative a Kinetex 5 μm XB-C18 100 Å column employing a linear gradient of 30-60% B in A over 30 min with a flow rate of 5 mL/min. The pure yield of the cyclic peptide was 0.73 mg (31% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 26.9 min and 22.6 min. respectively.

### Example 6: [y1,c4,X9-TA]-chemerin-9 ($$ 11)

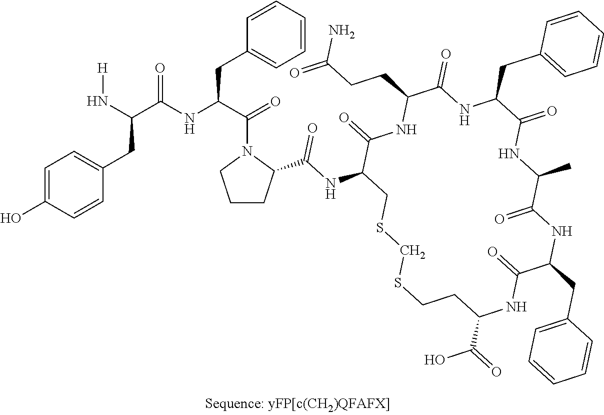 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](N)Cc2ccc(O)cc2)CSCSCC[C@@H](C(=O)O)NC(=O)[C@H](Cc2ccccc2)NC1=O]

A 2-chlortrityl chloride resin was loaded with Fmoc-Homocysteine by reaction of the resin with 1.5 equiv of the amino acid and 5 equiv of DIPEA overnight. Loading was determined by cleaving the Fmoc-group off a defined amount of resin using piperidine and measuring the absorption of the pieridine-Fmoc adduct at λ=301 nm. After subsequent automated synthesis of YFPcQFAF, the peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was purified by RP-HPLC on a Jupiter 4 μm Proteo 90 Å C12 column employing a gradient of 30-65% B in A over 35 min with a flow rate of 15 mL/min, detecting the peptide by measuring the absorption at λ=220 nm. The pure yield of the linear peptide was 2.5 mg (11% of theory). The peptide was dissolved in THF/H₂O (1:2) in the presence of 3 equiv K₂CO₃, 3 equiv TCEP, and 20 equiv Et₃N. This solution was added stepwise to a solution of 20 equiv CH₂I₂ in THF. The reaction was completed after shaking at rt for 12 h. The peptide was purified on a semi-preparative a Kinetex 5 μm XB-C18 100 Å column employing a linear gradient of 20-70% B in A over 40 min with a flow rate of 5 mL/min. The pure yield of the cyclic peptide was 0.8 mg (32% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 1.0 and 1.55 mL/min, respectively. The peptide showed over 95% purity as determined by the absorption at 220 nm, with retention times of 22.3 min and 12.9 min, respectively. The chemical formula of the peptide is C₅₇H₇₀N₁₀O₁₂S₂ (monoisotopic mass: 1150.5 Da, average mass: 1151.4 Da). The observed masses were in correspondence to the calculated masses, confirming product identity: In MALDI-ToF, a signal at m/z=1151.4 [M+H]⁺ was observed. In ESI ion trap, signals at m/z=1151.3 [M+H]⁺ and m/z=576.3 [M+2H]²⁺ were observed.

### Example 7: [y1,c4,K7(Tam),C9]-chemerin-9 ($$12)

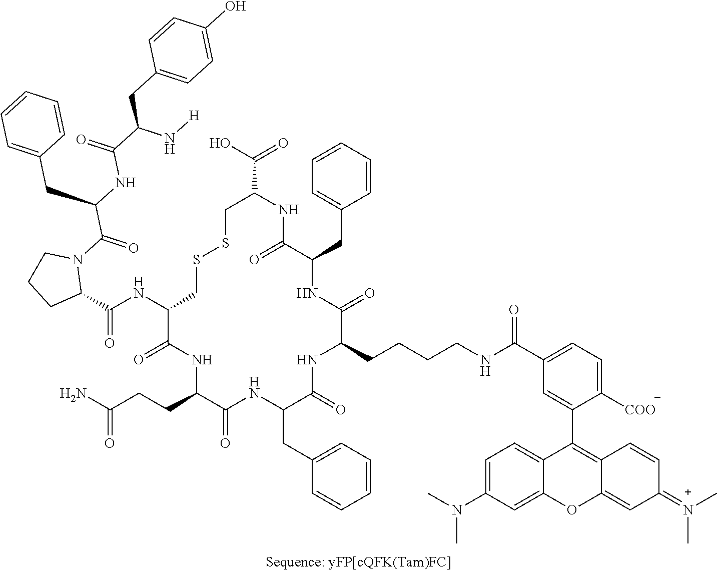

The peptide was synthesized incorporating a Dde-protected lysine at position 7 to allow selective modification of the peptide at the lysine said chain. After automated synthesis of yFPcQFK(Dde)FC, the Dde protecting group was cleaved by repeated reaction with 2% hydrazine in DMF, the reaction was monitored by measuring the UV absorbance of the Dde-hydrazine adduct at λ=300 nm. The peptide was modified with 6-carboxytetramethylrhodamine (Tam) by reaction with 2 equiv of Tam, HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed and the peptide was incubated in TBS, 20% ACN, pH 7.8 for 48 h to promote the formation of the intramolecular disulfide bond. The peptide was purified by RP-HPLC on a Aeris 3.6 μm 100 Å XB-C18 column employing a linear gradient of 30-60% B in A over 30 min, detecting the peptide by measuring the absorption at λ=220 nm. The yield of the pure peptide was 1.2 mg (5% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 1.0 and 1.55 mL/min, respectively. The peptide showed over 95% purity as determined by the absorption at 220 nm, with retention times of 24.1 min and 15.9 min, respectively. The chemical formula of the peptide is C₈₃H₉₃N₁₃O₁₆S₂ (monoisotopic mass: 1591.6 Da, average mass: 1592.9 Da). The observed masses were in correspondence to the calculated masses, confirming product identity: In MALDI-ToF, signals at m/z=1592.7 [M+H]⁺, m/z=1614.7 [M+Na]⁺, and at m/z=1630.6 [M+K]⁺ was observed. In ESI ion trap, signals at m/z=797.0 [M+2H]²⁺ and m/z=531.7 [M+3H]³⁺ were observed.

### Example 8: [x4,C9]-chemerin-9 ($$ 13)

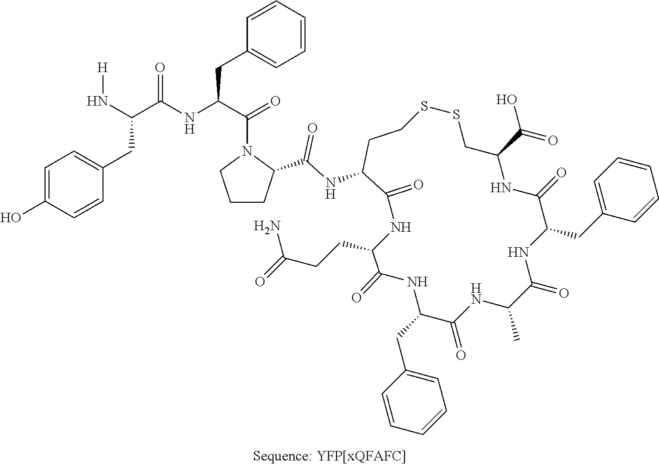 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CCSSC[C@@H](C(=O)O)NC(=O)[C@H](Cc2ccccc2)NC1=O]

After automated synthesis of QFAFC, D-homocysteine (x) was coupled by reaction of 5 equiv of HOBt, DIC and Fmoc-D-Homocysteine(Trt)-OH in DMF overnight, followed by automated synthesis of YFP. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Kinetex 5 μm XB-C18 100 Å column employing a gradient of 25 to 55% B in A over 30 min with a flow rate of 15 mL/min, detecting the peptide by measuring the absorption at λ=220n. The pure yield was 1.9 mg (11.1% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 20.5 min and 11.3 min, respectively. The chemical formula of the peptide is C₅₆H₆₈N₁₀O₁₂S₂ (monoisotopic mass: 1136.5 Da, average mass: 1137.3 Da). The observed masses were in correspondence to the calculated masses, confirming product identity: In MALDI-ToF, signals at m/z=1137.5 [M+H]⁺, at m/z=1159.5 [M+Na]⁺, and m/z=1175.5 [M+K]⁺ were observed. In ESI ion trap, signals at m/z=1137.2 [M+H]⁺ and m/z=569.2 [M+2H]²⁺ were observed.

### Example 9: [x4,W8,C9]-chemerin-9 ($$ 14)

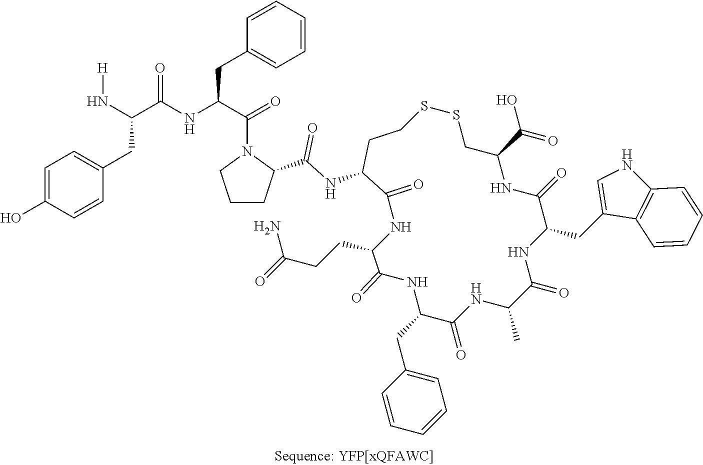 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CCSSC[C@@H](C(=O)O)NC(=O)[C@H](Cc2c[nH]c3ccccc23)NC1=O]

After automated synthesis of QFAWC, D-homocysteine (x) was coupled by reaction of 5 equiv of HOBt, DIC and Fmoc-D-Homocysteine(Trt)-OH in DMF overnight, followed by automated synthesis of YFP. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Jupiter 4 μm Proteo 90 Å C12 column employing a gradient of 30 to 60% B in A over 30 min with a flow rate of 15 mL/min. detecting the peptide by measuring the absorption at λ=220 nm. The pure yield was 2.0 mg (11.3% of theory). Purity was determined by RP-HPLC on a Jupiter 4 μm Proteo 90 Å C12 and on a Kinetex 5 μm biphenyl 100 Å column employing linear gradients of 20-70% B in A over 40 min with a flow rate of 1.0 and 1.55 mL/min, respectively. The peptide showed over 95% purity as determined by the absorption at 220 nm on both columns, with retention times of 20.6 min and 12.1 min, respectively. The chemical formula of the peptide is C₅₈H₆₉N₁₁O₁₂S₂ (monoisotopic mass: 1175.5 Da, average mass: 1176.4 Da). The observed masses were in correspondence to the calculated masses, confirming product identity: In MALDI-ToF, signals at m/z=1176.5 [M+H]⁺, and at m/z=1198.5 [M+Na]⁺ were observed. In ESI ion trap, signals at m/z=1176.2 [M+H]⁺ and m/z=588.8 [M+2H]²⁺ were observed.

### Example 10: [y1,x4,W8,C9]-chemerin-9 ($$ 15)

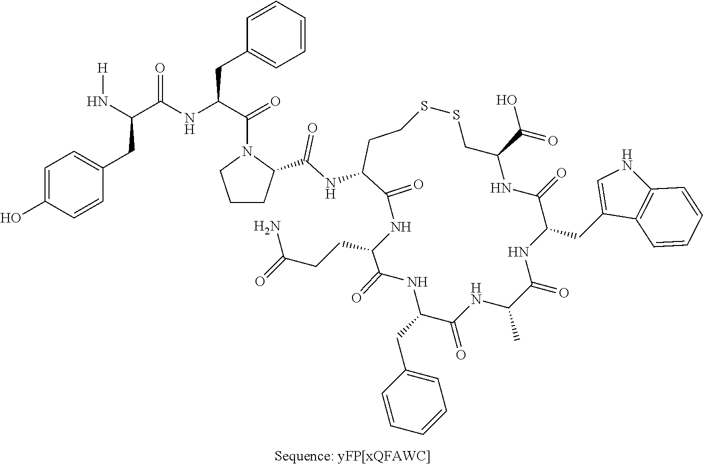 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](N)Cc2ccc(O)cc2)CCSSC[C@@H](C(=O)O)NC(=O)[C@H](Cc2c[nH]c3ccccc23)NC1=O]

After automated synthesis of QFAWC, D-homocysteine (x) was coupled by reaction of 5 equiv of HOBt, DIC and Fmoc-D-Homocysteine(Trt)-OH in DMF overnight, followed by automated synthesis of yFP. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Aeris 3.6 μm 100 Å XB-C18 column employing a gradient of 20 to 50% B in A over 30 min with a flow rate of 15 mL/min, detecting the peptide by measuring the absorption at λ=220 nm. The pure yield was 1.8 mg (10.2% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 0.6 and 1.55 mL/min, respectively. The peptide showed over 95% purity as determined by the absorption at 220 nm on both columns, with retention times of 22.7 min and 14.2 min, respectively. The chemical formula of the peptide is C₅₈H₆₉N₁₁O₁₂S₂ (monoisotopic mass: 1175.46 Da; average mass: 1176.38 Da). The observed masses were in correspondence to the calculated masses, confirming product identity: In MALDI-ToF a signal at m/z=1176.5 [M+H]⁺ was observed. In ESI ion trap, signals at m/z=1176.2 [M+H]⁺ and m/z=588.8 [M+2H]²⁺ were observed.

### Example 11: EG4-[x4,C9]-chemerin-9 ($$ 16)

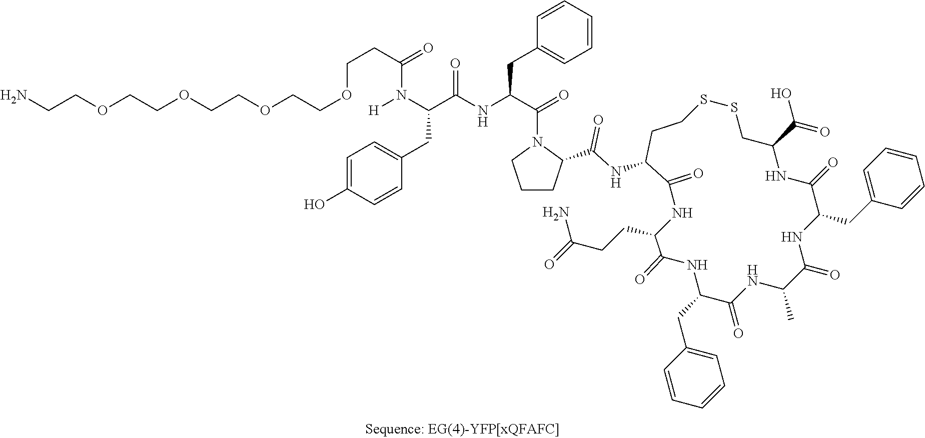 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](Cc2ccc(O)cc2)NC(=O)CCOCCOCCOCCOCCN)CCSSC[C@@H](C(=O)O)NC(=O)[C@H](Cc2ccccc2)NC1=O]

After automated synthesis of QFAFC, D-homocysteine (x) was coupled by reaction of 5 equiv of HOBt, DIC and Fmoc-D-Homocysteine(Trt)-OH in DMF overnight, followed by automated synthesis of YFP. EG(4) was coupled to the N-terminus of the peptide by reaction of 5 equiv of Fmoc-15-amino-4,7,10,13-tetraoxapentadecanoic acid, HOBt and DIC in DMF overnight. The Fmoc-group was cleaved by reaction with 20% piperidine in DMF for 10 min, the reaction was repeated twice. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Aeris 3.6 μm 100 Å XB-C18 column employing a gradient of 20 to 50% B in A over 30 min with a flow rate of 15 mL/min, detecting the peptide by measuring the absorption at λ=220 nm. The pure yield was 4.5 mg (21.6% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 0.6 and 1.55 mL/min, respectively. The peptide showed over 95% purity as determined by the absorption at 220 nm on both columns, with retention times of 21.9 min and 13.9 min, respectively. The chemical formula of the peptide is C₆₇H₈₉N₁₁O₁₇S₂ (monoisotopic mass: 1383.59 Da; average mass: 1384.63 Da). The observed masses were in correspondence to the calculated masses, confirming product identity: In MALDI-ToF a signal at m/z=1384.6 [M+H]⁺ was observed. In ESI ion trap, signals at m/z=1384.3 [M+H]⁺ and m/z=692.9 [M+2H]²⁺ were observed.

### Example 12: Tam-EG4-[x4,C9]-chemerin-9 ($$ 17)

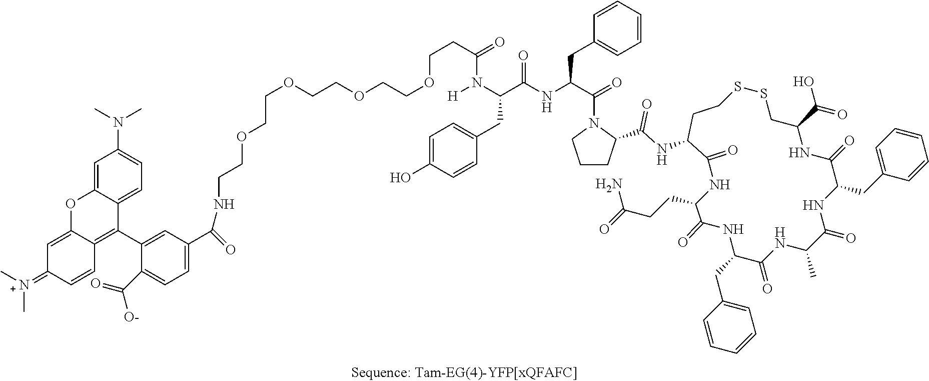 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](Cc2ccc(O)cc2)NC(=O)CCOCCOCCOCCOCCNC(=O)c2ccc(C(=O)[O-])c(-c3c4ccc(=[N+](C)C)cc-4oc4cc(N(C)C)ccc34)c2)CCSSC[C@@H](C(=O)O)NC(=O)[C@H](Cc2ccccc2)NC1=O]

After automated synthesis of QFAFC, D-homocysteine (x) was coupled by reaction of 5 equiv of HOBt, DIC and Fmoc-D-Homocysteine(Trt)-OH in DMF overnight, followed by automated synthesis of YFP. EG(4) was coupled to the N-terminus of the peptide by reaction of 5 equiv of Fmoc-15-amino-4,7,10,13-tetraoxapentadecanoic acid, HOBt and DIC in DMF overnight. The Fmoc-group was cleaved by reaction with 20% piperidine in DMF for 10 min, the reaction was repeated twice. The peptide was N-terminally modified with 6-carboxytetramethylrhodamine (Tam) by reaction with 2 equiv of Tam. HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Aeris 3.6 μm 100 Å XB-C18 column employing a gradient of 20 to 50% B in A over 30 min with a flow rate of 15 mL/min, detecting the peptide by measuring the absorption at λ=220 nm. The pure yield was 1.8 mg (6.7% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 0.6 and 1.55 mL/min, respectively. The peptide showed over 95% purity as determined by the absorption at 220 nm on both columns, with retention times of 22.9 min and 14.9 min, respectively. The chemical formula of the peptide is C₉₂H₁₀₉N₁₃O₂₁S₂ (monoisotopic mass: 1795.73 Da; average mass: 1797.07 Da). The observed masses were in correspondence to the calculated masses, confirming product identity: In MALDI-ToF a signal at m/z=1796.7 [M+H]⁺ was observed. In ESI ion trap, signals at m/z=1797.3 [M+H]⁺ and m/z=599.7 [M+2H]²⁺ were observed.

### Example 13: EG4-[x4,W8,C9]-chemerin-9 ($$ 18)

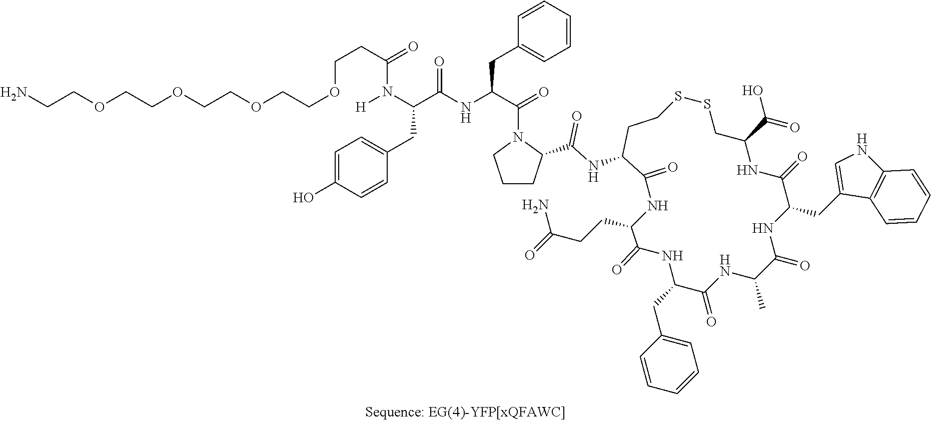 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](Cc2ccc(O)cc2)NC(=O)CCOCCOCCOCCOCCN)CCSSC[C@@H](C(=O)O)NC(=O)[C@H](Cc2c[nH]c3ccccc23)NC1=O]

After automated synthesis of QFAWC, D-homocysteine (x) was coupled by reaction of 5 equiv of HOBt, DIC and Fmoc-D-Homocysteine(Trt)-OH in DMF overnight, followed by automated synthesis of YFP. EG(4) was coupled to the N-terminus of the peptide by reaction of 5 equiv of Fmoc-15-amino-4,7,10,13-tetraoxapentadecanoic acid, HOBt and DIC in DMF overnight. The Fmoc-group was cleaved by reaction with 20% piperidine in DMF for 10 min, the reaction was repeated twice. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN, pH 7.8 for 48 h to promote the formation of the intramolecular disulfide bond. The peptide was purified by RP-HPLC on a Aeris 3.6 μm 100 Å XB-C18 column employing a gradient of 20 to 50% B in A over 30 min with a flow rate of 15 mL/min, detecting the peptide by measuring the absorption at λ=220 nm. The pure yield was 4.6 mg (22.2% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 0.6 and 1.55 mL/min, respectively. The peptide showed over 95% purity as determined by the absorption at 220 nm on both columns, with retention times of 21.4 min and 13.4 min, respectively. The chemical formula of the peptide is C₆₇H₈₉N₁₁O₁₇S₂ (monoisotopic mass: 1383.59 Da; average mass: 1384.63 Da). The observed masses were in correspondence to the calculated masses, confirming product identity: In MALDI-ToF a signal at m/z=1384.6 [M+H]⁺ was observed. In ESI ion trap, signals at m/z=1384.3 [M+H]⁺ and m/z=692.9 [M+2H]²⁺ were observed.

### Example 14: Tam-EG4-[x4,W8,C9]-chemerin-9 ($$19)

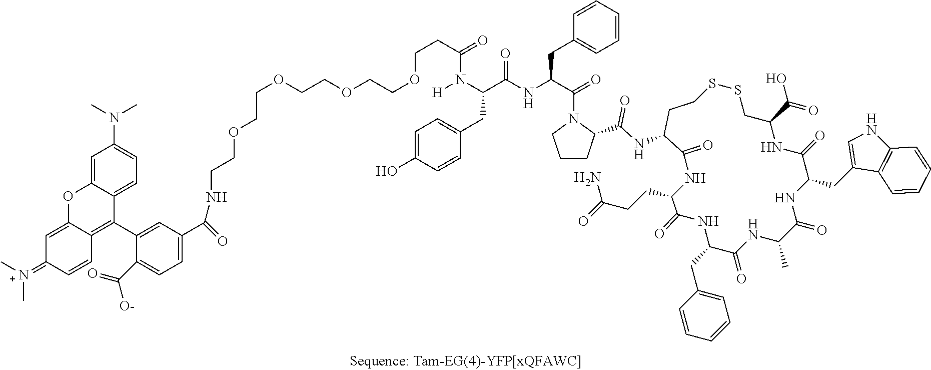 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](Cc2ccc(O)cc2)NC(=O)CCOCCOCCOCCOCCNC(=O)c2ccc(C(=O)[O-])c(-c3c4ccc(=[N+](C)C)cc-4oc4cc(N(C)C)ccc34)c2)CCSSC[C@@H](C(=O)O)NC(=O)[C@H](Cc2c[nH]c3ccccc23)NC1=O]

After automated synthesis of QFAFC, D-homocysteine (x) was coupled by reaction of 5 equiv of HOBt, DIC and Fmoc-D-Homocysteine(Trt)-OH in DMF overnight, followed by automated synthesis of YFP. EG(4) was coupled to the N-terminus of the peptide by reaction of 5 equiv of Fmoc-15-amino-4,7,10,13-tetraoxapentadecanoic acid, HOBt and DIC in DMF overnight. The Fmoc-group was cleaved by reaction with 20% piperidine in DMF for 10 min, the reaction was repeated twice. The peptide was N-terminally modified with 6-carboxytetramethylrhodamine (Tam) by reaction with 2 equiv of Tam, HATU and DIPEA in DMF overnight. The peptide was treated with 90% TFA, 7% thioanisol, 3% ethanedithiol for 3 h to deprotect all side chains and cleave the peptide from the resin, followed by precipitation in ice-cold Et₂O/hexane (1:3). The precipitate was washed with Et₂O and the peptide was incubated in TBS, 20% ACN. pH 7.8 for 48 h to promote the formation of the intramolecular disulfide bond. The peptide was purified by RP-HPLC on a Aeris 3.6 μm 100 Å XB-C18 column employing a gradient of 20 to 50% B in A over 30 min with a flow rate of 15 mL/min, detecting the peptide by measuring the absorption at λ=220 nm. The pure yield was 1.9 mg (6.69% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 0.6 and 1.55 mL/min, respectively. The peptide showed over 95% purity as determined by the absorption at 220 nm on both columns, with retention times of 22.4 min and 14.4 min, respectively. The chemical formula of the peptide is C₉₂H₁₀₉N₁₃O₂₁S₂ (monoisotopic mass: 1795.73 Da; average mass: 1797.07 Da). The observed masses were in correspondence to the calculated masses, confirming product identity: In MALDI-ToF a signal at m/z=1796.7 [M+H]⁺ was observed. In ESI ion trap, signals at m/z=1797.3 [M+H]⁺ and m/z=599.7 [M+2H]²⁺ were observed.

### Example 15: GFLG-[x4,C9]-chemerin-9 ($$ 20)

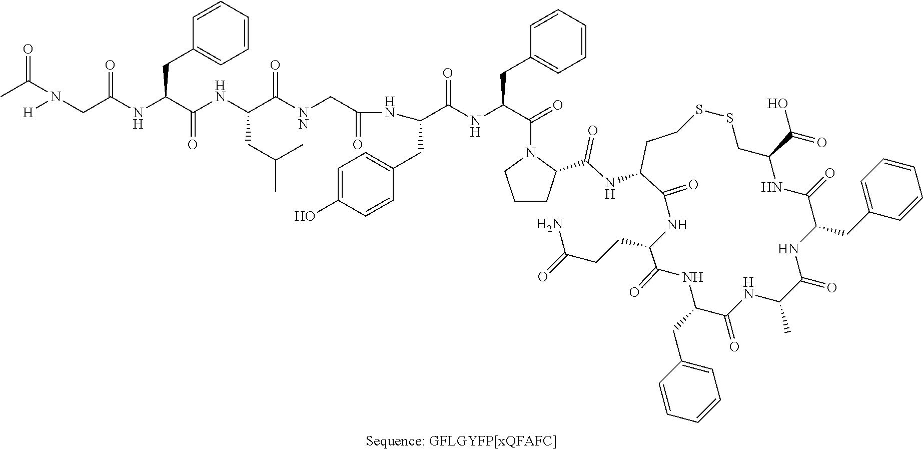

After automated synthesis of QFAFC, D-homocysteine (x) was coupled by reaction of 5 equiv of HOBt, DIC and Fmoc-D-Homocysteine(Trt)-OH in DMF overnight, followed by automated synthesis of GFLGYFP. The N terminus of the peptide was acetylated on resin with Ac₂O and DIPEA in DCM for 15 min. The peptide was treated with 90% TFA, 7% thioanisol, 3% ethanedithiol for 3 h to deprotect all side chains and cleave the peptide from the resin, followed by precipitation in ice-cold Et2O/hexane (1:3). The precipitate was washed with Et2O and the peptide was incubated in TBS, 20% ACN, pH 7.8 for 48 h to promote the formation of the intramolecular disulfide bond. The peptide was purified by RP-HPLC on a Aeris 3.6 μm 100 Å XB-C18 column employing a gradient of 20 to 50% B in A over 30 min with a flow rate of 15 mL/min, detecting the peptide by measuring the absorption at λ=220 nm. The pure yield was 1.7 mg (7.3% of theory). Purity was determined by RP-HPLC on a Jupiter 4 μm Proteo 90 Å C12 and on a Aeris 3.6 μm 100 Å XB-C18 column employing linear gradients of 20-70% B in A over 40 min with flow rates of 0.6 and 1.55 mL/min, respectively. The peptide showed over 95% purity as determined by the absorption at 220 nm on both columns, with retention times of 22.5 min and 14.1 min, respectively. The chemical formula of the peptide is C₇₇H₉₆N₁₄O₁₇S₂ (monoisotopic mass: 1552.65 Da; average mass: 1553.82 Da). The observed masses were in correspondence to the calculated masses, confirming product identity: In MALDI-ToF a signal at m/z=1575.6 [M+Na]⁺ was observed. In ESI ion trap, signals at m/z=1554.6 [M+H]+ and m/z=777.3 [M+2H]2+ were observed.

### III Experimental Section—Biological Assays

### Test Systems and Methods

### Cell Culture

COS-7 and HEK293 cells were cultivated in DMEM supplemented with 10% FBS or DMEM/Ham's F12 supplemented with 15% FBS, respectively. All cells were maintained in T75 cell culture flasks at 37° C., 95% humidity and 5% CO₂ (standard conditions).

### 1. Plasma Stability Assay

Investigation of peptide stability in blood plasma was carried out as described previously. (Hoppenz, Els-Heindl et al., A Selective Carborane-Functionalized Gastrin-Releasing Peptide Receptor Agonist as Boron Delivery Agent for Boron Neutron Capture Therapy. J Org Chem, 2020, 85(3): 1446-1457) Tam-labeled peptides were dissolved in human blood plasma at a concentration of 10⁻⁵ M and incubated at 37° C. and 250 rpm. Samples taken at the respective time points were added to a solution of 0.1% SDS in ACN/EtOH (1:1). After incubation at −20° C. for 20 min, the supernatant was transferred to a new tube and incubated again at −20° C. for at least 3 h. The solution was filtered by centrifugation using Costar Spin-X tubes (0.22 μm) and the filtrate was analyzed by RP-HPLC on a VariTide RPC. 6 μm, 200 Å column (Agilent technologies, Santa Clara, USA) employing a linear gradient of 15-65% (v/v) A in B over 40 min. The fluorescence of the peptide was detected at λ=573 nm. Peaks were integrated and the peak containing the intact peptide was normalized to the sample taken at t=0 min (100%). Plasma half-life was determined using one-phase decay in GraphPad Prism 5.03.

### 2. Calcium Mobilization Assay

COS-7 cells were transfected in 75 cm² cell culture flasks with 12 μg of the hCMKLR1_eYFP_G_{αΔ6qi4myr}_pV2 plasmid overnight using Metafectene Pro. Transfected cells were seeded in 96 well plates (100 μL cell suspension in DMEM+10% FBS/well) and incubated overnight. The following day, the Ca²⁺-mobilization was performed as described previously. (Hoppenz, Els-Heindl et al., A Selective Carborane-Functionalized Gastrin-Releasing Peptide Receptor Agonist as Boron Delivery Agent for Boron Neutron Capture Therapy. J Org Chem, 2020, 85(3): 1446-1457) Briefly, cells were incubated with Fluo-2-AM solution (2.3 μM Fluo-2-AM, 0.06% (v/v) Pluronic-F127 in assay buffer). After 1 h, the Fluo-2-am solution was replaced with assay buffer (20 mM HEPES, 2.5 mM Probenecid in HBSS, pH 7.5) and the basal Ca²-level was measured for 20 s with a Flexstation 3 (λₑₓ=485 nm, λₑₘ=525 nm). The ligand was added, and Ca²⁺-response was measured for 40 s. The resulting maximum over basal values were calculated for each well, and normalized to the top and bottom values of the control curve (chemerin-9 $$1). All experiments were performed in triplicates, each experiment was repeated at least twice. Nonlinear regression was calculated using GraphPad Prism 5.

### 3. Bioluminescence Resonance Energy Transfer (BRET)

HEK293 cells were transiently transfected using 75 cm² cell culture flasks with cell monolayers (confluency of ˜80%). Plasmid DNA of the C-terminally eYFP tagged human CMKLR1 and the chimeric G protein G_{αΔ6qi4myr} in a pVitro2 vector (7.8 μg) and Renilla-luciferase 8-tagged Arrestin 3 in pcDNA3 (0.2 μg) and 24 μl MetafectenePro were separately added to 900 μl DMEM/Ham's F12 and incubated for 10 min before unification and incubation at RT for 20 min. 6 ml DMEM/Ham's F12 with 15% FCS at 37° C. was added on the cell monolayer. The plasmid solution was added and cells were incubated overnight before seeding. Cell seeding was carried out in white 96-well polystyrene cell culture microplates, coated with poly-D lysine. Transfected cells were detached with 1 ml trypsin/EDTA, 21 ml DMEM/Ham's F12 with 15% FCS was added and 100,000-200.000 cells in 100 μl per well were seeded. Afterwards, the cells were incubated overnight at 37° C. The assay was performed under unsterile conditions. First, medium was displaced with 100 μl BRET buffer (HBSS, 25 mM HEPES. pH 7.3) and 50 μl of luciferase substrate coelenterazine-h (final concentration of 4.2 μM) was added. Afterwards, the cells were stimulated with the peptides in different concentrations (10⁻⁵ to 10⁻¹² M) dissolved in BRET buffer. 50 μl of the peptide dilution were used for cell stimulation. Buffer without peptide was used as a negative control. BRET effect was measured 15 min after agonist addition with a Tecan infinite plate reader using two filter sets at 37° C. (luminescence filter 400 nm-470 nm and fluorescence filter 505 nm-590 nm) and plotted as a function of fluorescence/luminescence ratio. The values of the negative control were subtracted, non-linear regression was calculated using GraphPad Prism. The curves were normalized to the positive control wild type chemerin 9 ($$1). All measurements were performed in four technical replicates, all experiments were repeated at least twice.

### 4. Fluorescence Microscopy

Cellular arrestin 3 recruitment was verified and CMKLR1 receptor uptake was tested in HEK293 cells. Ibidi 15μ-slides were coated with poly D-lysine before cell seeding. Cells were washed with DPBS prior to detachment with 1 ml trypsin/EDTA. A Neubauer chamber was used to count the amount of cells/ml medium after addition of 9 ml DMEM/Ham's F12 with 15% FCS. The cell suspension was diluted to 140,000 cells/200 μl, which were seeded. Incubation was carried out overnight at 37° C. Afterwards, cells were transiently transfected. Plasmid DNA of hCMKLR1_eYFP_G_{αΔ6qi4myr}_pV2 (0.9 μg) and mCherry_Arr3_pcDNA3 (0.1 μg) and 8 μl Lipofectamine were separately added to 100 μl DMEM/Ham's F12 and incubated for 10 min before unification and incubation at RT for 20 min. Incubation was carried out overnight at 37° C. Then, the cells were starved with 200 μl OptiMEM and 1 μl of Hoechst 33342 for 30 min. The medium was replaced by 200 μl of OptiMEM and the to status was documented. The OptiMEM was then replaced by 200 μl of 1 μM peptide in OptiMEM. Fluorophore excitation was analyzed using different filters, depending on the emission wavelength of the fluorophore and the time of exposure was adjusted to each fluorophore individually. All images were processed identically with the AxioVision software (Carl Zeiss AG, Oberkochen, Germany).

TABLE 1
Filter sets (Zeiss) used for fluorophore detection.

| Fluorophore | Used for labeling | Excitation [nm] | Emission [nm] |
|---|---|---|---|
| Hoechst33342 | cell nucleus | 352 | 455 |
| mCherry | arrestin 3 | 549 | 577 |
| YFP | hCMKLR1 | 514 | 526 |

Hoechst33342: 2′-(4-Ethoxyphenyl)-6-(4-methyl-1-piperazinyl)-1H,3′H-2,5′-bibenzimidazole; YFP: yellow fluorescent protein; hCMKLR1: chemokine like receptor 1;

### Results

### Activity Studies in Ca²⁺ Mobilization Assay

To test the ability of the synthesized peptides to induce G protein-mediated signaling at the CMKLR1, a Ca²⁺ mobilization assay was performed. All tested cyclic chemerin-9 variants showed G protein-signaling to various extends (Table 2). The most active cyclic derivate Example 8: [x4,C9]-chemerin-9 ($$ 13) showed a 2-fold higher activity than linear chemerin-9 (comparison example 1). Introducing a tryptophan in position 8 increased the activity even more ($$14), but combining modifications Trp⁸ and D-Tyr¹ in one peptide led to a significantly decreased activity ($$15).

TABLE 2
Activity data of chemerin-9 derivates at
the CMKLR1 in a Ca²⁺ mobilization assay.

| Cpd | Code | EC₅₀ [nM] | pEC50 ± SEM |
|---|---|---|---|
| $$ 1 | Comparison Example 1: Chemerin-9 ($$ 1) | 10 | 8.021 ± 0.039 |
| $$ 4 | Comparison Example 4: [c4,C9]-chemerin-9 ($$ 4) | 64 | 7.192 ± 0.147 |
| $$ 6 | Comparison Example 5: [c4,C7]-chemerin-9 ($$6) | 63 | 7.204 ± 0.106 |
| $$ 7 | Example 2: [c4,C9-TA]-chemerin-9 ($$ 7) | 13 | 7.888 ± 0.130 |
| $$ 9 | Example 4: [c4,X9-TA]-chemerin-9 ($$ 9) | 37 | 7.429 ± 0.110 |
| $$ 11 | Example 6: [y1,c4,X9-TA]-chemerin-9 ($$ 11) | 28 | 7.553 ± 0.067 |
| $$ 13 | Example 8: [x4,C9]-chemerin-9 ($$ 13) | 5 | 8.259 ± 0.074 |
| $$ 14 | Example 9: [x4,W8,C9]-chemerin-9 ($$ 14) | 3 | 8.499 ± 0.058 |
| $$ 15 | Example 10: [y1,x4,W8,C9]-chemerin-9 ($$ 15) | 207 | 6.683 ± 0.101 |
| $$ 16 | Example 11: EG4-[x4,C9]-chemerin-9 ($$16) | 17 | 7.766 ± 0.226 |
| $$ 18 | Example 13: EG4-[x4,W8,C9]-chemerin-9 ($$ 18) | 193 | 6.714 ± 0.088 |
| $$ 20 | Example 15: GFLG-[x4,C9]-chemerin-9 ($$ 20) | 21 | 7.676 ± 0.221 |

X = Homocysteine, x = D-Homocysteine, TA = thioacetal.

### Plasma Stability Studies

The stability of the different peptides in blood plasma was investigated for Tam-modified derivates to enable monitoring the degradation of the peptides in RP-HPLC. All N-terminally Tam-labeled cyclic chemerin-9 variants ($$5, $$8, $$9) were completely stable in blood plasma over a time period of 24 h. Introducing the Tam-label at a side chain while simultaneously inducing an N-terminal D-Tyr gave the equally stable Example 7: [y1,c4,K7(Tam),C9]-chemerin-9 ($$12). This demonstrates that all cyclic variants with an N-terminal stabilization can be expected to be stable in blood plasma. This was verified by testing the chemerin-9 variants bearing an N-terminal ethylene glycol linker ($$17, $$19), both were completely stable for at least 48 h).

TABLE 3
Plasma stability of Tam-modified chemerin-9 derivates.

| Cpd | Code | t_{1/2} [h] |
|---|---|---|
| $$ 2 | Comparison Example 2: Tam-chemerin-9 ($$ 2) | <0.2 |
| $$ 5 | Example 1: Tam-[c4,C9]-chemerin-9 ($$ 5) | >24 |
| $$ 8 | Example 3: Tam-[c4,C9-TA]-chemerin-9 ($$ 8) | >24 |
| $$ 10 | Example 5: Tam-[c4,X9-TA]-chemerin-9 ($$ 10) | >24 |
| $$ 12 | Example 7: [y1,c4,K7(Tam),C9]-chemerin-9 ($$ 12) | >24 |
| $$ 17 | Example 12: Tam-EG4-[x4,C9]-chemerin-9 ($$ 17) | >48 h |
| $$ 19 | Example 14: Tam-EG4-[x4,W8,C9]-chemerin-9 ($$ 19) | >48 h |

### Internalization Studies in BRET and Microscopy

Arrestin recruitment is the first step in the internalization process of GPCR that follows activation of the G protein pathway. The bioluminescence resonance energy transfer (BRET) assay was used to determine the potency of cyclic chemerin variants to recruit arrestin 3 to the CMKLR1 receptor after stimulation. HEK293 cells were transiently transfected with CMKLR1 receptor fused with eYFP fluorophore and Arrestin 3 tagged with Rluc8 luciferase. The transfected cells were seeded, incubated with the luciferase substrate coelenterazine-h and stimulated with different peptide concentrations, resulting in measurable BRET signals. The sigmoidal concentration-response-curves were normalized to the positive control WT chemerin 9 ($$1). In the BRET assays, it could be shown that all but one of the tested peptides are still able to induce arrestin 3 recruitment to the activated CMKLR1 receptor with different potencies (Table 1).

TABLE 1
Arrestin recruitment data of selected peptides obtained in a BRET
based assay. All data was obtained from at least two individual experiments
and normalized to the control compound chemerin-9 ($$ 1).

| | | EC₅₀ | pEC₅₀ ± | Eₘₐₓ |
|---|---|---|---|---|
| Cpd | Code | [nM] | SEM | [%] ± SEM |
| ($$ 1) | Comparison Example 1: Chemerin-9 ($$ 1) | 37 | 7.4 ± 0.05 | 96 ± 2 |
| ($$ 13) | Example 8: [x4,C9]-chemerin-9 ($$ 13) | 78 | 7.1 ± 0.1 | 86.8 ± 4 |
| ($$ 14) | Example 9: [x4,W8,C9]-chemerin-9 ($$14) | 158 | 6.8 ± 0.2 | 97 ± 10 |
| ($$ 15) | Example 10: [y1,x4,W8,C9]-chemerin-9 ($$ 15) | — | — | — |
| ($$ 16) | Example 11: EG4-[x4,C9]-chemerin-9 ($$ 16) | 101 | 6.8 ± 0.08 | 102 ± 5 |
| ($$ 18) | Example 13: EG4-[x4,W8,C9]-chemerin-9 ($$18) | 418 | 6.4 ± 0.2 | 69 ± 8 |
| ($$ 20) | Example 15: GFLG-[x4,C9]-chemerin-9 ($$ 20) | 100 | 7.0 ± 0.2 | 89 ± 5 |

x = D-homocysteine

Cyclization of chemerin 9 by a disulfide bond ($$13) leads to only slightly shifted EC₅₀ and Eₘₐₓ values compared with wild type chemerin 9. An additional exchange at position 8 to tryptophan is also accepted ($$14). Interestingly, a further modification with D-tyrosine at position 1 leads to a peptide that is no longer able to induce arrestin 3-recruitment, despite its activity in G protein activation in a Ca²⁺ assay ($$15), making it a biased ligand. Elongation of the N-terminus of cyclic peptides with an ethylene glycol linker ($$16, $$18) or short peptide linker ($$20) is accepted with respect to arrestin-recruitment.

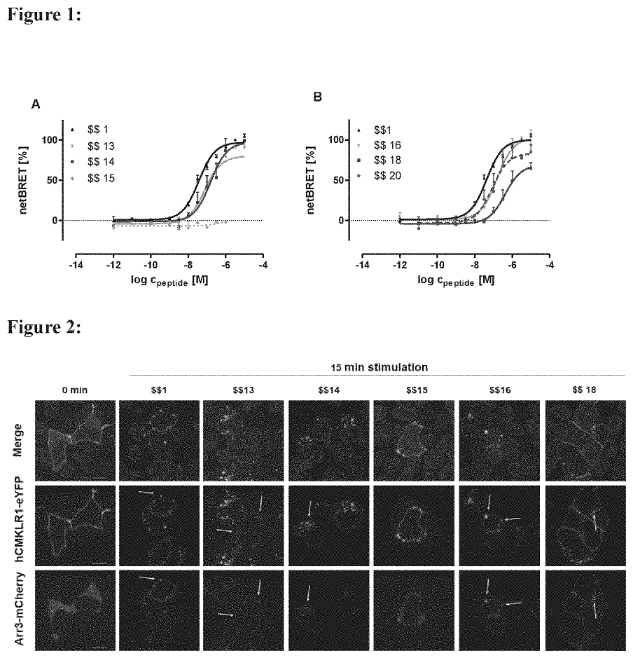

FIG. 1: Arrestin 3 recruitment to the CMKLR1 after stimulation with chemerin-9 variants. Impact of cyclization and amino acid substitutions on the activity chemerin-9

Impact of cyclization and amino acid substitutions on the activity chemerin-9 is shown in FIG. 1. A) Cyclization ($$13) leads to a slight loss of activity compared to linear chemerin-9 ($$1). Exchanging position 8 for a tryptophan ($$14) has no impact, while changing position 8 for tryptophan and position 1 for D-tyrosine completely abolishes arrestin recruitment ($$15). B) N-terminal elongation of cyclic peptides with either polyethylene glycol or peptide linker has no impact ($$16, $$ 20), unless a tryptophane is present in position 8 ($$18).

To verify the arrestin-recruitment and analyze the internalization of the CMKLR1 receptor itself, HEK293 cells were used and transiently transfected with fluorescent labeled variants of the two molecules. These cells express the human CMKLR1 fused to a C terminally yellow fluorescent protein (YFP) and arrestin 3 with a red fluorescent mCherry protein.

FIG. 2: Internalization of CMKLR1 and arrestin 3 recruitment. HEK293 cells expressing the hCMKLR1 (green) and arrestin 3 (Arr3; red) due to transient transfection were used for the internalization studies. Inter-nalization of the receptor was observed for 30 min stimulation with 1 μM via fluorescence microscopy after nuclei staining (blue). Representative pic-tures for time point 15 min were chosen and processed identically with the AxioVision software; scale bar: 15 μm; n≥2;

Without stimulation, the receptor was located at the cell membrane and the arrestin was distributed in the cytosol (FIG. 2, 0 min). After stimulation with chemerin-9 (comparison example 1, $$1), arrestin 3 was recruited to the CMKLR1 receptor followed by internalization of the CMKLR1-arrestin complex. A similar behavior was demonstrated for the cyclic variant Example 8: [x4,C9]-chemerin-9 ($$13), Example 9: [x4,W8,C9]-chemerin-9 ($$14) and Example 11: EG4-[x4,C9]-chemerin-9 ($$16). Example 13: EG4-[x4,W8,C9]-chemerin-9 ($$18) shows good arrestin 3 recruitment, but slightly lower receptor internalization compared to chemerin-9 (comparison example 1, $$1). In contrast, neither internalization of the hCMKLR1 nor arrestin 3 recruitment could be detected for Example 10: [y1,x4,W8,C9]-chemerin-9 ($$15). Thus, the bias behavior of this compound was verified. These results obtained in fluorescence microscopy confirm the results obtained in the BRET-based assay for all peptides.

1: A compound of formula (I): 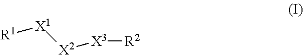
wherein
R¹ is absent
or
represents 6-Carboxytetramethylrhodamine (Tam), ##C(O)R³, C₈-C₂₀ fatty acid or the sequence R⁴GFLG ##, R⁴—C═N—NH-##, R⁴—S—S-##, R⁴—N═N-##, R-Valin-Citrullin-##, R⁴—C(O)O-## or R⁴NH—C(O)O-##
wherein
## marks the attachment to the terminal amino group of X¹,
R³ represents C₁-C₆-alkylene, aryl, heteroaryl, C₃-C₈-cycloalkyl or C₃-C₇-heterocycloalkyl,
wherein C₁-C₆-alkylene is up to trisubstituted identically or differently by a radical selected from the group consisting of hydroxyl, methoxy, ethoxy, carboxy, amino and halogen,
wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
R⁴ represents 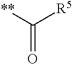
wherein
R⁵ represents C₁-C₆-alkylene, aryl, heteroaryl, C₃-C₈-cycloalkyl or C₃-C₇-heterocycloalkyl,
wherein C₁-C₆-alkylene is up to trisubstituted identically or differently by a radical selected from the group consisting of hydroxyl, methoxy, ethoxy, carboxy, amino and halogen,
wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
or
represents a group of the formula (IIIa) 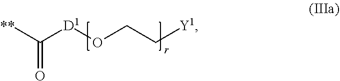
wherein
** marks the attachment to a nitrogen atom,
D¹ is C₁-C₄-alkylene,
Y¹ is selected from the group consisting of hydroxyl, methoxy, ethoxy, carboxy, carboxamide or amino
wherein amino might be substituted with 6-carboxytetramethyirhodamine (Tam) via an amide bond,
and
r represents an integer of from 2 to 15,
R² represents a group of the formula (II) 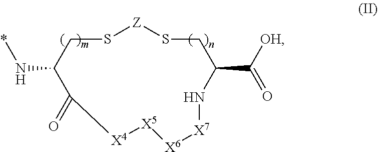
or
represents a group of the formula (III) 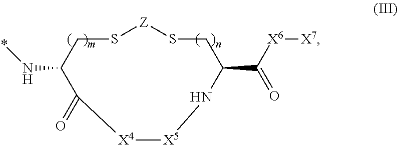
wherein
* represents the attachment to the carbonyl atom of the carboxy group of X³,
Z represents a bond or —CH₂—,
m represents 1 or 2,
n represents 1 or 2,
X¹ represents a natural amino acid selected from a list consisting of L, I, F, H, M, W, Y or y or an unnatural amino acid selected from a list consisting of L-Norleucine (Nle), 2,3,3a,4,5,6,7,7a-Octahydroindole-2-carboxylic acid (Oic), 4-Bromophenylalanine ((4-Bromo)F), 2,5-Difluorophenylalanine ((2,5-Difluoro)F), 2-Chlorophenylalanine ((2-Chlor)F), 3-Chlorophenylalanine ((3-Chlor)F), 4-Chlorophenylalanine ((4-Chloro)F), 2-Bromophenylalanine ((2-Bromo)F), 3-Bromophenylalanine ((3-Bromo)F), 4-Bromophenylalanine ((4-Bromo)F), 2-Fluorophenylalanine ((2-Fluoro)F), 3-Fluorophenylalanine ((3-Fluoro)F), 4-Fluorophenylalanine ((4-Fluoro)F), (2,5-difluoro-phenylalanine, 2-Methylphenylalanine ((2-Me)F), 3-Methyl-phenylalanine ((3-Me)F), 4-Methylphenylalanine ((4-Me)F), (2S)-3-(2,3-difluorophenyl)-2-aminopropanoic acid, Phenylglycine (Phg)N-Phenylglycine ((N-Ph)G), 3-Chlorophenylglycine ((3-Chloro-Ph)G), 3-(1,3-Benzothiazol-2-yl)-alanine 1-Benzyl-histidine (H(1-Bn)), 1-Methyl-histidine (H(1-Me)), 3-Methylhistidine (3-Me)H), 2-Pyridylalanine (2-Pal), 3-Pyridylalanine (3-Pal), 4-Pyridylalanine (4-Pal), 3-(Aminomethyl)benzoic acid, 1-Napthylalanine (1-Nal), 2-Napthylalanine (2-Nal), (2R)-Amino-(1-methyl-1H-indazol-5-yl)acetic acid and (2S)-3-(indol-4-yl)-2-(amino)propanoic acid, whereas any natural amino acid and/or unnatural amino acid from that list can be in D- or L-stereoconfiguration,
X² represents a natural amino acid selected from a list consisting of L, I, F, H, M, W or Y or an unnatural amino acid selected from a list consisting of L-Norleucine (Nle), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Napthylalanine (1-Nal), L-2-Napthylalanine (2-Nal), (2R)-Amino-(1-methyl-1H-indazol-5-yl)acetic acid and (2S)-3-(indol-4-yl)-2-(amino)propanoic acid,
X³ represents the natural amino acid P, or an unnatural amino acid selected from a list consisting of 2,3,3a,4,5,6,7,7a-Octahydroindole-2-carboxylic acid (Oic), L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline ((3S—OH)P, L-Pipecolic acid (Pip), (1R,3S,5R)-2-azabicyclo[3.1.0]hexane-3-carboxylic acid, (6S)-5-Azaspiro-[2.4]heptane-6-carboxylic acid, rel-(1R,3R,5R,6R)-6-(trifluoromethyl)-2-azabicyclo[3.1.0]hexane-3-carboxylic acid, (2S)-2-Amino-4,4,4-trifluorobutanoic acid, (2S,3aS,6aS)-octahydrocyclopenta[b]^{¬}pyrrole-2-carboxylic acid, trans-4-fluoroproline ((trans-4-Fluoro)P), (2S,4S)-4-fluoroproline ((cis-4-Fluoro)P), L-4,4-difluoroproline ((Difluoro)P), rel-(3R,6R)-1,1-difluoro-5-azaspiro[2.4]heptane-6-carboxylic acid (enantiomer 1) and rel-(3R,6R)-1,1-difluoro-5-azaspiro[2.4]heptane-6-carboxylic acid (enantiomer 2),
X⁴ represents any natural amino acid or an unnatural amino acid, whereas any natural amino acid and/or unnatural amino acid can be in D- or L-stereoconfiguration,
X⁵ represents a natural amino acid selected from a list consisting of F, H, W or Y or an unnatural amino acid selected from a list consisting of Cyclohexylalanine (Cha), 2,3,3a,4,5,6,7,7a-Octahydrindole-2-carboxylic acid (Oic), L-4-Bromophenylalanine ((4-Bromo)F), 2,5-Difluoro-L-phenylalanine ((2,5-Difluoro)F), 2-Chlor-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bomophenylalanine ((4-Bromo)F), 2-Fluoro-L-phenylalanine ((2-Fluor)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Napthylalanine (1-Nal), L-2-Napthylalanine (2-Nal), (2R)-Amino-(1-methyl-1H-indazol-5-yl)acetic acid and (2S)-3-(indol-4-yl)-2-(amino)propanoic acid,
X⁶ represents any natural amino acid or an unnatural amino acid, whereas any natural amino acid and/or unnatural amino acid can be in D- or L-stereoconfiguration,
wherein any natural amino acid or an unnatural amino acid bearing an amino group might be substituted with 6-Carboxytetramethylrhodamine (Tam) or ##C(O)R³,
wherein
## marks the attachment to the terminal amino group of X¹,
R³ represents C₁-C₆-alkylene, aryl, heteroaryl, C₃-C₈-cycloalkyl or C₃-C₇-heterocycloalkyl,
wherein C₁-C₆-alkylene is up to trisubstituted identically or differently by a radical selected from the group consisting of hydroxyl, methoxy, ethoxy, carboxy, amino and halogen,
wherein aryl, heteroaryl, C₃-C₈-cycloalkyl and C₃-C₇-heterocycloalkyl can be up to trisubstituted identically or differently by a radical selected from the group of C₁-C₄-alkyl, hydroxyl, methoxy, ethoxy, carbonyl, carboxy, amino and halogen,
X⁷ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), (2S)-3-(2,3-difluorophenyl)-2-aminopropanoic acid, L-Phenylglycine (Phg)N-Phenylglycine ((N-Ph)G), 3-Chlorophenylglycine ((3-Chloro-Ph)G), 3-(1,3-Benzothiazol-2-yl)-L-alanine 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal), 3-(Aminomethyl)benzoic acid, L-1-Naphthylalanine (1-Nal), L-2-Naphthylalanine (2-Nal), (2R)-Amino-(1-methyl-1H-indazol-5-yl)acetic acid and (2S)-3-(indol-4-yl)-2-(amino)propanoic acid,
or a pharmaceutically acceptable salt, hydrate, solvate or solvate of the salt thereof,
with the proviso, that compound YFP[cQFAFC] and yFP[xQFAWC], or a pharmaceutically acceptable salt, hydrate, solvate or solvate of the salt of any of the foregoing are excluded.

2: The compound of formula (I) according to claim 1, wherein
R¹ is absent
or
represents 6-Carboxytetramethylrhodamine (Tam), ##C(O)R³ or the sequence R⁴GFLG##,
wherein
## marks the attachment to the terminal amino group of X¹,
R³ represents C₁-C₄-alkylene,
wherein C₁-C₄-alkylene is up to trisubstituted identically or differently by a radical selected from the group consisting of hydroxyl, methoxy, ethoxy, carboxy, amino, fluoro and chloro,
R⁴ represents 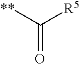
wherein
R⁵ represents C₁-C₄-alkylene,
wherein C₁-C₄-alkylene is up to trisubstituted identically or differently by a radical selected from the group consisting of hydroxyl, methoxy, ethoxy, carboxy, amino, chloro and fluoro,
or 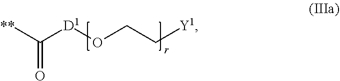
wherein
** marks the attachment to a nitrogen atom,
D¹ is C₁-C₄-alkylene,
Y¹ is selected from the group consisting of hydroxyl, methoxy, ethoxy, carboxy, carboxamide or amino,
wherein amino might be substituted with 6-carboxytetramethylrhodamine (Tam) via an amide bond,
and
r represents an integer of from 2 to 6,
R² represents a group of the formula (II) 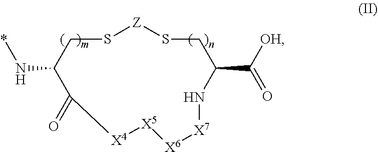
wherein
* represents the attachment to the carbonyl atom of the carboxy group of X³,
Z represents a bond or —CH₂—,
m represents 1 or 2,
n represents 1 or 2,
X¹ represents a natural amino acid selected from a list consisting of L, I, F, H, M, W, Y or y or an unnatural amino acid selected from a list consisting of L-Norleucine (Nle), 2,3,3a,4,5,6,7,7a-Octahydroindole-2-carboxylic acid (Oic), 4-Bromophenylalanine ((4-Bromo)F), 2,5-Difluorophenylalanine ((2,5-Difluoro)F), 2-Chlorophenylalanine ((2-Chloro)F), 3-Chlorophenylalanine ((3-Chloro)F), 4-Chlorophenylalanine ((4-Chloro)F), 2-Bromophenylalanine ((2-Bromo)F), 3-Bromophenylalanine ((3-Bromo)F), 4-Bromophenylalanine ((4-Bromo)F), 2-Fluorophenylalanine ((2-Fluoro)F), 3-Fluorophenylalanine ((3-Fluoro)F), 4-Fluorophenylalanine ((4-Fluoro)F), (2,5-difluoro-phenylalanine, 2-Methyl-phenylalanine ((2-Me)F), 3-Methyl-phenylalanine ((3-Me)F), 4-Methylphenylalanine ((4-Me)F), 1-Benzyl-histidine (H(1-Bn)), 1-Methyl-histidine (H(1-Me)), 3-Methylhistidine (3-Me)H), 2-Pyridylalanine (2-Pal), 3-Pyridylalanine (3-Pal), 4-Pyridylalanine (4-Pal), whereas any natural amino acid and/or unnatural amino acid from that list can be in D- or L-stereoconfiguration,
X² represents a natural amino acid selected from a list consisting of L, I, F, H, M, W or Y or an unnatural amino acid selected from a list consisting of L-Norleucine (Nle), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal),
X³ represents the natural amino acid P, or an unnatural amino acid selected from a list consisting of L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline ((3S—OH)P, (1R,3S,5R)-2-azabicyclo[3.1.0]hexane-3-carboxylic acid, (6S)-5-Aza^{¬}spiro^{¬}[2.4]heptane-6-carboxylic acid, rel-(1R,3R,5R,6R)-6-(trifluoromethyl)-2-azabicyclo[3.1.0]hexane-3-carboxylic acid, (2S)-2-Amino-4,4,4-trifluorobutanoic acid, (2S,3aS,6aS)-octahydrocyclopenta[b]^{¬}pyrrole-2-carboxylic acid, (2S,4S)-4-fluoroproline ((cis-4-Fluoro)P), L-4,4-difluoroproline ((Difluoro)P),
X⁴ represents any natural amino acid, whereas any natural amino acid can be in D- or L-stereoconfiguration,
X⁵ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal),
X⁶ represents any natural amino acid, whereas any natural amino acid can be in D- or L-stereoconfiguration,
wherein the amino group of Lysin might be substituted with 6-Carboxytetramethylrhodamine (Tam) or ##C(O)R³,
wherein
## marks the attachment to the terminal amino group of X¹,
R³ represents C₁-C₆-alkylene, aryl, heteroaryl, C₃-C₈-cycloalkyl or C₃-C₇-heterocycloalkyl,
wherein C₁-C₆-alkylene is up to trisubstituted identically or differently by a radical selected from the group consisting of hydroxyl, methoxy, ethoxy, carboxy, amino and halogen,
wherein aryl, heteroaryl, C₃-C₈-cycloalkyl and C3-C₇-heterocycloalkyl can be up to trisubstituted identically or differently by a radical selected from the group of C₁-C₄-alkyl, hydroxyl, methoxy, ethoxy, carbonyl, carboxy, amino and halogen,
X⁷ represents a natural amino acid selected from a list consisting of F, H, W or Y or an unnatural amino acid selected from a list consisting of Cyclohexylalanine (Cha), 2,3,3a,4,5,6,7,7a-Octahydroindole-2-carboxylic acid (Oic), L-4-Bromophenylalanine ((4-Bromo)F), 2,5-Difluoro-L-phenylalanine ((2,5-Difluoro)F), 2-Chloro-L-phenylalanine ((2-Chloro)F), 3-Chloro-L-phenylalanine ((3-Chloro)F), 4-Chloro-L-phenylalanine ((4-Chloro)F), L-2-Bromophenylalanine ((2-Bromo)F), L-3-Bromophenylalanine ((3-Bromo)F), L-4-Bromophenylalanine ((4-Bromo)F), 2-Fluoro-L-phenylalanine ((2-Fluoro)F), 3-Fluoro-L-phenylalanine ((3-Fluoro)F), 4-Fluoro-L-phenylalanine ((4-Fluoro)F), (2,5-difluoro-L-phenylalanine, 2-Methyl-L-phenylalanine ((2-Me)F), 3-Methyl-L-phenylalanine ((3-Me)F), 4-Methyl-L-phenylalanine ((4-Me)F), 1-Benzyl-L-histidine (H(1-Bn)), 1-Methyl-L-histidine (H(1-Me)), L-3-Methylhistidine (3-Me)H), L-2-Pyridylalanine (2-Pal), L-3-Pyridylalanine (3-Pal), L-4-Pyridylalanine (4-Pal),
or a pharmaceutically acceptable salt, hydrate, solvate or solvate of the salt thereof,
with the proviso, that compounds YFP[cQFAFC] and yFP[xQFAWC], or a pharmaceutically acceptable salt, hydrate, solvate or solvate of the salt of any of the foregoing are excluded.

3: The compound of formula (I) according to claim 1, wherein
R¹ is absent
or
represents 6-Carboxytetramethylrhodamine (Tam) or the sequence R⁴GFLG ##,
wherein
## marks the attachment to the terminal amino group of X¹,
R⁴ represents 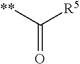
wherein
R⁵ represents methyl or ethyl,
or
represents a group of the formula (IIIa) 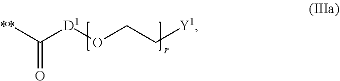
wherein
marks the attachment to a nitrogen atom,
D¹ is C₁-C₄-alkylene,
Y¹ is selected from the group consisting of hydroxyl, methoxy, ethoxy, carboxy, carboxamide or amino,
wherein amino might be substituted with 6-carboxytetramethylrhodamine (Tam) via an amide bond,
and
r represents an integer of from 2 to 4,
R² represents a group of the formula (II) 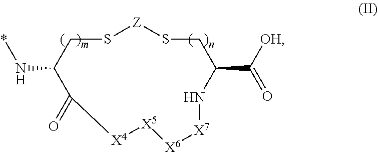
wherein
* represents the attachment to the carbonyl atom of the carboxy group of X³,
Z represents a bond or —CH₂—,
m represents 1 or 2,
n represents 1 or 2,
X¹ represents a natural amino acid selected from a list consisting of F, H, Y or y, whereas any amino acid from that list can be in D- or L-stereoconfiguration,
X² represents a natural amino acid selected from a list consisting of F, H, Y or y, whereas any amino acid from that list can be in D- or L-stereoconfiguration,
X³ represents the natural amino acid P, or an unnatural amino acid selected from a list consisting of L-Hydroxyproline (Hyp), (2S,4S)-4-Trifluoromethyl-pyrrolidine-2-carboxylic acid ((4-CF3)P), (2S,4S)-4-fluoroproline ((cis-4-Fluoro)P), trans-4-fluoroproline ((trans-4-Fluoro)P), (2S)-2-amino-4,4,4-trifluorobutanoic acid, L-trans-3-hydroxyproline, (2S,4S)-4-fluoroproline ((cis-4-Fluoro)P), L-4,4-difluoroproline ((Difluoro)P),
X⁴ represents a natural amino acid selected from a list consisting of Q, A and K, whereas any natural amino acid can be in D- or L-stereoconfiguration,
X⁵ represents a natural amino acid selected from a list consisting of F, H, W or Y,
X⁶ represents a natural amino acid selected from a list consisting of Q, A and K, whereas any natural amino acid can be in D- or L-stereoconfiguration,
wherein the amino group of K might be substituted with 6-Carboxytetramethylrhodamine (Tam),
X⁷ represents a natural amino acid selected from a list consisting of F, H, W or Y,
or a pharmaceutically acceptable salt, hydrate, solvate or solvate of the salt,
with the proviso, that compounds YFP[cQFAFC] and yFP[xQFAWC], or a pharmaceutically acceptable salt, hydrate, solvate or solvate of the salt of any of the foregoing are excluded.

4: The compound of formula (I) according to claim 1, wherein
R¹ is absent
or
represents 6-Carboxytetramethylrhodamine (Tam) or the sequence R⁴GFLG ##,
wherein
## marks the attachment to the terminal amino group of X¹,
R⁴ represents 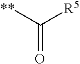
wherein
R⁵ represents methyl,
or
represents a group of the formula (IIIa) 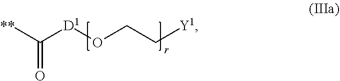
wherein
** marks the attachment to a nitrogen atom,
D¹ is ethylene,
Y¹ is amino,
wherein amino might be substituted with 6-carboxytetramethylrhodamine (Tam) via an amide bond,
and
r represents 4,
R² represents a group of the formula (II) 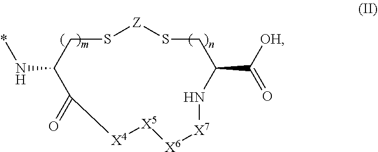
wherein
* represents the attachment to the carbonyl atom of the carboxy group of X³,
Z represents a bond or —CH₂—,
m represents 1 or 2,
n represents 1 or 2,
X¹ represents Y or y,
X² represents F,
X³ represents P,
X⁴ represents Q,
X⁵ represents F,
X⁶ represents A or K,
X⁷ represents For W,
or a pharmaceutically acceptable salt, hydrate, solvate or solvate of the salt thereof,
with the proviso, that compounds YFP[cQFAFC] and yFP[xQFAWC], or a pharmaceutically acceptable salt, hydrate, solvate or solvate of the salt of any of the foregoing are excluded.

5: A method for treatment or prophylaxis of a disease, comprising administering to a human or animal in need thereof an effective amount of a compound according to claim 1, or a pharmaceutically acceptable salt, hydrate, solvate or solvate of the salt thereof.

6: The method of claim 5, wherein the disease is a metabolic disorder, cancer or an inflammatory disorder.

7: The method of claim 5, wherein the disease is diabetes mellitus, obesity, an asthmatic disease, an inflammatory disorder or cancer.

8. (canceled)

9: A pharmaceutical composition comprising a compound according to claim 1, or a pharmaceutically acceptable salt, hydrate, solvate or solvate of the salt thereof, in combination with an inert, non-toxic, pharmaceutically suitable auxiliary.

10. (canceled)

11: A method for treatment or prophylaxis of a disease, comprising administering to a human or animal in need thereof an effective amount of a pharmaceutical composition according to claim 9.

12: The method of claim 11, wherein the disease is diabetes mellitus, obesity, an asthmatic disease, an inflammatory disorder or cancer.

13: The method of claim 11, wherein the disease is a metabolic disorder, cancer or inflammatory disorder.

14: The method of claim 5, wherein the compound is administered to a human.

15: The method of claim 11, wherein the pharmaceutical composition is administered to a human.

16: The compound of formula (I) according to claim 1, wherein the compound is [x4,C9]-chemerin-9 YFP[xQFAFC]:

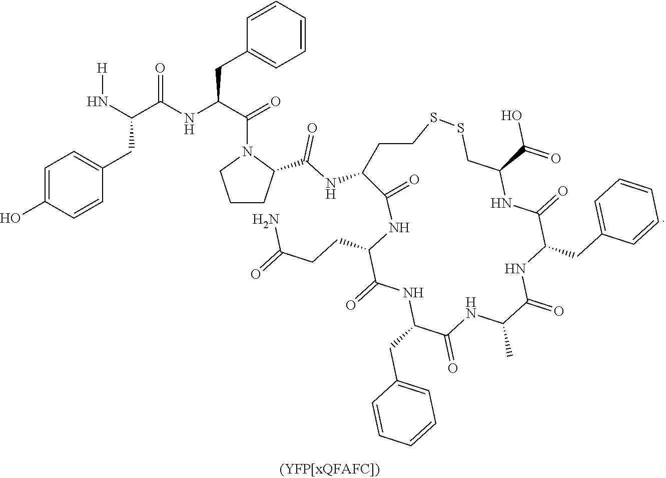 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CCSSC[C@@H](C(=O)O)NC(=O)[C@H](Cc2ccccc2)NC1=O]

17: The compound of formula (I) according to claim 1, wherein the compound is [x4,W8,C9]-chemerin-9 YFP[xQFAWC]:

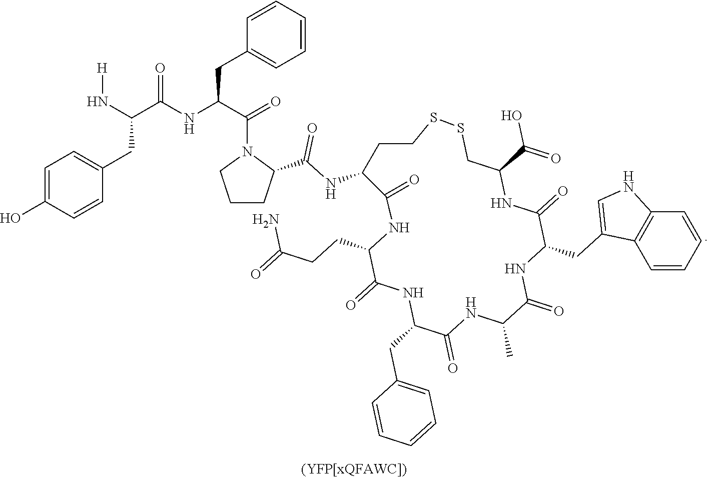 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@@H](N)Cc2ccc(O)cc2)CCSSC[C@@H](C(=O)O)NC(=O)[C@H](Cc2c[nH]c3ccccc23)NC1=O]

18: The compound of formula (I) according to claim 1, wherein the compound is EG4-[x4,C9]-chemerin-9 EG(4)-YFP[xQFAFC]: 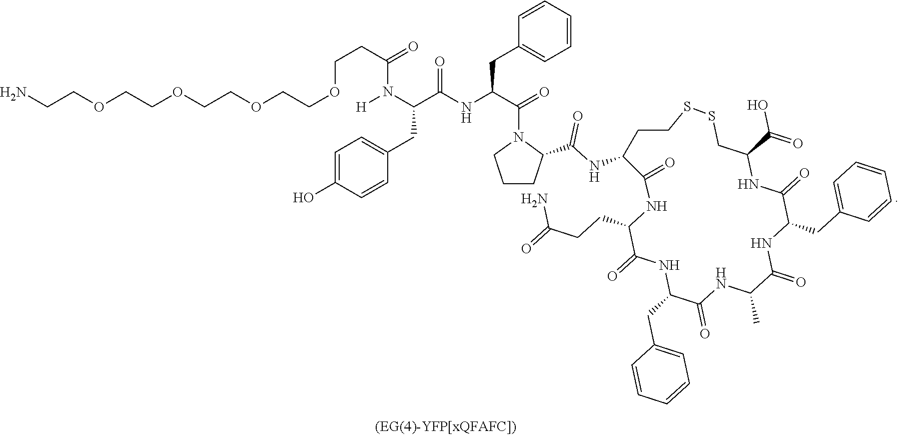 [SMILES: C[C@@H]1NC(=O)[C@H](Cc2ccccc2)NC(=O)[C@H](CCC(N)=O)NC(=O)[C@H](NC(=O)[C@@H]2CCCN2C(=O)[C@H](Cc2ccccc2)NC(=O)[C@H](Cc2ccc(O)cc2)NC(=O)CCOCCOCCOCCOCCN)CCSSC[C@@H](C(=O)O)NC(=O)[C@H](Cc2ccccc2)NC1=O]
